(19) United States
(12) Patent Application Publication
(10) Pub. No.: US 2025/0057951 A1
(43) Pub. Date: Feb. 20, 2025

(54) DISCERNIBLE CELL SURFACE PROTEIN VARIANTS OF CD117 FOR USE IN CELL THERAPY

(71) Applicants: UNIVERSITÄT BASEL, BALE (CH); CIMEIO THERAPEUTICS AG, BASEL (CH)

(72) Inventors: Stefanie URLINGER, MUNCHEN (DE); Rosalba LEPORE, BASEL (CH); Lukas JEKER, RIEHEN (CH); Amélie WIEDERKEHR, RANSPACH LE BAS (FR); Alessandro SINOPOLI, SAINT-LOUIS (FR); Anna CAMUS, BASEL (CH); Lisa WELLINGER, BASEL (CH); Romina MATTER-MARONE, RIEHEN (CH)

(21) Appl. No.: 18/719,780

(22) PCT Filed: Dec. 16, 2022
(86) PCT No.: PCT/EP2022/086452

§ 371 Date: Jun. 14, 2024

(30) Foreign Application Priority Data
Dec. 16, 2021 (EP) 21215028.8
Mar. 28, 2022 (EP) 22164796.9
Nov. 16, 2022 (EP) 22207926.1

(51) Int. Cl. A61K 39/00

(52) U.S. Cl. CPC A61K 39/464429; A61K 39/461; A61K 39/4621; A61K 2239/26

(57) ABSTRACT

The present disclosure relates to the use of cells having discernible surface protein with engineered or naturally occurring mutation(s) but functional surface protein for use in therapy. The present invention also relates to the use of cells having discernible CD117 surface protein variants but functional surface protein for use in therapy, in particular adoptive cell therapy.

The present disclosure relates to the use of cells having discernible surface protein with engineered or naturally occurring mutation(s) but functional surface protein for use in therapy. The present invention also relates to the use of cells having discernible CD117 surface protein variants but functional surface protein for use in therapy, in particular adoptive cell therapy.

### STATEMENT REGARDING FUNDING

The project leading to this application has received funding from the European Research Council (ERC) under the European Union's Horizon 2020 research and innovation programme (grant agreement No 818806).

### BACKGROUND OF THE INVENTION

Cell based immunotherapy is emerging as the third pillar of medicine after small molecule therapy and treatments based on biologics such as recombinant proteins including antibodies. Cellular therapy can be used in oncology for treating hematopoietic malignant diseases, but also other applications such as the treatment of genetic diseases, solid organ tumors and autoimmune diseases are under development. However, cellular therapy can be associated with severe unwanted side effects. Indeed, while cancer immunotherapy with chimeric antigen receptor (CAR) T cells has been successful in targeting and eradicating malignant cells expressing a specific antigen, it does often not discriminate between normal and malignant cells and thus induces destruction of the normal hematopoietic system. Targeted therapies, which include antibody-based therapies, such as conventional monoclonal antibodies, multispecific antibodies, such as T cell engagers (e.g., BiTE's) and cellular therapies, such as CAR cells (e.g. CAR T-cells, CAR NK cells or CAR macrophages), eliminate all cells expressing the target molecule. However, most cancer cell surface antigens are shared with normal hematopoietic or other cells. Thus, to identify targets to kill diseased cells including tumors while avoiding damage to healthy cells is a major challenge for targeted therapies (Perna et al., Cancer Cell (2017) 32:506-519). In particular, in myeloid diseases including myeloid malignancies such as myelodysplastic syndrome (MDS), acute myeloid leukemia (AML) or Blastic Plasmacytoid Dendritic Cell Neoplasm (BPDCN) cell surface antigens such as CD117, CD33, or CD123 are shared with normal myeloid progenitors. Therefore, immunotherapy targeting CD117, CD33 or CD123 antigen for MDS, AML or PBDCN can be associated with depletion of normal hematopoietic cells in addition to malignant cells in patients (Gill S. I. Best practice & Research Clinical Hematology, 2019). As a consequence, targeted immunotherapy including mAbs, T cell engagers or CAR T have mostly been elusive, in part owing to the absence of truly disease-specific surface antigens (Gill S. I. Best practice & Research Clinical Hematology, 2019).

To regenerate normal hematopoiesis depleted through CD33-CAR T cell transfer, CD33 CAR T cell resistant hematopoietic cells are being engineered in such a way that the entire CD33 gene is knocked out (Kim et al. 2018. Cell. 173:1439-53). However, CD33 has a constitutive inhibitory effect on myeloid cells through its immunoreceptor tyrosine-based inhibitory motif (ITIM) signaling domain. Thus, it remains unclear how well the loss of CD33 may be tolerated (Wißfeld et al. Glia (2021) 69:1393-1412). CD33-knock-out (CD33 KO) engineered cells transplanted in patients could present long-term functional defects (WO2018/160768, Kim et al. 2018. Cell. 173:1439-53, Borot et al. 2019. PNAS. 116:11978-87, Humbert et al. 2019. Leukemia. 33:762-808). In fact, the frequency of CD33 KO cells decreased in the two monkeys for which a long-term observation was reported. This could indicate functional impairment of CD33 KO cells, for instance through reduced engraftment of CD33 KO long-term repopulating HSC (LT-HSC) or through a competitive disadvantage (Kim et al. 2018. Cell. 173:1439-53). In addition, the number of cell surface antigens with dispensable function is very limited and loss of said redundant cell surface antigen can induce antigen negative relapse. CD19-negative relapses are observed in approximately 30% of patients receiving CD19-targeted CAR T therapy (Orlando et al. 2018 Nat Med 24:1504-6). Dual targeting of CD19 and CD123 can prevent antigen-loss relapses (Ruella et al. 2016 J Clin Invest 126:3814-26).

The inventors in previous patent applications showed that a single amino acid difference in surface protein variants can be engineered into hematopoietic cells to change the antigenicity and be discriminated by specific and selective antibodies (WO2017/186718, WO2018/083071). Contrary to CD33 KO cells, the surface protein variants in these cells retain their normal expression and function and enable to target surface proteins with important non-redundant functions.

CD117 (also referred to as c-kit or Stem Cell Factor Receptor (SCRF)) is a single transmembrane, receptor tyrosine kinase that binds the ligand Stem Cell Factor (SCF). SCF induces homodimerization of CD117 which activates its tyrosine kinase activity and signals through both the PI3K-AKT and MAPK pathways (Kindblom et al., Am J. Path. 1998 152 (5): 1 259).

CD117 was initially discovered as an oncogene and has been studied in the field of oncology (see, for example, Stankov et al. (2014) Curr Pharm Des. 20:2849-80). CD117 is highly expressed on hematopoietic stem and progenitor cells (HSCs). This expression pattern makes CD117 a potential target for conditioning across a broad range of diseases (Russkamp et al. Exp. Hematol. (2021) 95:31-45; Czechowicz et al. Nat Commun (2019) 10:617). There remains, however, a need for anti-CD117 based therapy that is effective for conditioning a patient for transplantation, such as a bone marrow transplantation.

The present disclosure aimed to identify amino acid residues of CD117 that are exposed on the cell surface and that can be substituted in a manner such that a) the function of CD117 is not, or at least not substantially, altered, i.e. the variant of CD117 is functionally indistinguishable from the wild type version of CD117, and b) a moiety, such as an antibody or a CAR T cell, that binds to the wild type version of CD117, but shows a substantially decreased or no binding to the altered version of CD117, i.e. the variant of CD117 is immunologically distinguishable from the wild type version of CD117. Most single amino acid substitution in any given target protein will only affect the binding of a moiety, if the amino acid substitution is part of, or is close to, the epitope of the binding moiety. As also will be appreciated, many single amino acid substitutions that do affect binding of a binding moiety to a target antigen will also impact the functionality of the target antigen. It is therefore a highly sophisticated and unpredictable task to identify those amino acid substitutions that fulfill both requirements that do affect binding a moiety to a target antigen and which at the same time do not, or not substantially, affect its function.

Several anti-CD117 moieties are known in the art, some of which are currently in development. Antibody SR-1 was originally isolated from a hybridoma (WO1992017505). Humanized versions of SR-1 were generated (WO2007127317; WO2020112687). Anti-CD117 drug conjugates are described in WO2016020791. Other anti-CD117 antibodies are described in WO2015050959 and WO2019084064. Certain anti-CD117 antibodies are also available commercially, e.g. antibody 104D2 Dianova (#117PE-100T). These and other anti-CD117 moieties may be used in the context of the present disclosure. WO2021041945 discloses genomic alterations of antigens, including CD117, generated by base editing. These antigen alterations were however not characterized, in particular the biological function of the variants was not tested.

### SUMMARY OF THE INVENTION

One of the objectives of the present disclosure is to develop a safer method for the treatment of malignancies, in particular cancer, hematological malignancies, myeloid diseases. The inventors thus sought variations of the surface protein CD117 which are immunologically distinguishable while retaining or substantially retaining normal function, and where amino acid changes originate from a single or multiple amino acid or nucleotide variations. In particular, the inventors identified rationally designed and naturally occurring variants of CD117 and showed that these mutations change the antigenicity of CD117 to a specific antibody while retaining its normal expression and function, in particular binding to SCF, SCF-dependent proliferation and/or SCF-dependent phosphorylation.

The present disclosure relates to a mammalian cell or a population of cells expressing a first isoform of CD117 for use in a medical treatment in a patient in need thereof, said patient having cells expressing a second isoform of CD117, wherein said cell expressing said first isoform comprises genomic DNA with at least one polymorphism or genetically engineered allele, wherein said polymorphism or genetically engineered allele is not present in the genome of the patient having cells expressing said second isoform of CD117 and preferably wherein said first and second isoforms are functional. Alternatively said first isoform is generated via RNA editing.

In a particular embodiment, the present disclosure relates to the mammalian cell or population of cells, preferably hematopoietic stem cells for use in a medical treatment in a patient in need thereof wherein said medical treatment comprises: administering a therapeutically efficient amount of said cell or population of cells expressing said first isoform of CD117 to said patient in need thereof, in combination with a therapeutically efficient amount of a depleting agent comprising at least a first antigen-binding region that binds specifically to said second isoform of CD117 to specifically deplete patient cells expressing said second isoform of CD117, preferably to restore normal hematopoiesis after immunotherapy in the treatment of hematopoietic disease, preferably malignant hematopoietic disease such as acute myeloid leukemia (AML), myeloblastic syndrome (MDS), blastic plasmacytoid dendritic cell neoplasm (BPDCN), chronic myeloid leukemia (CML), B-acute lymphoblastic leukemia (B-ALL) or mastocytosis.

In other embodiments the medical treatment relates to the restoration of the hematopoietic or the immune function in genetic diseases of the hematopoietic or immune system, such as severe combined immunodeficiency syndrome (SCID), sickle cell disease (SCD), beta-thalassemia, Fanconi anemia or Diamond-Blackfan anemia.

In other embodiments the medical treatment relates to the restoration of the normal function in genetic diseases that are not originating in the hematopoietic and immune system but that can be treated by use of modified hematopoietic cells.

In other embodiments the medical treatment relates to the restoration of the normal immune function in autoimmune diseases, such as systemic lupus erythematosus (SLE), systemic sclerosis (SSc) or multiple sclerosis (MS).

In another particular embodiment, the present disclosure relates to the mammalian cell or population of cells for use in a medical treatment in a patient in need thereof, wherein said medical treatment comprises: administering a therapeutically efficient amount of said cell or population of cells expressing said first isoform to said patient in need thereof in combination with a therapeutically efficient amount of a depleting agent comprising at least a second antigen-binding region that binds specifically to said first isoform to specifically deplete transferred cells expressing first isoform, preferably for use in adoptive cell transfer therapy, more preferably for the treatment of malignant hematopoietic disease such as acute myeloid leukemia (AML), myeloblastic syndrome (MDS), blastic plasmacytoid dendritic cell neoplasm (BPDCN), chronic myeloid leukemia (CML), B-acute lymphoblastic leukemia (B-ALL) or mastocytosis, again more preferably wherein said depleting agent is administered subsequently to said cell or population of cells expressing said first isoform of surface protein to avoid eventual severe side effects such as graft-versus-host disease due to the transplantation.

In another aspect, the present disclosure relates to a pharmaceutical composition comprising a mammalian cell, preferably a hematopoietic stem cell or an immune cell such as T-cell as described above and preferably a depleting agent and a pharmaceutically acceptable carrier.

The present disclosure also relates to a depleting agent for use in preventing or reducing the risk of severe side effects in a patient having received a cell expressing a first isoform of CD117, wherein said patient's native cells express a second isoform of CD117, and wherein said depleting agent comprises at least a second antigen-binding region which binds specifically to said first isoform of CD117 and does not bind to said second isoform of CD117. In certain embodiments said depleting agent binds substantially weaker to said second isoform of CD117.

The present disclosure also relates to a depleting agent for use in preventing or reducing the risk of severe side effects in a patient having received a cell expressing a first isoform of CD117, wherein said patient's native cells express a second isoform of CD117, and wherein said depleting agent comprises at least a second antigen-binding region which binds specifically to said first isoform of CD117 and does not bind to said second isoform of CD117, and wherein said first and second isoforms are substantially functionally identical.

In certain embodiments said depleting agent binds substantially weaker to said second isoform of CD117.

The present disclosure also relates to a depleting agent for use in preventing or reducing the risk of severe side effects in a patient having received a cell expressing a first isoform of CD117, wherein said patient's native cells express a second isoform of CD117, and wherein said depleting agent comprises at least a second antigen-binding region which binds specifically to said first isoform of CD117 and does not bind to said second isoform of CD117, and wherein said first and second isoforms bind to SCF, lead to SCF-dependent proliferation and/or lead to SCF-dependent phosphorylation. In certain embodiments said depleting agent binds substantially weaker to said second isoform of CD117.

The present disclosure also relates to a depleting agent for use in preventing or reducing the risk of severe side effects in a patient having received a cell expressing a first isoform of CD117, wherein said patient's native cells express a second isoform of CD117, and wherein said depleting agent comprises at least a second antigen-binding region which binds specifically to said first isoform of CD117 and does not bind to said second isoform of CD117, wherein said polymorphic or genetically engineered allele is characterized by at least one substitution of an amino acid in position E73, T74, V120, D121, R122, S123, Y125, K127, K193, I201, K203, S239, Y259, N260, S261, D266, Y269 or R271 of SEQ ID NO: 1, preferably by at least one, substitution of an amino acid in position E73, V120, D121, R122, S123, K127, K193, S239, Y259 or S261 of SEQ ID NO: 1, more preferably by at least one substitution of an amino acid in position E73, D121, R122, S123, S239, Y259 or S261, and most preferably by at least one substitution of an amino acid in position E73, D121, or S123 of SEQ ID NO: 1. In certain embodiments said depleting agent binds substantially weaker to said second isoform of CD117.

The present disclosure also relates to a depleting agent for use in preventing or reducing the risk of severe side effects in a patient having received a cell expressing a first isoform of CD117, wherein said patient's native cells express a second isoform of CD117, and wherein said depleting agent comprises at least a second antigen-binding region which binds specifically to said first isoform of CD117 and does not bind to said second isoform of CD117, wherein amino acid residue E73 is substituted with an amino acid selected from the group consisting of K, L, Q, G, A, Y, R, F, I, M, P and W or where residue E73 is deleted, preferably selected from the group consisting K, L, Q, G, A, Y and R, more preferably selected from the group consisting of K, Y, and R, and/or residue D121 is substituted with S, V, Y, H, K, R or T, more preferably Y, H, K, R or T, and most preferably H or K, and/or residue S123 is substituted with V, I, L, P, F, Y, M, D, E, K or R, more preferably with P, F or K, and most preferably K, and/or residue S239 is substituted with an amino acid selected from the group consisting of H and K, and/or residue Y259 is substituted with an amino acid selected from the group consisting of E, A, G, P, C and H, preferably P, A or G, most preferably A, and/or residue K193 is substituted with an amino acid selected from the group consisting G, T, M, D and E. In certain embodiments said depleting agent binds substantially weaker to said second isoform of CD117.

The present disclosure also relates to a method for improving engraftment of hematopoietic stem cell transplants. Conditioning (depletion of HSCs) prior to hematopoietic stem cell transplantation (HSCT) is used to promote engraftment. Indeed, conditioning efficacy is associated with improved engraftment. One disadvantage of myeloablative (toxic) conditioning is that it can lead to increasing levels of chimerism. Avoiding toxic conditioning is an important goal that can be achieved with the present disclosure. Current methods for conditioning involve the use of intravenous busulfan. Busulfan is a DNA alkylating drug originally designed to treat hematologic diseases, such as acute myeloid leukemia (AML). However, busulfan carries the risk of significant side effects, including sterility, primary or secondary malignancy, and additional acute and chronic toxicities.

The present disclosure also relates to an anti-CD117 agent which competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22. In certain embodiments said antigen binding region comprises the following mutations:
a) the asparagine in the VLCDR1 region (SEQ ID NO: 20) is replaced by glutamic acid, and
b) the aspartic acid in the VLCDR3 region (SEQ ID NO: 22) is replaced by glutamic acid, and optionally,
c) the second asparagine in the VLCDR3 region (SEQ ID NO: 22) is replaced by lysine. Preferably said-anti-CD117 agent does not bind to a E73K variant of CD117. Also preferably said-anti-CD117 agent does not bind to a D121K variant of CD117. Also preferably said-anti-CD117 agent does not bind to a S123K variant of CD117.

The present disclosure also relates to an anti-CD117 agent which competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 25, VHCDR2 is SEQ ID NO: 26 and VHCDR3 is SEQ ID NO: 27; and
b) an antibody light chain variable domain (VL) comprising the three CDRs VLCDR1, VLCDR2 and VLCDR3 wherein VLCDR1 is SEQ ID NO: 28, VLCDR2 is SEQ ID NO: 29 and VLCDR3 is SEQ ID NO: 30.

The present disclosure also relates to an anti-CD117 agent which competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 33, VHCDR2 is SEQ ID NO: 34 and VHCDR3 is SEQ ID NO: 35; and
b) an antibody light chain variable domain (VL) comprising the three CDRs VLCDR1, VLCDR2 and VLCDR3 wherein VLCDR1 is SEQ ID NO: 36, VLCDR2 is SEQ ID NO: 37 and VLCDR3 is SEQ ID NO: 38.

### FIGURE LEGENDS

FIG. 1 shows the binding of anti-CD117 antibodies to HEK-293T cells transfected with human wild-type CD117 or with an empty vector. Serial dilutions of each antibody were tested for immunoreactivity via flow cytometry. All six antibodies in Mab format bind to human CD117 in a concentration dependent manner. CD117-negative HEK-293T cells (transfected with an empty vector) did not show any antibody binding.

FIG. 2 shows the binding of anti-CD117 Fab fragments to HEK-293T cells transfected with human wild-type CD117 or with an empty vector. Like the full length antibodies, also the Fab fragments bind to human CD117 in a concentration dependent manner. CD117-negative HEK-293T cells (transfected with an empty vector) did not show any antibody binding.

FIG. 3 shows the result of an alanine scan on human CD117 extracellular domain for each of the six antibodies tested. For each mutant CD117 clone, the mean binding value determined by flow cytometry was plotted as a function of expression. Clones that were identified as critical for antibody binding are shown as grey circles. Shown in open circles are secondary clones, i.e. clones that did not meet the initially set thresholds but whose decreased binding activity and proximity to critical residues suggested that the mutated residue may be part of the antibody epitope.

FIG. 4 shows the aggregation behaviour of the elected, recombinant purified CD117 variants. The line indicates the monomeric content of wild type CD117 recombinant purified protein. The monomeric content was estimated from the preparative size-exclusion chromatograms. The monomeric content of most variants was acceptable. ND stands for non-determinable.

FIG. 5 shows the production yield obtained for the elected recombinant purified CD117 variants. The line indicates the yield of wild type CD117. The production yield for all but a very few variants was sufficiently high.

FIG. 6 shows the t½ melting temperatures measured for the elected recombinant purified CD117 variants. The line indicates the melting temperature of wild type CD117. For one variant, V120P, two melting points were measured.

FIG. 7 shows the binding of antibody Refmab #1 to elected recombinant purified variants of human CD117 by comparing the nm shift at the end of association to the WT.

FIG. 8 shows the binding affinity of antibody Refmab #1 to elected recombinant purified variants of human CD117. Panel A: KD of all variants against Refmab #1 was determined. Solid circles illustrate the measured KDs determined from two individual measurements, while the empty circles illustrate binding affinities higher than 2 μM with no observed nm shifts or shifts below 0.1 nm. Error bars indicate the standard deviation. Panel B: the nm shift at the end of the association (600 s) is plotted against the antigen concentration used, ranging from 2000-5 nM. Variants D121K, S123K and S123E do not show any binding up to 2000 nM. The error bars indicate the standard deviation of two individual measurements.

FIG. 9 shows the binding of elected recombinant purified variants of human CD117 to SCF. Increase in the KD of binding of SCF to wild-type human CD117 (measured with construct CD117 D1-2-3, carrying domains 1, 2, and 3 which harbor the binding site for SCF) is shown. “Not determinable” means that binding was observed but no KD could be determined, while “no binding” stands for no observed binding.

FIG. 10 shows the binding of recombinant purified elected variants of human CD117 to SCF. Increase in the KD of binding of SCF to wild-type human CD117 (measured with construct CD117 D1-2-3, carrying domains 1, 2, and 3 which harbor the binding site for SCF) is shown. The binding was analyzed by using seven concentrations of CD117 variants. The error bars indicate the standard deviation from two individual measurements. KD increase below 1 corresponds to stronger SCF binding of the variant compared to CD117 WT and a KD increase above 1 corresponds to a weaker SCF binding of the variant compared to CD117 WT.

FIG. 11 demonstrates that wild type TF-1 cells, as well as cells carrying a E73K knock-in of CD117, proliferate upon stimulation with 100 ng/ml SCF. With increasing concentrations of Refmab #1 proliferation decreases for wild type cells, but not for E73K cells. Knock-in cells designated as “KI 1” and “KI 2” were obtained from two independent knock-in experiments and subsequently FACS sorted to obtain pure E73K knock-in populations.

FIG. 12 demonstrates that SCF-induced phosphorylation can be blocked with an antibody (Refmab #1) in wild type CD117 TF-1 cells, whereas SCF-induced phosphorylation is unaffected with the same antibody in the E73K variant of CD117 TF-1 cells. CD117 knock-out (ko) TF-1 cells do not show SCF-induced CD117 phosphorylation.

FIG. 13 shows a map depicting binding of the tested crRNAs relative to position E73.

FIG. 14 shows a map depicting binding of the tested crRNAs relative to positions 120-123.

FIG. 15 shows a qualitative analysis of the binding of antibodies Refmab #2 and Refmab #3 to elected recombinant purified variants of human CD117. “Not determinable” means that binding was observed but no KD could be quantified due to biphasic behaviour.

FIG. 16 shows that RefMab #1 had a pronounced inhibitory effect on the proliferation of wild type TF-1 cells, whereas proliferation of TF-1 cells with knock-ins of all CD117 variants tested was not affected by RefMab #1.

FIG. 17 shows that binding of Refmab #1 is completely abolished in TF-1 cells expressing CD117 variants E73K, E73Y, D121K and S123K, while wildtype TF-1 cells expressing wt CD117 are bound.

FIG. 18 shows the SCF-dependent phosphorylation of CD117 at position Tyr719 of wild-type TF-1 cells and two independently generated knock-in populations of E73K, a variant of CD117. D121K and S123K variants of CD117 behaved similarly (data not shown).

FIG. 19 shows that SCF-dependent CD117 phosphorylation in wild-type TF-1 cells is blocked by antibodies Refmab #1-Ernie (referred to as “Ernie”) and Refmab #1-Bert (referred to as “Bert”), whereas in TF-1 cells with D121K and S123K variants of CD117 phosphorylation is unaffected. The same is also observed for variants E73K, E73Y and S123F (data not shown).

FIG. 20 shows that antibody Refmab #1-Bert effectively depletes unedited, wildtype human HSPCs from CD34+ humanized mice in vivo.

FIG. 21 shows that whereas the isotype control antibody depletes neither unedited wildtype CD117+ myeloid cells nor E73K edited cells in vivo, antibody Refmab #1-Bert depletes unedited wildtype CD117+ myeloid while it spares E73K variant CD117+ myeloid cells. In the presence of the isotype control antibody, CD34+ human E73K variant CD117 cells engraft essentially as well as unedited cells CD34+ human cells in immune-deficient mice.

FIG. 22 shows that wild-type DF-1 cells, but not DF-1 cells transfected with D121K or S123K variants of CD117, are depleted by the tested ADCs (Refmab #1-Ernie-teserine (A) and Refmab #1-Bert-teserine (B)). D121K A5 and D121K A6 designate two different batches of plasmids for the same variant.

FIG. 23 shows the depletion of unedited CD34+ progenitor cells (gated as live/hCD45+/CD34+/CD38−) in mice receiving antibody Refmab #1-Bert compared to mice receiving an isotype control antibody. In contrast, injection of Refmab #1-Bert resulted in an enrichment of E73K, D121K and S123K-edited variant cells compared to animals receiving isotype control antibody. Unedited and edited cells were identified by staining with the CD117-specific antibody clones 104D2 and SR-1. Cells were classified as unedited (104D2+ and SR-1+) or edited (104D2+ but SR-1−).

### DETAILED DESCRIPTION OF THE INVENTION

Immunotherapy is a promising therapy to treat cancer, genetic and autoimmune diseases. Immunodepleting agent such as antibodies or engineered immune cells directed to tumor antigen are administered into a patient to target and kill tumor cells. However, as tumor surface proteins are also expressed at the surface of normal cells including hematopoietic cells, this strategy can induce severe side effects to the patients, e.g. by altering hematopoiesis. To restore hematopoiesis in the patient, hematopoietic cells can be subsequently transplanted into the patient. However, the binding of the depleting agent not only to the diseased cells but also to the newly transplanted healthy cells can limit the maximal tolerated dose or limit the use to treatment before transplantation of healthy cells. Alternatively, transplanted cells need to be resistant to said immunodepleting agent in order not to be targeted and eliminated by it. One approach is therefore to select cells resistant to said immunodepleting agent used in immunotherapy while retaining their function to restore normal hematopoiesis in the patient.

The inventors develop a method to identify functional allelic variants in the genetic sequence encoding the surface protein region responsible for the binding of a specific depleting agent. Such variants can be naturally occurring polymorphisms and/or designed and engineered variants. Different isoforms of surface proteins can be selected or generated. Said first isoform of a surface protein encoded by a nucleic acid with said polymorphism is not recognized by a specific depleting agent. This variant allele particularly does not alter or does not substantially alter the function of the surface protein. Thus, said depleting agent can be used to bind specifically to the one isoform and not, or not substantially, the other isoform thereby depleting specifically cells expressing one isoform. For example, if the depleting agent binds specifically to the second isoform, but not the first isoform, said depleting agent will specifically deplete cells expressing said second isoform. In another embodiment, said first isoform can be recognized by a second agent and thus this second agent can be used to deplete specifically cells expressing the first isoform, but not second isoform. The cells expressing the first isoform of the surface protein encoded by at least one variant allele is advantageously used in medical treatment in a patient having cells expressing a second isoform, in particular for depleting specifically transplanted or patient cells by using a second or first agent respectively.

Without a sophisticated in silico analysis, it is impossible to predict which mutation in a surface antigen can be used in such an approach. First, the mutations need to lie on a surface exposed stretch of the surface antigen that is accessible for the depleting agent. Second, the depleting agent needs to bind to this stretch on the exposed area of the surface antigen. Third, binding needs to be affected sufficiently enough so that the depleting agent can discriminate the first isoform from the second isoform. Residual binding to the other isoform should be minimal or, better, be completely absent. Fourth, the mutation should not affect, or only marginally affect, the function of the surface antigen. The mutated isoform should fulfill its biological function at least to an extent that is tolerable in a given therapeutic setting. Although in silico methods can assist in identifying and prioritizing optimal candidates, experimental testing is necessary to validate the usefulness of any given mutation.

### Depleting Agent

The present disclosure relates to an agent comprising an antigen binding region which binds specifically to one isoform of CD117 on a cell and does not bind or binds substantially weaker to another isoform of CD117. Such agent is referred to herein as “depleting agent”. Both isoforms of CD117 are functional, i.e. CD117 is functional with respect to at least one relevant property. Preferably both isoforms of CD117 have that same function, i.e., they are functionally indistinguishable.

The two isoforms of CD117 differ however with respect to binding to the depleting agent. The depleting agent only binds specifically to one of the isoforms of CD117. The isoforms can therefore be described as functional identical (or functionally substantially identical), but immunologically distinguishable.

The first isoform and the second isoform of CD117 may be polymorphic alleles. Preferably, the first isoform and the second isoform of CD117 are naturally occurring polymorphic alleles. Also preferably, the first isoform and the second isoform of CD117 are single nucleotide polymorphism (SNP) alleles.

The first isoform and the second isoform of CD117 may also be genetically engineered alleles. Preferably the first isoform and the second isoform of CD117 differ by one, two, three, four or five amino acids. Most preferably the first isoform and the second isoform of CD117 differ by one amino acid.

Various methods can be used to determine the mutation that is to be introduced into CD117 to generate the second isoform. For example, mutations can be randomly inserted, followed by the functional and immunological screening of the variants generated. Alternatively, mutations can be rationally designed, for example by analysis of the secondary or tertiary protein structure of CD117.

The depleting agent comprises an antigen binding region which binds specifically to one isoform of CD117 on a cell and does not bind or binds substantially weaker to another isoform. The depleting agent of the present disclosure can be divided into two main categories.

First, the depleting agent can be a polypeptide comprising an antigen binding region. Said polypeptide may consist of one or more polypeptide chains. Preferably said polypeptide comprising an antigen binding region is an antibody. Said polypeptide comprising an antigen binding region may also be an antibody fragment, an antibody drug conjugate, or another variant of an antibody or scaffold. Exemplary antibody fragments and scaffolds include single domain antibodies, maxibodies, minibodies, intrabodies, diabodies, triabodies, tetrabodies, v-NAR, bis-scFv, camelid antibodies, ankyrins, centyrins, domain antibodies, lipocalins, small modular immuno-pharmaceuticals, maxybodies, Protein A and affilins.

Said polypeptide comprising an antigen binding region may also be a bispecific, biparatopic or multispecific antibody. Such molecules may also contain additional functional domains. For example, said polypeptide comprising an antigen binding region may be a T cell engager, for example a BiTE. Said polypeptide comprising an antigen binding region may also be fused to a cytokine or a chemokine, a toxin or to the extracellular domain of a cell surface receptor.

Alternative, the depleting agent can be a cell comprising an antigen binding region. For example, the depleting agent can be a chimeric antigen receptor (CAR). In certain embodiments of the present disclosure said cell comprising an antigen binding region is a CAR T-cell, CAR NK cells or CAR macrophages. In a preferred embodiment of the present disclosure said cell comprising an antigen binding region is a CAR T-cell. In another preferred embodiment of the present disclosure said cell comprising an antigen binding region is a primary T cell comprising a CAR.

The depleting agent binds specifically to one isoform of CD117, but not the second isoform and thus specifically depletes cells expressing one isoform.

In certain embodiments, the present disclosure relates to an agent comprising a first antigen binding region which binds specifically to a second isoform of CD117 and does not bind a first isoform. In other embodiments, the present disclosure also relates to an agent comprising a second antigen binding region which binds specifically to the first isoform of CD117 and does not bind a second isoform. In certain embodiments said agents binds substantially weaker to said second isoform of CD117.

The first and the second isoform of CD117 may differ from each other by only one amino acid substitution. Said one amino acid difference between the first and the second isoform in may also be the result of the presence of a single nucleotide polymorphism, such as a naturally occurring single nucleotide polymorphism. The first and the second isoform of CD117 may also differ from each other by more than one amino acid, such as by two, by three or by more than three amino acids. The first and the second isoform of CD117 may also differ from each other in that one of the isoforms has an insertion of one, of two, of three or of more than three amino acids compared to the other isoform. The first and the second isoform of CD117 may also differ from each other in that one of the isoforms has a deletion of one, of two, of three or of more than three amino acids compared to the other isoform. The two isoforms may also differ from each other by combinations of amino acid substitutions, insertions and/or deletions. In a preferred embodiment, said depleting agent is an antibody or an antigen-binding fragment. If the two isoforms of CD117 differ by more than one amino acid, then the amino acids changed may be adjacent to each other, i.e., direct neighboring amino acids, or they may be separated.

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain variable region. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In specific embodiments, an antibody provided herein is an antibody fragment, and more particularly any protein including an antigen-binding domain of an antibody as disclosed herein. The antigen-binding domain may also be integrated into another protein scaffold Antibody fragments and scaffolds include, but are not limited to, Fv, Fab, F(ab′) 2, Fab′, dsFv, scFv, sc (Fv) 2, diabodies, single domain antibodies, maxibodies, minibodies, intrabodies, diabodies, triabodies, tetrabodies, v-NAR, bis-scFv, camelid antibodies, ankyrins, centyrins, domain antibodies, lipocalins, small modular immuno-pharmaceuticals, maxybodies, Protein A and affilins.

As used herein, an “antigen binding region” or “antigen-binding fragment of an antibody” means a part of an antibody, i.e. a molecule corresponding to a portion of the structure of the antibody, that exhibits antigen-binding capacity for a specific antigen, possibly in its native form; such fragment especially exhibits the same or substantially the same antigen-binding specificity for said antigen compared to the antigen-binding specificity of the corresponding four-chain antibody. The antigen-binding capacity can be determined by measuring the affinity between the antibody and the target fragment. This antigen-binding region may also be designated as “functional fragments” of antibodies.

The agents of the disclosure comprise antibodies and fragments thereof but also comprise artificial proteins with the capacity to bind antigens mimicking that of antibodies, also termed herein antigen-binding antibody mimetic. Antigen-binding antibody mimetics are organic compounds that specifically bind antigens, but that are not structurally related to antibodies. They are usually artificial peptides or small proteins with a molar mass of about 3 to 20 kDa.

The phrases “an antigen binding region recognizing an antigen” and “an antigen binding region having specificity for an antigen” are used interchangeably herein with the term “an antigen binding region which binds specifically to an antigen”. As used herein, the term “specificity” refers to the ability of an agent comprising an antigen binding region such as an antibody to detectably bind an epitope presented on an antigen.

“Specific binding” or “specifically bind to” includes binding with a monovalent affinity of about 10⁻⁸ M (KD) or stronger. Preferably, binding is considered specific when the binding affinity is between 10⁻⁸ M (KD) and 10⁻¹² M (KD), optionally between 10⁻⁸ M (KD) and 10⁻¹⁰ M (KD), in particular at least 10⁻⁸ M (KD). The affinity can be determined by various methods well known from the one skilled in the art. These methods include, but are not limited to, surface plasmon resonance (SPR), biolayer interferometry (BLI), microscale thermophoresis (MST) and Scatchard plot. Whether a binding domain specifically reacts with or binds to a target can be tested readily by, inter alia, comparing the reaction of said binding domain with a target protein or antigen with the reaction of said binding domain with proteins or antigens other than the target protein.

As used herein, the term “epitope” means the part of an antigen to which the antibody or antigen binding region thereof binds. The epitopes of protein antigens can be divided into two categories, conformational epitope and linear epitope. A conformational epitope corresponds to discontinuous sections of the antigen's amino acid sequence. A linear epitope corresponds to a continuous sequence of amino acids from the antigen.

In another aspect, it is further disclosed herein bispecific or multispecific molecules, such as bispecific antibodies or multispecific antibodies. For example, an antibody can be derivatized or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multi-specific molecules that bind to more than two different binding sites and/or target molecules; such multi-specific molecules are also intended to be encompassed by the terms “bispecific molecule”, “bispecific antibody”, “biparatopic molecule”, “biparatopic antibody”, “multispecific molecule” and “multispecific antibody” as used herein. To create a bispecific molecule, an antibody of the disclosure can be functionally linked (e.g., by chemical coupling, genetic fusion, disulfide bonds, noncovalent association or otherwise) to one or more other binding molecules, such as another antibody, antibody fragment, peptide or binding mimetic, cytokine, chemokine, toxin or a receptor extracellular domain, such that a bispecific molecule results. Specific bispecific and multispecific molecules contemplated by the present disclosure are T cell engagers, such as bispecific T cell engager, for example a BiTE.

As used herein, an agent which does not bind or binds substantially weaker to a particular isoform of CD117 includes an agent which is not able to bind to cells expressing said particular isoform. For experimental testing said agent may be labelled with a fluorescent marker or may be detected with a secondary antibody directed against said agent, and the percentage of cells presenting said fluorescent marker or said secondary antibody is determined by FACS analysis. Typically testing is done in cell lines expressing the recombinant target protein, i.e. CD117. The target protein may be expressed in its entirety. Alternatively, a truncated version may be used, wherein said truncated version at a minimum needs to include the extra cellular domain or the regions of the extracellular domain containing the respective antibody epitope. In order to monitor the expression of the variant isoforms, cells may be stained with two agents simultaneously, one binding the epitope where variants were introduced and a second one that binds an epitope that is different from the one bound by the first agent. The second epitope remains unaltered and thus this staining serves as an expression control. As a non-binding control, cells are used that do not express the protein of interest. As a maximum binding control, cells that normally do not express the protein of interest are transfected with the wildtype isoform. Different cell lines have different expression levels, but the expression is controlled through endogenous control elements such as promoters. Such cell lines can also be used to study the mode-of-action of a depleting agent, the effective shielding against a different mode-of-action, to test cytotoxicity and shielding/resistance from cytotoxicity or to test the function of the engineered receptors. Western Blot, ELISA or FACS can be used to analyze phosphorylation of signaling molecules. Analysis of gene expression changes can serve to analyse gene expression compared to normal function. FACS-based assays can be used to analyse the binding of SCF to CD117 on cells. Cells can also be used to demonstrate the feasibility of editing a specific variant via different approaches, e.g. homology directed repair (HDR), base editing or prime editing.

Binding of said agent can result in depletion of the cell expressing the first isoform of CD117. Various mechanisms can lead to cell depletion. Antibody dependent cellular cytotoxicity (ADCC) results from binding of the agent to a target protein and activation of NK cells through the Fc part on the agent bound by an FcR expressed by, e.g., NK cells. The Fc part of an immunoglobulin refers to the C-terminal region of an immunoglobulin heavy chain. The Fc part can be wildtype or engineered. Mutations of enhanced, engineered Fc parts are known in the art. For certain therapeutic situations, it is desirable to reduce or abolish the normal binding of the wild-type Fc region of an antibody, such as of a wild-type IgG Fc region to one or more or all of Fc receptors and/or binding to a complement component, such as C1 q in order to reduce or abolish the ability of the antibody to induce effector function. For instance, it may be desirable to reduce or abolish the binding of the Fc region of an antibody to one or more or all of the Fcγ receptors, such as: FcγRI, FcγRIIa, FcγRIIb, FcγRIIIa. Effector function can include, but is not limited to, one or more of the following: complement dependent cytotoxicity (CDC), antibody-dependent cell-mediated cytotoxicity (ADCC), antibody-dependent cellular phagocytosis (ADCP), cytokine secretion, immune complex-mediated antigen uptake by antigen-presenting cells, binding to NK cells, binding to macrophages, binding to monocytes, binding to polymorphonuclear cells, direct signaling inducing apoptosis, crosslinking of target-bound antibodies, dendritic cell maturation, or T cell priming. Binding of said agent may also lead to the blocking of binding of the natural receptor ligand and thereby result in cell death and apoptosis without cell-mediated depletion.

A reduced or abolished binding of an Fc region to an Fc receptor and/or to C1 q is typically achieved by mutating a wild-type Fc region, such as of an IgG1 Fc region, more particular a human IgG1 Fc region, resulting in a variant or engineered Fc region of said wild-type Fc region, e.g., a variant human IgG1 Fc region. Substitutions that result in reduced binding can be useful. For reducing or abolishing the binding properties of an Fc region to an Fc receptor, non-conservative amino acid substitutions, i.e. replacing one amino acid with another amino acid having different structural and/or chemical properties and/or charges, are preferred.

In certain embodiments of the present disclosure the Fc region of the antibody is of the IgG1 isotype, carrying the LALA or PG-LALA mutations, i.e. the constant region carries a L234A, a L235A and a P329G mutation or PA-LALA mutations, i.e. the constant region carries a L234A, a L235A and a P329A mutation, or a AEASS, i.e. the constant region carries a L234A, a L235A and a P329A mutation or a L234A, a L235E, G237A, A330S and a P331S mutation. The skilled person will be aware of possibilities to engineer the Fc region to obtain a desired effect.

Surrogate ADCC assays constitute an industry standard to quantitate an agent's potency to mediate ADCC as described in the experimental part. Engineered Jurkat reporter cells carry an NFAT-responsive luciferase gene and an Fc receptor, such as human FcgRIIIa. Binding of the Fc receptor to bound antibody results in NFAT induction through receptor clustering and therefore a luciferase signal. Absence of binding and therefore clustering does not result in a luciferase signal. Cells expressing either no target protein (e.g. HEK or DF-1 cells or human myeloid cancer cells such as TF-1, KG-1 or KASUMI-1 with a CD117 knock-out), the wildtype protein (e.g. HEK-CD117 of DF-1-CD117, or TF-1, KG-1 or KASUMI-1 cell lines) or individual variants (e.g. CD117 variants) were incubated with the test agent (e.g. antibody Refmab #1) and mixed with the ADCC reporter cells. Then luciferase was measured to quantify the ADCC signal. The luciferase luminescence signals were normalized to the maximal signal observed in HEK-CD117, DF-1-CD117 or the corresponding myeloid cancer cell line. ADCC was measured with an ADCC Reporter Assay (Promega, Cat. No. G7015).

Other potential modes-of-action in line with the present disclosure are possible as well. This includes antibody-mediated displacement of SCF, dimerization blockers or antibody binding outside any of these regions, for example via the use of an ADC. An alternative way of depleting target cells is through the use of T cell engager molecules. For example, a bispecific T cell engager using a CD117 binding site derived from antibody Refmab #1 and a CD3 (OKT3) binding site may be used. The same target cells as used for the ADCC assay are used. Primary human T cells and the bispecific T cell engager are added. Activation of human T cells was quantified by FACS by determining the frequency of CD69 upregulation.

The depleting agent according to the present disclosure binds specifically to one isoform of CD117 and allows the depletion of cells expressing said isoform.

More preferably, in specific embodiments, said depleting agent according to the present disclosure does not bind or binds substantially weaker to a first isoform of CD117 but binds specifically to a second isoform of CD117 and allows the depletion of said cells expressing said second isoform of CD117, in particular in methods of use as disclosed herein. In particular, said depleting agent which does not bind or binds substantially weaker to a first isoform of CD117 but binds specifically to a second isoform of CD117 expressed in patient's cell is used to deplete patient's cells but not hematopoietic stem cells or their progeny expressing said first isoform of CD117 transplanted to restore hematopoiesis in said patient.

In another specific embodiments, said depleting agent according to the present disclosure does not bind or binds substantially weaker to a second isoform of CD117 but binds specifically to a first isoform of CD117 and allows the depletion of cells expressing said first isoform of CD117, in particular in methods of use as disclosed herein. In particular, said depleting agent which does not bind or binds substantially weaker to a second isoform of CD117 but binds specifically to a first isoform of CD117 expressed in transplanted cells is used to deplete specifically transplanted cells to avoid eventual severe side effects such as graft-versus-host disease due to transplantation.

Selective depletion of cells expressing a specific isoform of CD117 can be achieved without limitation by complement-dependent cytotoxicity (CDC), antibody-dependent cellular cytotoxicity (ADCC) or antibody-dependent cellular phagocytosis (ADCP).

In certain embodiments, the antigen binding region is coupled to an effector compound such as a drug or a toxin. Such conjugates are referred to herein as “immunoconjugates”, “antibody-drug conjugates” or “ADCs”. A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, maytansinoids, calicheamicins, indolinobenzodiazepines, pyrolobenzodiazepines, pyrridinobenzodiazepines, camptothecins, topotecan, irinotecan, belotecan, deruxtecan, alpha-amanitin, microcystins, auristatins and puromycin and analogs or homologs thereof.

In another particular embodiment, said depleting agent is an immune cell harboring an antigen receptor such as a chimeric antigen receptor (CAR). See for example Myburgh et al. Leukemia (2020) 34:2688-703. Said immune cell may express a recombinant antigen binding region, also named antigen receptor on its cell surface. By “recombinant” is meant an antigen binding region which is not encoded by the cell in its native state, i.e. it is heterologous, non-endogenous. Expression of the recombinant antigen binding region can thus be seen to introduce a new antigen specificity to the immune cell, causing the cell to recognise and bind a previously unrecognised antigen. The antigen receptor may be isolated from any useful source. In certain embodiments of the present disclosure said cell comprising an antigen binding region is a CAR T-cell, a CAR NK cell, CAR Treg or a CAR macrophage. In a preferred embodiment of the present disclosure said cell comprising an antigen binding region is a CAR T-cell. In another preferred embodiment of the present disclosure said cell comprising an antigen binding region is a primary T cell comprising a CAR.

In a particular embodiment, said recombinant antigen receptor is a chimeric antigen receptor (CAR). CARs are fusion proteins comprising an antigen-binding region, typically derived from an antibody, linked to the signaling domain of the TCR complex. CARs can be used to direct immune cells such T-cells or NK cells against a target antigen if a suitable antigen-binding region is selected.

The antigen-binding region of a CAR is typically based on a scFv (single chain variable fragment) derived from an antibody. In addition to an N-terminal, extracellular antibody-binding region, CARs typically may comprise a hinge domain, which functions as a spacer to extend the antigen-binding region away from the plasma membrane of the immune effector cell on which it is expressed, a transmembrane (TM) domain, an intracellular signaling domain (e.g. the signaling domain from the zeta chain of the CD3 molecule (CD37) of the TCR complex, or an equivalent) and optionally one or more co-stimulatory domains which may assist in signaling or functionality of the cell expressing the CAR. Signaling domains from co-stimulatory molecules including CD28, OX-40 (CD134), and 4-1BB (CD137) can be added alone (second generation) or in combination (third generation) to enhance survival and increase proliferation of CAR modified immune cells.

The skilled person is able to select an appropriate antigen binding region as described above with which to redirect an immune cell to be used according to the disclosure. In a particular embodiment, the immune cell for use in the method of the disclosure is a redirected T-cell, e.g. a redirected CD8+ T-cell or a redirected CD4+ T-cell, or a redirected NK cell.

Methods by which immune cells can be genetically modified to express a recombinant antigen binding region are well known in the art. A nucleic acid molecule encoding the antigen receptor may be introduced into the cell in the form of e.g. a vector, or any other suitable nucleic acid construct, or by inserting the nucleic acid molecule into the genome using genome editing technologies. Vectors, and their required components, are well known in the art. Nucleic acid molecules encoding antigen binding region can be generated using any method known in the art, e.g. molecular cloning using PCR. Antigen binding region sequences can be modified using commonly used methods, such as site-directed mutagenesis.

### CD117

CD117 (UniProt: P10721; also known as KIT, c-Kit or SCFR) is a cytokine receptor expressed on the surface of hematopoietic stem cells as well as other cell types. CD117 is a receptor tyrosine kinase type III, which binds to SCF (UniProt: P21583; also known as stem cell factor Kit ligand or Mast cell growth factor). Binding of CD117 to SCF leads to the formation of a dimer which activates tyrosine kinase activity, thereby activating signal transduction molecules that propagate the signal into the cell. Signaling through CD117 plays a role in cell survival, proliferation, and differentiation.

Human CD117 has the following amino acid sequence (SEQ ID No. 1):

MRGARGAWDFLCVLLLLLRVQTGSSQPSVSPGEPSPPSIHPGKSDLIVRVGDEIRLLCTD
PGFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAYH
RLCLHCSVDQEGKSVLSEKFILKVRPAFKAVPVVSVSKASYLLREGEEFTVTCTIKDVSS
SVYSTWKRENSQTKLQEKYNSWHHGDFNYERQATLTISSARVNDSGVFMCYANNTFGSAN
VTTTLEVVDKGFINIFPMINTTVFVNDGENVDLIVEYEAFPKPEHQQWIYMNRTFTDKWE
DYPKSENESNIRYVSELHLTRLKGTEGGTYTFLVSNSDVNAAIAFNVYVNTKPEILTYDR
LVNGMLQCVAAGFPEPTIDWYFCPGTEQRCSASVLPVDVQTLNSSGPPFGKLVVQSSIDS
SAFKHNGTVECKAYNDVGKTSAYFNFAFKGNNKEQIHPHTLFTPLLIGFVIVAGMMCIIV
MILTYKYLQKPMYEVQWKVVEEINGNNYVYIDPTQLPYDHKWEFPRNRLSFGKTLGAGAF
GKVVEATAYGLIKSDAAMTVAVKMLKPSAHLTEREALMSELKVLSYLGNHMNIVNLLGAC
TIGGPTLVITEYCCYGDLLNFLRRKRDSFICSKQEDHAEAALYKNLLHSKESSCSDSTNE
YMDMKPGVSYVVPTKADKRRSVRIGSYIERDVTPAIMEDDELALDLEDLLSFSYQVAKGM
AFLASKNCIHRDLAARNILLTHGRITKICDFGLARDIKNDSNYVVKGNARLPVKWMAPES
IFNCVYTFESDVWSYGIFLWELFSLGSSPYPGMPVDSKFYKMIKEGFRMLSPEHAPAEMY
DIMKTCWDADPLKRPTFKQIVQLIEKQISESTNHIYSNLANCSPNRQKPVVDHSVRINSV
GSTASSSQPLLVHDDV

Human SCF has the following amino acid sequence (SEQ ID No. 2):

MKKTQTWILTCIYLQLLLFNPLVKTEGICRNRVTNNVKDVTKLVANLPK
DYMITLKYVPGMDVLPSHCWISEMVVQLSDSLTDLLDKFSNISEGLSNY
SIIDKLVNIVDDLVECVKENSSKDLKKSFKSPEPRLFTPEEFFRIENRS
IDAFKDFVVASETSDCVVSSTLSPEKDSRVSVTKPFMLPPVAASSLRND
SSSSNRKAKNPPGDSSLHWAAMALPALFSLIIGFAFGALYWKKRQPSLT
RAVENIQINEEDNEISMLQEKEREFQEV

In a particular embodiment, said surface protein is CD117. In other embodiments said surface protein is CD117 comprising the amino acid sequence of SEQ ID No. 1. In other embodiments said surface protein is CD117 consisting of the amino acid sequence of SEQ ID No. 1.

In certain embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and preferably wherein said first and second isoforms are functional.

In certain embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoforms of said surface protein, wherein said first and second isoforms are functional.

In certain embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoforms of said surface protein, wherein said first and second isoforms are substantially functionally identical.

Several functions are reported for CD117. In certain embodiments the present disclosure related to a first and a second isoform of CD117 wherein both isoforms are functional. In certain embodiments the present disclosure related to a first and a second isoform of CD117 wherein both isoforms are functional indistinguishable. In the present invention, “functionally indistinguishable” refers to a first and a second isoform of CD117 that are equally capable of performing the same function within a cell without significant impairment. In other words, the first and the second isoform are functionally largely indistinguishable. A slight functional impairment may be acceptable. In a preferred embodiment, said first isoform of CD117 remains functional and retain the capacity of performing the same function as the corresponding wildtype isoform within a cell without significant impairment.

One function of CD117 is binding to SCF. Therefore, in certain embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and second isoforms bind to SCF.

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and second isoforms bind to SCF to a substantially similar degree.

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoforms bind to a polypeptide comprising SEQ ID No. 2.

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoforms bind to a polypeptide consisting of SEQ ID No. 2.

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoforms bind to a polypeptide comprising SEQ ID No. 2 in a substantially similar manner. In other embodiments said polypeptide is a polypeptide consisting of SEQ ID No. 2.

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoform bind to a polypeptide comprising SEQ ID No 2 with a Kp which differs by no more than 60%, no more than 50%, no more than 40%, preferably no more than 30%, more preferably no more than 20% and even more preferably no more than 10%. In other embodiments said polypeptide is a polypeptide consisting of SEQ ID No 2.

Binding of CD117 to SCF can be tested by the skilled person by any commonly used assay, such as a FACS assay titrating and measuring the binding of biotinylated SCF and fluorescence-labeled streptavidin to the cells. An example of such an assay is described in Example 8.

Another function of CD117 is SCF-dependent proliferation. Therefore, in certain embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and second isoforms lead to SCF-dependent proliferation. These cells show increased proliferation upon the addition of SCF. This increase in proliferation is measured after 2-5 days using agents to quantify cell viability such as Cell Titer Glow (Promega).

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoforms lead to SCF-dependent proliferation in a substantially similar manner. This can be tested in SCF-dependent cell lines, such as TF-1, or in HSCs. An example of such an assay is described in Example 9. In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoforms lead to SCF-dependent proliferation which differs by no more than 40%, preferably no more than 30%, more preferably no more than 20% and even more preferably no more than 10%. In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoforms lead to SCF-dependent proliferation, wherein said first isoform leads to a loss in binding affinity (KD) which is not larger than 4-fold, preferably not larger than 3-fold and more preferably not larger than 2-fold compared to the second isoform. In another embodiment said first isoform leads to a loss in EC50 of no more than 40%, more preferably no more than 35% and even more preferably no more than 30% compared to the second isoform as measured by FACS.

SCF-dependent proliferation can be tested by the skilled person by any commonly used assay, such as cell count, colony-forming unit (CFU) assay, cell titer glow (CTG; Promega) and carboxyfluorescein succinimidyl ester (CFSE) cell proliferation assay. For the cell count assay, non-modified HSC cultured in liquid medium in presence of anti-CD117 antibody will show a strong reduction in proliferation of cell number over time compared to genetically modified HSC expressing the isoform of CD117. The 14-days CFU assay assess the hematopoietic potential of HSCs, i.e. their ability to give rise to differentiated colonies, in a semi-solid medium. Non-modified HSCs cultured in presence of anti-CD117 antibody are unable to form hematopoietic colonies while genetically modified HSC are protected against the anti-CD117 antibody and give rise to colonies. When cultured in absence of anti-CD117 antibody, genetically modified HSC demonstrate similar proliferation than non-modified HSCs in liquid or semi-solid culture, thus confirming the retention of the normal function of CD117 isoform.

In certain embodiment said first isoform leads to a loss of maximum proliferation of no more than 40%, more preferably no more than 35% and even more preferably no more than 30% compared to the second isoform as measured via EC50 by FACS.

Another function of CD117 is SCF-dependent phosphorylation. Therefore, in certain embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and second isoforms lead to phosphorylation upon addition of SCF to the cell culture medium (SCF-dependent phosphorylation).

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoforms lead to SCF-dependent phosphorylation in a substantially similar manner.

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein said first and second isoforms lead to SCF-dependent phosphorylation which differs by no more that 30%, preferably no more than 20% and even more preferably no more than 10%. In other embodiments the reduction of SCF-dependent phosphorylation of the first isoform is less than 50% as compared to the second isoform.

SCF-dependent phosphorylation can be tested by the skilled person by any commonly used assay, such as an enzyme-linked immunoassay (ELISA), FACS or Western Blot. Cells expressing CD117 are incubated with SCF for different time points (5-15 min), before collection of cells and freezing in cell lysis buffer. Upon cell lysis through flash freezing in liquid nitrogen and thawing at 37° C., the amount of phosphorylated CD117 at position Tyr719 is assessed using ELISA or Western Blot methods specifically known to detect phosphorylated Tyr719 of CD117 as well as total level of CD117 as a control.

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein the KD of the first isoform of CD117 to SCF is less than four-fold, preferably less than three-fold, more preferably less than two-fold and most preferably less than 1.5-fold higher, than the KD of the second isoform of CD117 to SCF. Preferably said second isoform is wildtype CD117.

In other embodiments the present disclosure relates to a mammalian cell or a population of cells expressing a first isoform of the surface protein CD117 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of CD117 and wherein the KD of the first isoform of CD117 to SCF is less than 0.25-fold, preferably less than 0.33-fold, more preferably less than 0.5-fold and most preferably less than 0.66-fold lower, than the KD of the second isoform of CD117 to SCF.

In line with the present disclosure, it is also possible to combine additional variants or isoforms of CD117 within the methods and compositions of the present disclosure. Such isoforms may for example include double mutants. Such isoforms may for example also include single and double mutants. The methods and compositions of the present disclosure may also be combined with cells carrying a CD117 knock out, e.g., a permanent knock out or a temporarily knock out (e.g. via CRISPRoff). The methods and compositions of the present disclosure may also be used in the depletion of myeloid cells in solid tumors in order to enhance tumor responses.

The methods and compositions of the present disclosure may also be combined with cells combinations, in particular when said surface protein is CD117 with knock out of other targets, such as CD45, CD123, CD33, CD7, CLEC12A, CD44, FLT3, CD300F, EVI2B, TPO and combination thereof.

The methods and compositions of the present disclosure may also comprise cells expressing first isoform of CD117 (CD117 variants) and other surface protein variants such as CD123 variants, CD33 variants, CD7 variants, CLEC12A variants, CD45 variants FLT3 variants, CD300F variants, EVI2B variants, TPO variants and any combination thereof.

### Polymorphism of CD117

The cell expressing the first isoform of CD117 according to the present disclosure comprises genomic DNA with at least one polymorphic allele in the nucleic acid encoding said CD117 isoform. In particular, said polymorphism induces at least one mutation involved in the binding of a specific agent in comparison to said second isoform.

Said polymorphism is preferably within a nucleic acid sequence encoding the surface protein region of CD117 involved in binding of the first agent, preferably located in the extracellular portion of CD117, in particular in a solvent-exposed secondary structure element. More particularly, said polymorphism is within a nucleic acid sequence encoding at least one specific amino acid residue involved in binding of the first agent. Said polymorphism can be a mutation such as a deletion, a substitution, an insertion, or a combination thereof of at least 1, 2, 3, 4, 5, 6, 7, 8, 9, 10, 12, 15 or 20 nucleotides. In a particular embodiment, said polymorphism is a single nucleotide polymorphism.

The difference in the sequence of the two isoforms may also be genetically introduced. Also here the sequence difference is preferably within a nucleic acid sequence encoding the CD117 region involved in binding of the first agent, preferably located in the extracellular portion of said surface protein, in particular in a solvent-exposed secondary structure element. More particularly, said sequence difference is within a nucleic acid sequence encoding at least one specific amino acid residue involved in binding of the first agent. Said sequence difference can be a mutation such as a deletion, a substitution, an insertion or a combination thereof of at least 1, 2, 3, 4, 5, 6, 7, 8, 9, 10, 12, 15 or 20 nucleotides. In a particular embodiment, said sequence difference is a single point mutation.

The present disclosure provides polymorphisms in CD117, including in particular polymorphisms including substitution of the residues E73, T74, V120, D121, R122, S123, Y125, K127, K193, I201, K203, S239, Y259, N260, S261, D266, Y269 or R271. In certain embodiments, the present disclosure provides polymorphisms in CD117, including in particular polymorphisms including substitution of the residues E73, V120, D121, R122, S123, K127, K193, S239, Y259 or S261. In certain embodiments, the present disclosure provides polymorphisms in CD117, including in particular polymorphisms including substitution of the residues E73, D121, R122, S123, S239, Y259 or S261. In other embodiments, the present disclosure provides polymorphisms in CD117, including in particular polymorphisms including substitution of the residues E73, D121 or S123. Particular preferred polymorphisms include substitutions of the residue E73, wherein E73 is substituted with an amino acid selected from the group consisting of K, L, Q, G, Y and R.

Preferably said substitution is E73K, E73Y, or E73R. Other preferred polymorphisms include substitutions of the residue D121, wherein D121 is substituted with an amino acid selected from the group consisting of S, V, Y, H, K, R and T. Preferably said substitution is D121Y, D121H, D121K, D121R or D121T. Most preferably said substitution is D121H or D121K. Other preferred polymorphisms include substitutions of the residue S123, wherein S123 is substituted with P, F or K. Most preferably said substitution is S123K. Other preferred polymorphisms include substitutions of the residue S239, wherein S239 is substituted with H or K. Other preferred polymorphisms include substitutions of the residue K193, wherein K193 is substituted with G, T, M, D or E. Yet other preferred polymorphisms include substitutions of the residue Y259, wherein Y259 is substituted with an amino acid selected from the group consisting of E, A, G, P, C and H. Preferably said substitution is Y259P, Y259A or Y259G. Most preferably said substitution is Y259A.

In certain embodiments, the present disclosure provides a variant of CD117, wherein said variant of CD117 comprises an amino acid sequence of SEQ ID No. 1, wherein one or more of the amino acids is substituted selected from the group consisting of E73, T74, V120, D121, R122, S123, Y125, K127, K193, I201, K203, S239, Y259, N260, S261, D266, Y269 and R271, preferably selected from the group consisting of E73, V120, D121, R122, S123, K127, K193, S239, Y259 and S261, more preferably selected from the group consisting of E73, D121, R122, S123, S239, Y259 and S261, and most preferably selected from the group consisting of E73, D121 and S123. In certain preferred embodiments said amino acid is E73. In other preferred embodiments said amino acid is D121. In other preferred embodiments said amino acid is S123. In other preferred embodiments said amino acid is S239. In other preferred embodiments said amino acid is Y259.

In certain embodiments, the present disclosure provides a variant of CD117, wherein said variant of CD117 comprises an amino acid sequence of SEQ ID No. 1, wherein residue E73 is substituted with an amino acid selected from the group consisting of K, L, Q, G, Y and R, preferably E73K, E73Y, or E73R. In certain embodiments, the present disclosure provides a variant of CD117, wherein said variant of CD117 comprises an amino acid sequence of SEQ ID No. 1, wherein residue D121 is substituted with an amino acid selected from the group consisting of S, V, Y, H, K, R and T, preferably Y, H, K, R or T, and most preferably H or K. In certain embodiments, the present disclosure provides a variant of CD117, wherein said variant of CD117 comprises an amino acid sequence of SEQ ID No. 1, wherein residue S123 is substituted with P, F or K, most preferably with K. In certain embodiments, the present disclosure provides a variant of CD117, wherein said variant of CD117 comprises an amino acid sequence of SEQ ID No. 1, wherein residue S239 is substituted with H or K. In certain embodiments, the present disclosure provides a variant of CD117, wherein said variant of CD117 comprises an amino acid sequence of SEQ ID No. 1, wherein residue K193 is substituted with G, T, M, D or E. In certain embodiments, the present disclosure provides a variant of CD117, wherein said variant of CD117 comprises an amino acid sequence of SEQ ID No. 1, wherein residue Y259 is substituted with an amino acid selected from the group consisting of E, A, G, P, C and H, preferably P, A or G, and most preferably A.

In certain embodiments, the present disclosure relates to a mammalian cell or a population of cells expressing one of aforementioned variants of CD117.

It will be appreciated that amino acid may be designated by the 3-letter code or the 1-letter code, which both are familiar to the skilled person.

Table 1 shows the 20 natural occurring amino acids.

TABLE 1

| | Three | One |
|---|---|---|
| | letter | letter |
| Amino acid | code | code |
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

### Natural Polymorphism

In a particular embodiment, said cell according to the present disclosure is selected from a subject comprising native genomic DNA with at least one natural polymorphism allele, preferably single nucleotide polymorphism (SNP) in the nucleic acid encoding said isoform.

In a particular embodiment, cells are selected from a subject that comprises native genomic DNA with at least one natural polymorphism allele, in particular SNP, in a nucleic acid sequence encoding CD117 region involved in anti-CD117 agent binding, preferably located in the extracellular portion of said surface protein, more preferably in a solvent-5 exposed secondary structure element.

Certain naturally occurring SNPs are described in the literature. These naturally SNPs may be used within the spirit of the present disclosure with a respective binding agent which is able to discriminate such SNP from another isoform of CD117.

Some naturally occurring SNPs of human CD117 are shown in Table 2. A list of natural occurring SNPs can also be found on the gnomAD database: https://gnomad.broadinstitute.org/gene/ENSG00000157404?dataset=gnomad_r2_1

TABLE 2
Identifier

| SNP Identifier | Amino acid | Amino acid | |
|---|---|---|---|
| (UniProt) | position | change | Reference |
| VAR_004104 | 583 | E → K | J. Clin. Invest. 89:1713-1717(1992) |
| VAR_033129 | 584 | F → C | Am. J. Med. Genet. 95:79-81(2000) |
| VAR_004105 | 584 | F → L | Am. J. Hum. Genet. 50:261-269(1992) |
| VAR_033130 | 601 | G → R | Am. J. Med. Genet. 95:79-81(2000) |
| VAR_033131 | 656 | L → P | Am. J. Med. Genet. 95:79-81(2000) |
| VAR_004106 | 664 | G → R | Proc. Natl. Acad. Sci. U.S.A. 88:8696-8699(1991) |
| VAR_004107 | 791 | R → G | J. Invest. Dermatol. 101:22-25(1993) |
| VAR_033132 | 796 | R → G | Am. J. Med. Genet. 75:101-103(1998) |
| VAR_004108 | 812 | G → V | J. Invest. Dermatol. 101:22-25(1993) |
| VAR_033137 | 847 | T → P | J. Invest. Dermatol. 111:337-338(1998) |
| VAR_004110 | 893-896 | deletion | Hum. Mutat. 6:343-345(1995) |

### Gene Editing

In another particular embodiment, said cell expressing the first isoform of CD117 according to the present disclosure is obtained by gene editing, preferably by changing the sequence encoding said surface protein in the patient's native genomic DNA.

The cell can be genetically engineered by introducing into the cell a gene editing system to induce said polymorphism resulting in insertion, deletion and/or substitution of amino acids of the surface protein. Said gene editing modality targets a nucleic acid sequence, named herein target sequence encoding surface protein region involved in first agent binding as described above. In particular, when said surface protein is CD117, said gene editing modality targets a nucleic acid encoding at least one amino acid residue in position E73, T74, V120, D121, R122, S123, Y125, K127, K193, 201, K203, S239, Y259, N260, S261, D266, Y269 or R271 of SEQ ID NO: 1. Preferably amino residue E73 is substituted by an amino acid selected from the group consisting of K, L, Q, G, Y and R, preferably selected from the group consisting of K, Y, and R. Also preferably, amino acid residue D121 is substituted with S, V, Y, H, K, R or T, preferably Y, H, K, R or T, and most preferably H or K. Also preferably, amino acid residue S123 is substituted with P, F or K, most preferably K. Also preferably, amino acid residue K193 is substituted with G, T, M, D or E. Also preferably, amino acid residue S239 is substituted with H or K. Also preferably, amino acid residue Y259 is substituted with E, A, G, P, C or H, more preferably P, A or G, and most preferably A.

Gene editing enzymes may be sequence-specific nucleases, base editors, prime editors or CRISPR-transposon based systems.

The term “nuclease” refers to a wild type or variant enzyme capable of catalyzing the hydrolysis (cleavage) of phosphodiester bonds between nucleotides of a nucleic acid (DNA or RNA) molecule, preferably a DNA molecule. By “cleavage” is intended a double-strand break or a single-strand break event.

The term “sequence-specific nuclease” refers to a nuclease which cleaves nucleic acid in a sequence-specific manner. Different types of site-specific nucleases can be used, such as Meganucleases, TAL-nucleases (TALEN), Zing-finger nucleases (ZFN), or RNA/DNA guided endonucleases like Clustered Regularly Interspaced Short Palindromic Repeats (CRISPR)/Cas system and Argonaute (Review in Li et al., Nature Signal transduction and targeted Therapy, 5, 2020; Guha et al., Computational and Structural Biotechnology Journal, 2017, 15, 146-160).

According to the present disclosure, the nuclease generates a DNA cleavage within a target sequence, said target sequence encodes a surface protein region involved in first agent binding as described above. In particular embodiments, the inventors use CRISPR system to induce a cleavage within a target sequence encoding surface protein region recognized by first agent as described above.

By “target sequence”, it is intended targeting a part of the sequence encoding the region on CD117 involved in first agent binding as described as described above and/or sequences adjacent to said region on CD117 involved in first agent binding, in particular at least one (one or two) sequence of up to 50 nucleotides adjacent to said region on CD117 involved in first agent binding, preferably 20, 15, 10, 9, 8, 7, 6 or 5 nucleotides adjacent to said agent binding site.

CRISPR system involves two or more components, Cas protein (CRISPR-associated protein) and a guide RNA. The guide RNA can be a single guide RNA or a dual guide RNA. Cas protein is a DNA endonuclease that uses guide RNA sequence as a guide to recognize and generate double-strand cleavage in DNA that is complementary to the target sequence. Cas systems that generate single strand breaks require only one nuclease domain. Cas systems that generate double strand breaks require two nuclease domains. Cas protein may comprise two active cutting sites, such as HNH nuclease domain and RuvC-like nuclease domain.

By Cas protein is also meant an engineered endonuclease, homologue or orthologue of Cas 9 which is capable of cleaving target nucleic acid sequence. In particular embodiments, Cas protein may induce a cleavage in the nucleic acid target sequence which can correspond to either a double-stranded break or a single-stranded break. Cas protein variant may be a Cas endonuclease that does not naturally exist in nature and that is obtained by protein engineering or by random mutagenesis. The Cas protein can be one type of the Cas proteins known in the art. Non-limiting examples of Cas proteins include Cas1, Cas1B, Cas2, Cas3, Cas4, Cas5, Cas6, Cas7, Cas8, Cas9 (also known as Csn1 and Csx12), SaCas9, Cas12, Cas12a (Cpf1), CasIO, Csy1, Csy2, Csy3, Cse1, Cse2, Csc1, Csc2, Csa5, Csn2, Csm2, Csm3, Csm4, Csm5, Cmrl, Cmr3, Cmr4, Cmr5, Cnrr6, CsbI, Csb2, Csb3, Csx17, CsxM, Csx IO, Cs I6, CsaX, Csx3, Cs I, Csx15, Csf1, Csf2, CsO, Csf4, homologs, orthologs thereof, or modified versions thereof. Preferably Cas protein is Streptococcus pyogenes Cas 9 protein.

Cas is contacted with a guide RNA (gRNA) designed to comprise a complementary sequence to the target sequence to specifically induce DNA cleavage within said target sequence, in particular according to the present disclosure a complementary sequence of a part of target sequence encoding surface protein region recognized by agent as described above.

As used herein, a “guide RNA”, “gRNA”, “sgRNA” or “single guide RNA” refers to a nucleic acid that promotes the specific targeting or homing of a gRNA/Cas complex to a target nucleic acid.

In particular, gRNA refers to RNA that comprises a transactivating crRNA (tracrRNA) and a crRNA. Preferably, said guide RNA corresponds to a crRNA and tracrRNA which can be used separately or fused together to generate a single guide RNA. The complementary sequence pairing with the target sequence recruits Cas to bind and cleave the DNA at the target sequence.

According to the present disclosure, crRNA is engineered to comprise a complementary sequence to a part of a target sequence as described above encoding surface protein region recognized by agent, such that it is capable of targeting said region. In a preferred embodiment sgRNA is used to target the binding site of the said binding agent. In another preferred embodiment, the guide RNA contains chemically modifications known to the person skilled in the art.

In a particular embodiment, the crRNA comprises a sequence of 5 to 50 nucleotides, preferably 15 to 30 nucleotides, more preferably 20 nucleotides which is complementary to the target sequence. As used herein, the terms “complementary sequence” refers to the sequence part of a polynucleotide (e.g. part of crRNA or tracRNA) that can hybridize to another part of polynucleotides under standard low stringent conditions. Preferentially, the sequences are complementary to each other pursuant to the complementarity between two nucleic acid strands relying on Watson-Crick base pairing between the strands, i.e. the inherent base pairing between adenine and thymine (A-T) nucleotides and guanine and cytosine (G-C) nucleotides. Said gRNA can be designed by any methods known by one of skill in the art in view of the present disclosure.

According to the present disclosure said target sequence encodes surface protein region on CD117 involved in first agent binding, preferably located in the extracellular portion of CD117, more preferably in an extracellular loop in comparison to said second isoform, again more preferably comprising amino acid residues involved in agent binding.

In a preferred embodiment, when surface protein is CD117, said target sequence encodes a CD117 region involved in binding of a first agent, such as anti-CD117 agent binding as disclosed above. Preferably said target sequence encodes at least one residue in position E73, T74, V120, D121, R122, S123, Y125, K127, K193, I201, K203, S239, Y259, N260, S261, D266, Y269 or R271 of SEQ ID NO: 1.

In a particular embodiment, said crRNA may target a sequence encoding a CD117 region involved in binding of a first agent and particularly comprise one of the sequences shown in the Table 3 (crRNA sequence).

TABLE 3

| gRNA | Sequence | SEQ ID NO: |
|---|---|---|
| KIT_E73_1 | CTGATCCGGGCTTTGTCAAA | 3 |
| KIT_E73_2 | AAAGTCCATTTGACAAAGCC | 4 |
| KIT_E73_3 | CAAATGGACTTTTGAGATCC | 5 |
| KIT_E73_4 | TATTCTCATTCGTTTCATCC | 6 |
| KIT_E73_5 | ATGAGAATAAGCAGAATGAA | 7 |
| KIT_E73_6 | TAAGCAGAATGAATGGATCA | 8 |
| KIT_120_123_1 | GTTGTCTTCTTTCCCATACA | 9 |
| KIT_120_123_2 | CTTCTTTCCCATACAAGGAG | 10 |
| KIT_120_123_3 | TTGTTGACCGCTCCTTGTAT | 11 |
| KIT_120_123_4 | CTTGTTGACCGCTCCTTGTA | 12 |
| KIT_120_123_5 | CATACAAGGAGCGGTCAACA | 13 |
| KIT_120_123_6 | GCGGTCAACAAGGAAAAGCT | 14 |

In another terms, when the surface protein is CD117, said nucleic acid construct preferably can comprise:
a gRNA sequence of: SEQ ID NO: 3 to 8 which targets a sequence encoding amino acid residue E73 of SEQ ID NO: 1, or
a gRNA sequence selected from the group consisting of: SEQ ID NO: 9 to 14 which targets a sequence encoding amino acid residues V120, D121, R122, S123 of SEQ ID NO: 1.

In other particular embodiment gene editing is performed via HDR and the HDR template may comprise one of the sequences shown in Table 4:

| HDR | | SEQ ID |
|---|---|---|
| template | Sequence | NO: |
| E73K- | CTGATCCGGGCTTTGTCAAATGGACTTTTGAGATATTGGATAAAACGAATGAGAA | 55 |
| CIT03_2- | TAAGCAGAATGAATGGATCACGGAAAAGG | |
| plus | | |
| E73K- | CCTTTTCCGTGATCCATTCATTCTGCTTATTCTCATTCGTTTTATCCAATATCTCAAA | 56 |
| CIT03_2- | AGTCCATTTGACAAAGCCCGGATCAG | |
| min | | |
| E73K- | GTGGGCGACGAGATTAGGCTGTTATGCACTGATCCGGGCTTTGTCAAATGGACTT | 57 |
| CIT03_2- | TTGAGATATTGGATAAAACGAATGAGAATAAGCAGAATGAATGGATCACGGAAA | |
| plus_140 | AGGCAGAAGCCACCAACACCGGCAAATACAC | |
| bp | | |
| E73K- | GTGTATTTGCCGGTGTTGGTGGCTTCTGCCTTTTCCGTGATCCATTCATTCTGCTTA | 58 |
| CIT03_2- | TTCTCATTCGTTTTATCCAATATCTCAAAAGTCCATTTGACAAAGCCCGGATCAGT | |
| min 140 | GCATAACAGCCTAATCTCGTCGCCCAC | |
| bp | | |

The DNA strand break that is introduced by the nuclease according to the disclosure can result in mutation of the DNA at the cleavage site via non-homologous end joining (NHEJ) which often results in small insertions and/or deletions or replacement of the DNA surrounding the cleavage site via homology-directed repair (HDR).

In a preferred embodiment, said polymorphism within nucleic acid encoding the isoform of CD117 is induced via HDR repair following the DNA cleavage and the introduction of an exogeneous nucleotide sequence, named herein HDR template.

HDR template comprises a first and a second portion of sequence which are homologous in to regions 5′ and 3′ of the target sequence, respectively and a middle sequence portion comprising polymorphism. Following cleavage of the target sequence, a homologous recombination event is achieved between the genome containing the target sequence and the HDR template and the genomic sequence containing the target sequence is replaced by the exogeneous sequence.

Preferably, homologous sequences of at least 20 bp, preferably more than 30 bp, more preferably more than 50 bp and most preferably less than 200 bp are used. Homologous sequences may be dsDNA or ssDNA. Preferably the homologous sequences are ds DNA. Indeed, shared DNA homologies are located in regions flanking upstream and downstream the site of the break and the exogeneous sequence to be introduced should be located between the two arms. The flanking sequences may be symmetrical or asymmetrical. Both strands of the target nucleic acid, i.e. the plus strand or the minus strand, may be targeted. Optionally, a PAM sequence may be used, which may be silenced to improve HDR.

In a preferred embodiment, the cell according to the present disclosure is genetically engineered by introducing into said cell said site-specific nuclease which targets the sequence encoding the region on CD117 recognized by said first agent as described above and a HDR template.

In another particular embodiment, said gene editing enzyme is a DNA base editor as described in Komor et al., Nature 533, 420-424, and in Rees H A, Liu D R. Nat Rev Genet. 2018; 19:770-788, or a prime editor as described in Anzalone et al. Nature, 2019, 576:149-157, Matsoukas et al., Front Genet. (2020) 11:528, Chen et al. Cell (2021) 184:5635-52, Koblan et al, Nat Biotechnol (2021) 39:1414-25 and Kantor A. et al. Int. J. Mol. Sci. 2020, 21 (6240). Base editor or prime editor can be used to introduce mutations at specific sites in the target sequence.

According to the present disclosure, the base editor or prime editor generates a mutation within the target sequence by sequence-specific targeting of the sequence encoding the region on CD117 involved in first agent binding.

In particular, said base editor or prime editor are CRISPR base or prime editors. Said CRISPR base or prime editor may comprise as catalytically inactive sequence specific nuclease a dead Cas protein (dCas). It may also comprise Cas9 with a mutated nuclease domain. dCas refers to a modified Cas nuclease which lacks endonucleolytic activity. Nuclease activity can be inhibited or prevented in dCas proteins by one or more mutations and/or one or more deletions in the HNH and/or RuvC-like catalytic domains of the Cas protein. The resulting dCas protein lacks nuclease activity but bind to a guide RNA (gRNA)-DNA complex with high specificity and efficiency to specific target sequence. In particular embodiment, said dCas may be a Cas nickase wherein one catalytic domain of the Cas is inhibited or prevented.

Said base editor is complexed with a guide RNA (gRNA) designed to comprise a complementary sequence of the target nucleic acid sequence to specifically bind said target sequence as described above.

Said gRNA can be designed by any methods known by one of skill in the art in view of the present disclosure. In a particular embodiment, said gRNA may target the sequence encoding the region on CD117 recognized by said first agent as described above.

As non-limiting examples said base editor is a nucleotide deaminase domain fused to a dead Cas protein, in particular Cas nickase. Said nucleotide deaminase may be an adenosine deaminase or cytidine deaminase. Said nucleotide deaminase may be natural or engineered deaminase.

In a particular embodiment, said base editor may be as non-limiting examples selected from the group consisting of: BE1, BE2, BE3, BE4, HF-BE3, Sa-BE3, Sa-BE4, BE4-Gam, saBE4-Gam, YE1-BE3, EE-BE3, YE2-BE3, YEE-BE3, VQR-BE3, VRER-BE3, SaKKH-BE3, cas12a-BE, Target-AID, Target-AID-NG, xBE3, eA3A-BE3, A3A-BE3, BE-PLUS, TAM, CRIPS-X, ABE7.9, ABE7.10, ABE7.10* xABE, ABESa, ABEmax, ABE8e, VQR-ABE, VRER-ABE and SaKKH-ABE.

Said prime editor consists of a fusion of a catalytically inactive sequence specific nuclease as described above, particularly a Cas nickase and a catalytically active engineered reverse transcriptase (RT) enzyme. Said fusion protein is used in combination with a prime editing guide RNA (pegRNA) which contains the complementary sequence to the target sequence as described above, particularly when surface protein is CD117 comprises one of the sequences described in the Table 3 and also an additional sequence comprising a sequence that binds to the primer binding site region on the DNA. In particular embodiment, said reverse transcriptase enzyme is a Maloney murine leukemia virus RT enzyme and variants thereof. Said prime editor may be as non-limiting examples selected from the group consisting of: PE1, PE2, PE3 and PE3b, or any of the prime editors described in Chen et al. Cell (2021) 184:5635-52 or Koblan et al, Nat Biotechnol (2021) 39:1414-25.

### Anti-CD117 Agents

Several anti-CD117 moieties are known in the art, some of which are currently in development. Antibody SR-1 was originally isolated from a hybridoma (WO1992017505). Humanized versions of SR-1 were generated (WO2007127317; WO2020112687). Anti-CD117 drug conjugates are described in WO2016020791. Other anti-CD117 antibodies are described in WO2015050959 and WO2019084064. Certain anti-CD117 antibodies are also available commercially, e.g. antibody 104D2 Dianova (#117PE-100T). These and other anti-CD117 moieties may be used in the context of the present disclosure. Several anti-CD117 antibodies were also generated in the present disclosure, in full length antibody format, as well as in Fab format. Details are provided in Example 1.

In a particular embodiment, said depleting agent which binds to said second isoform of CD117 and does not bind or binds substantially weaker to said first isoform of CD117 as described above binds specifically to an epitope including the amino acids E73, T74, V120, D121, R122, S123, Y125, K127, K193, I201, K203, S239, Y259, N260, S261, D266, Y269 and/or R271 of SEQ ID NO: 1. More preferably, said depleting agent binds specifically to an epitope including the amino acids E73, V120, D121, R122, S123, K127, K193, S239, Y259 and/or S261 of SEQ ID NO: 1. Even more preferably, said depleting agent binds specifically to an epitope including the amino acids E73, D121, R122, S123, S239, Y259 and/or S261 of SEQ ID NO: 1. Most preferably, said depleting agent binds specifically to an epitope including amino acid E73, D121 and/or S123 of SEQ ID NO: 1.

In a preferred embodiment, said anti-CD117 agent comprises an antigen binding region comprising:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22.

In another embodiment, said anti-CD117 agent comprises an antigen binding region comprising:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22,
   wherein the antigen binding region comprises the following mutations:
the asparagine in the VLCDR1 region (SEQ ID NO: 20) is replaced by glutamic acid, and
the aspartic acid in the VLCDR3 region (SEQ ID NO: 22) is replaced by glutamic acid, and optionally,
the second asparagine in the VLCDR3 region (SEQ ID NO: 22) is replaced by lysine.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22.
   wherein the antigen binding region comprises the following mutations:
the asparagine in the VLCDR1 region (SEQ ID NO: 20) is replaced by glutamic acid, and
the aspartic acid in the VLCDR3 region (SEQ ID NO: 22) is replaced by glutamic acid, and optionally,
the second asparagine in the VLCDR3 region (SEQ ID NO: 22) is replaced by lysine.

In another embodiment, said anti-CD117 agent comprises an antigen binding region comprising:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60.

In a another embodiment, said anti-CD117 agent comprises an antigen binding region comprising:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61.

In another preferred embodiment, said anti-CD117 agent comprises an antigen binding region comprising:
a) an antibody heavy chain variable domain (VH) comprising the variable heavy chain of SEQ ID NO: 15; and
b) an antibody light chain variable domain (VL) comprising variable light chain of SEQ ID NO: 16.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the variable heavy chain of SEQ ID NO: 15; and
b) an antibody light chain variable domain (VL) comprising variable light chain of SEQ ID NO: 16.

In another preferred embodiment, said anti-CD117 agent comprises an antigen binding region comprising:
a) an antibody heavy chain variable domain (VH) comprising the variable heavy chain of SEQ ID NO: 15; and
b) an antibody light chain variable domain (VL) comprising variable light chain of SEQ ID NO: 62.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the variable heavy chain of SEQ ID NO: 15; and
b) an antibody light chain variable domain (VL) comprising variable light chain of SEQ ID NO: 62.

In another preferred embodiment, said anti-CD117 agent comprises an antigen binding region comprising:
a) an antibody heavy chain variable domain (VH) comprising the variable heavy chain of SEQ ID NO: 15; and
b) an antibody light chain variable domain (VL) comprising variable light chain of SEQ ID NO: 63.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the variable heavy chain of SEQ ID NO: 15; and
b) an antibody light chain variable domain (VL) comprising variable light chain of SEQ ID NO: 63.

In another preferred embodiment, said anti-CD117 agent comprises an antigen binding region comprising:
a) an antibody heavy chain variable domain (VH) comprising the three CDRs VHCDR1, VHCDR2 and VHCDR3 wherein VHCDR1 is SEQ ID NO: 25, VHCDR2 is SEQ ID NO: 26 and VHCDR3 is SEQ ID NO: 27; and
b) an antibody light chain variable domain (VL) comprising the three CDRs VLCDR1, VLCDR2 and VLCDR3 wherein VLCDR1 is SEQ ID NO: 28, VLCDR2 is SEQ ID NO: 29 and VLCDR3 is SEQ ID NO: 30.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 25, VHCDR2 is SEQ ID NO: 26 and VHCDR3 is SEQ ID NO: 27; and
b) an antibody light chain variable domain (VL) comprising the three CDRs VLCDR1, VLCDR2 and VLCDR3 wherein VLCDR1 is SEQ ID NO: 28, VLCDR2 is SEQ ID NO: 29 and VLCDR3 is SEQ ID NO: 30.

In another preferred embodiment, said anti-CD117 agent comprises an antigen binding region comprising:
a) an antibody heavy chain variable domain (VH) comprising the variable heavy chain of SEQ ID NO: 23; and
b) an antibody light chain variable domain (VL) comprising variable light chain of SEQ ID NO: 24.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the variable heavy chain of SEQ ID NO: 23; and
b) an antibody light chain variable domain (VL) comprising variable light chain of SEQ ID NO: 24.

In another preferred embodiment, said anti-CD117 agent comprises an antigen binding region comprising:
a) an antibody heavy chain variable domain (VH) comprising the three CDRs VHCDR1, VHCDR2 and VHCDR3 wherein VHCDR1 is SEQ ID NO: 33, VHCDR2 is SEQ ID NO: 34 and VHCDR3 is SEQ ID NO: 35; and
b) an antibody light chain variable domain (VL) comprising the three CDRs VLCDR1, VLCDR2 and VLCDR3 wherein VLCDR1 is SEQ ID NO: 36, VLCDR2 is SEQ ID NO: 37 and VLCDR3 is SEQ ID NO: 38.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 33, VHCDR2 is SEQ ID NO: 34 and VHCDR3 is SEQ ID NO: 35; and
b) an antibody light chain variable domain (VL) comprising the three CDRs VLCDR1, VLCDR2 and VLCDR3 wherein VLCDR1 is SEQ ID NO: 36, VLCDR2 is SEQ ID NO: 37 and VLCDR3 is SEQ ID NO: 38.

In another preferred embodiment, said anti-CD117 agent comprises an antigen binding region comprising:
a) an antibody heavy chain variable domain (VH) comprising the variable heavy chain of SEQ ID NO: 31; and
b) an antibody light chain variable domain (VL) comprising variable light chain of SEQ ID NO: 32.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the variable heavy chain of SEQ ID NO: 31; and
b) an antibody light chain variable domain (VL) comprising variable light chain of SEQ ID NO: 32.

In another preferred embodiment, said anti-CD117 agent comprises an antigen binding region comprising:
a) an antibody heavy chain variable domain (VH) comprising the three CDRs VHCDR1, VHCDR2 and VHCDR3 wherein VHCDR1 is SEQ ID NO: 41, VHCDR2 is SEQ ID NO: 42 and VHCDR3 is SEQ ID NO: 43; and
b) an antibody light chain variable domain (VL) comprising the three CDRs VLCDR1, VLCDR2 and VLCDR3 wherein VLCDR1 is SEQ ID NO: 44, VLCDR2 is SEQ ID NO: 45 and VLCDR3 is SEQ ID NO: 46.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 41, VHCDR2 is SEQ ID NO: 42 and VHCDR3 is SEQ ID NO: 43; and
b) an antibody light chain variable domain (VL) comprising the three CDRs VLCDR1, VLCDR2 and VLCDR3 wherein VLCDR1 is SEQ ID NO: 44, VLCDR2 is SEQ ID NO: and VLCDR3 is SEQ ID NO: 46.

In another preferred embodiment, said anti-CD117 agent comprises an antigen binding region comprising:
a) an antibody heavy chain variable domain (VH) comprising the variable heavy chain of SEQ ID NO: 39; and
b) an antibody light chain variable domain (VL) comprising variable light chain of SEQ ID NO: 40.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the variable heavy chain of SEQ ID NO: 39; and
b) an antibody light chain variable domain (VL) comprising variable light chain of SEQ ID NO: 40.

In another preferred embodiment, said anti-CD117 agent comprises an antigen binding region comprising:
a) an antibody heavy chain variable domain (VH) comprising the three CDRs VHCDR1, VHCDR2 and VHCDR3 wherein VHCDR1 is SEQ ID NO: 49, VHCDR2 is SEQ ID NO: 50 and VHCDR3 is SEQ ID NO: 51; and
b) an antibody light chain variable domain (VL) comprising the three CDRs VLCDR1, VLCDR2 and VLCDR3 wherein VLCDR1 is SEQ ID NO: 52, VLCDR2 is SEQ ID NO: 53 and VLCDR3 is SEQ ID NO: 54.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the three CDRs VHCDR1, VHCDR2 and VHCDR3 wherein VHCDR1 is SEQ ID NO: 49, VHCDR2 is SEQ ID NO: 50 and VHCDR3 is SEQ ID NO: 51; and
b) an antibody light chain variable domain (VL) comprising the three CDRs VLCDR1, VLCDR2 and VLCDR3 wherein VLCDR1 is SEQ ID NO: 52, VLCDR2 is SEQ ID NO: 53 and VLCDR3 is SEQ ID NO: 54.

In another preferred embodiment, said anti-CD117 agent comprises an antigen binding region comprising:
a) an antibody heavy chain variable domain (VH) comprising the variable heavy chain of SEQ ID NO: 47; and
b) an antibody light chain variable domain (VL) comprising variable light chain of SEQ ID NO: 48.

In another preferred embodiment, said anti-CD117 agent competes with an antigen binding region for binding to CD117 or a variant of CD117, wherein said anti-CD117 agent comprises:
a) an antibody heavy chain variable domain (VH) comprising the variable heavy chain of SEQ ID NO: 47; and
b) an antibody light chain variable domain (VL) comprising variable light chain of SEQ ID NO: 48.

It is further contemplated that the antigen-binding region of the anti-CD117 may be further screened or optimized for their binding properties as above defined. In particular, it is contemplated that said antigen binding region thereof may have 1, 2, 3, 4, 5, 6, or more alterations in the amino acid sequence of 1, 2, 3, 4, 5, or 6 CDRs of monoclonal antibodies provided herein. It is contemplated that the amino acid in position 1, 2, 3, 4, 5, 6, 7, 8, 9, or of CDR1, CDR2, CDR3, CDR4, CDR5, or CDR6 of the VJ or VDJ region of the light or heavy variable region of antigen binding region may have an insertion, deletion, or substitution with a conserved or non-conserved amino acid. Such amino acids that can either be substituted or constitute the substitution are disclosed above.

In some embodiments, the amino acid differences are conservative substitutions, i.e., substitutions of one amino acid with another having similar chemical or physical properties (size, charge or polarity), which substitution generally does not adversely affect the biochemical, biophysical and/or biological properties of the antibody. In particular, the substitution does not disrupt the interaction of the antibody with the CD117 antigen. Said conservative substitution(s) are advantageously chosen within one of the following five groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W).

In a more particular embodiment, said first antigen-binding region comprises a heavy chain variable domain comprising or consisting of any one of amino acid sequences selected from SEQ ID NO: 15, 23, 31, 39 and 47 and/or a light chain variable domain comprising or consisting of any one of amino acid sequences selected from SEQ ID NO: 16, 62, 63, 24, 32, 40 and 48.

Said first antigen binding region thereof with amino acid sequences having at least 90%, for example, at least 95%, 96%, 97%, 98%, or 99% identity to any one of the above defined amino acid sequences are also part of the present disclosure, typically first antigen binding region have at least equal or higher binding activities than said first antigen binding region consisting of heavy chain consisting of any one of amino acid sequences selected from SEQ ID NO: 15, 23, 31, 39 and 47 and/or a light chain variable domain comprising or consisting of any one of amino acid sequences selected from SEQ ID NO: 16, 62, 63, 24, 32, 40 and 48.

In a particular embodiment, said anti-CD117 agent can be a bispecific CD117 antibody, comprising at least one first binding specificity for CD117, for example, one antigen-binding region of anti-CD117 as described herein and a second binding specificity for a second target epitope or target antigen.

According to the present disclosure, said anti-CD117 agent can be an immune cell harboring an antigen receptor targeting CD117, such as a CAR targeting CD117, said antigen receptor comprising an antigen binding region as described above.

In specific embodiments, said immune cell (e.g. T cell) harboring a CAR targeting CD117 recognizes a second isoform of CD117 as expressed in a patient in need thereof, and does not recognize a first isoform of CD117. In particular said immune cell may bind specifically to an epitope including the amino acids E73, T74, V120, D121, R122, S123, Y125, K127, K193, I201, K203, S239 Y259, N260, S261, D266, Y269 and/or R271 of SEQ ID NO: 1. More preferably, said immune cell binds specifically to an epitope including the amino acids E73, V120, D121, R122, S123, K127, K193, S239, Y259 and/or S261 of SEQ ID NO: 1. Even more preferably, said immune cell binds specifically to an epitope including the amino acids E73, D121, R122, S123, S239, Y259 and/or S261 of SEQ ID NO: 1. Most preferably, said immune cell binds specifically to an epitope including amino acid E73, D121 and/or S123 of SEQ ID NO: 1.

In specific embodiments, said anti-CD117 agent can be an immune cell (e.g. T cell) harboring a CAR, said CAR comprising an antigen-binding region, e.g. scFv, comprising
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 20, VLCDR2 is SEQ ID NO: 21, VLCDR3 is SEQ ID NO: 22.

In specific embodiments, said anti-CD117 agent can be an immune cell (e.g. T cell) harboring a CAR, said CAR comprising an antigen-binding region, e.g. scFv, comprising
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60.

In specific embodiments, said anti-CD117 agent can be an immune cell (e.g. T cell) harboring a CAR, said CAR comprising an antigen-binding region, e.g. scFv, comprising
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61.

In a more particular embodiment, said anti-CD117 agent can be an immune cell (e.g. T cell) harboring a CAR comprising said first antigen-binding region e.g. scFv comprising a heavy chain variable domain comprising or consisting the amino acid sequence of SEQ ID NO: 15 and/or a light chain variable domain comprising or consisting the amino acid sequence of SEQ ID NO: 16.

In a more particular embodiment, said anti-CD117 agent can be an immune cell (e.g. T cell) harboring a CAR comprising said first antigen-binding region e.g. scFv comprising a heavy chain variable domain comprising or consisting the amino acid sequence of SEQ ID NO: 15 and/or a light chain variable domain comprising or consisting the amino acid sequence of SEQ ID NO: 62.

In a more particular embodiment, said anti-CD117 agent can be an immune cell (e.g. T cell) harboring a CAR comprising said first antigen-binding region e.g. scFv comprising a heavy chain variable domain comprising or consisting the amino acid sequence of SEQ ID NO: 15 and/or a light chain variable domain comprising or consisting the amino acid sequence of SEQ ID NO: 63.

According to the present disclosure, said anti-CD117 agent can be an immune cell harboring an antigen receptor targeting CD117, such as a CAR targeting a specific isoform of CD117, said antigen receptor comprising an antigen binding region as described above and said immune cell either not expresses CD117 or expresses an isoform of CD117 which is not recognized by said CAR.

In specific embodiments, said anti-CD117 agent can be an immune cell (e.g. T cell) harboring a CAR, said CAR targeting a specific isoform of CD117 comprising an antigen-binding region, e.g. scFv, comprising:
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22;
   and said immune cell either not expresses CD117 or expresses an isoform of CD117 which is not recognized by said CAR.

In specific embodiments, said anti-CD117 agent can be an immune cell (e.g. T cell) harboring a CAR, said CAR targeting a specific isoform of CD117 comprising an antigen-binding region, e.g. scFv, comprising:
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60;
   and said immune cell either not expresses CD117 or expresses an isoform of CD117 which is not recognized by said CAR.

In specific embodiments, said anti-CD117 agent can be an immune cell (e.g. T cell) harboring a CAR, said CAR targeting a specific isoform of CD117 comprising an antigen-binding region, e.g. scFv, comprising:
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61;
   and said immune cell either not expresses CD117 or expresses an isoform of CD117 which is not recognized by said CAR.

In a more particular embodiment, said anti-CD117 agent can be an immune cell (e.g. T cell) harboring a CAR comprising said first antigen-binding region e.g. scFv comprising a heavy chain variable domain comprising or consisting of an amino acid sequence of SEQ ID NO: 15 and a light chain variable domain comprising or consisting of the amino acid sequence of SEQ ID NO: 16, and said immune cell expresses an isoform of CD117 which is not recognized by said CAR.

In another particular embodiment, said anti-CD117 agent can be an immune cell (e.g. T cell) harboring a CAR comprising said first antigen-binding region e.g. scFv comprising a heavy chain variable domain comprising or consisting of an amino acid sequence of SEQ ID NO: 15 and a light chain variable domain comprising or consisting of the amino acid sequence of SEQ ID NO: 62, and said immune cell expresses an isoform of CD117 which is not recognized by said CAR.

In another particular embodiment, said anti-CD117 agent can be an immune cell (e.g. T cell) harboring a CAR comprising said first antigen-binding region e.g. scFv comprising a heavy chain variable domain comprising or consisting of an amino acid sequence of SEQ ID NO: 15 and a light chain variable domain comprising or consisting of the amino acid sequence of SEQ ID NO: 63, and said immune cell expresses an isoform of CD117 which is not recognized by said CAR.

In a more preferred embodiment, said anti-CD117 agent is antibody Refmab #1 as described in the examples.

In another preferred embodiment said anti-CD117 agent can be an immune cell harboring a CAR targeting a specific isoform of CD117 as described in the examples.

In particular, the disclosure also relates to depleting anti-CD117 agents as disclosed above (for example CAR cell composition or antibodies) comprising a first or a second antigen binding region for use in selectively depleting the host cells or transferred cells respectively, in a subject in need thereof.

### Cells Expressing a First Isoform of CD117

The present disclosure relates to a mammalian cell, preferably a hematopoietic cell, or a population of cells expressing a first isoform of CD117 wherein said cell or population of cells express a first isoform of CD117 comprising at least one polymorphic allele in the nucleic acid encoding said first isoform, and wherein said first isoform is not recognized by the depleting agent comprising a first antigen binding region as described herein.

Said cell or population of cells are particularly useful in medical treatment in a patient expressing a second isoform of CD117.

In a particular embodiment, said cells (e.g. hematopoietic stem cell) encoding or expressing said first isoform of CD117 not recognized by a depleting agent (e.g. hematopoietic cells) are particularly useful in medical treatment to restore normal hematopoiesis after immunotherapy, such as adoptive cell transfer in a patient expressing said second isoform, in particular wherein the treatment comprises administering a therapeutically efficient amount of said hematopoietic cells expressing said first isoform of CD117 in combination with a therapeutically efficient amount of a depleting agent targeting said second isoform of CD117. In particular, said hematopoietic cells, preferably hematopoietic stem cells are administered subsequently to said depleting agent. In another particular embodiment, said hematopoietic cells, preferably hematopoietic stem cells can be administered before or concurrently to said depleting agent

In another particular embodiment, said cells expressing said first isoform of CD117 specifically recognized by depleting agent which does not bind or binds substantially weaker second isoform of CD117 are particularly useful in medical treatment in a patient expressing said second isoform of CD117, in particular to avoid severe side-effect related to transplanted cells carrying the first isoform (safety switch), wherein the treatment comprises administering a therapeutically efficient amount of a depleting agent targeting said first isoform of CD117. In particular, said hematopoietic cells, preferably immune cells harboring a CAR are administered prior to said depleting agent.

As used herein, the term cell relates to mammalian cells, preferably human cells.

In a particular embodiment, said cells are hematopoietic cells. Hematopoietic cells comprise immune cells including lymphocytes, such as B cells and T cells, natural killer cells, myeloid cells, such as monocytes, macrophages, eosinophils, mast cells, basophils, granulocytes, dendritic cells (DC) and plasmacytoid dendritic cells (pDCs).

In a preferred embodiment, said immune cells are T cells. In another preferred embodiment, said immune cells are primary T cells. As used herein, the term “T cell” includes cells bearing a T cell receptor (TCR) or a cell derived from a T cell bearing a TCR. T-cells according to the disclosure can be selected from the group consisting of inflammatory T-lymphocytes, cytotoxic T-lymphocytes, regulatory T-lymphocytes, memory T-lymphocytes, tumor infiltrating lymphocytes or helper T-lymphocytes included both type 1 and 2 helper T cells and Th17 helper cells. In another embodiment, said cell can be derived from the group consisting of CD4+T-lymphocytes and CD8+T-lymphocytes or non-classical T cells such as MR1 restricted T cells, MAIT cells, NKT cells, gamma delta T cells or innate-like T cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person. Alternatively, T cells can be differentiated from iPS cells.

In another preferred embodiment, said hematopoietic cells are hematopoietic stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human stem cells are CD34+ cells. Hematopoietic stem cells can be differentiated from iPS cells or can be harvested from umbilical cord blood, from bone marrow or from mobilized or not mobilized peripheral blood.

In certain embodiments, the cell is an allogeneic cell which refers to a cell derived from a donor that presents with an HLA genotype that is identical, similar or different to the HLA genotype of the person receiving the cell. The donor may be a related or unrelated person. In certain embodiments, the cell is an autologous cell which refers to a cell derived from the same person that is receiving the cell.

Said cells may originate from a healthy donor or from a patient, in particular from a patient diagnosed with cancer, genetic disease or an auto-immune disease or from a patient diagnosed with an infection. Hematopoietic cells can be extracted from blood, bone marrow or derived from stem cells. HSC's can for example be derived from iPS (induced pluripotent stem cells.

A person skilled in the art will choose the more appropriate cells according to the patient or subject to be transplanted.

The disclosure further relates to a composition of cells or a population of cells for use in the therapy as disclosed herein.

### CAR

For use in adoptive cell transfer therapy, said cell expressing first isoform of CD117 according to the present disclosure may be modified to display desired specificities and enhanced functionalities. In a particular embodiment, said cell may express a recombinant antigen binding region, also named antigen receptor on its cell surface as described above. In a particular embodiment, said recombinant antigen receptor is a chimeric antigen receptor (CAR). According to the present disclosure, said immune cell expressing a first isoform of CD117 and a CAR can be specifically depleted by the administration of a therapeutically efficient amount of an agent which comprises a second antigen binding region which specifically binds to said first isoform of CD117 but not to the second isoform of CD117, thereby avoiding eventual severe side effects due to transplantation of said immune cells.

In a particular embodiment, the immune cell is redirected against a cancer antigen. By “cancer antigen” is meant any antigen (i.e., a molecule capable of inducing an immune response) which is associated with cancer. An antigen as defined herein may be any type of molecule which induces an immune response, e.g., it may be a polysaccharide or a lipid, but most preferably it is a peptide (or protein). Human cancer antigens may be human or human-derived. A cancer antigen may be a tumor-specific antigen, by which is meant an antigen which is not found in healthy cells. Tumor-specific antigens generally result from mutations, in particular frame-shift mutations which generate a wholly new amino acid sequence not found in the healthy human proteome.

Cancer antigens also include tumor-associated antigens, which are antigens whose expression or production is associated with, but not limited to, tumor cells. Examples of tumor-associated antigens include for instance Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, CD123, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-DI, muscle-specific actin, neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), CD 19, CD22, CD33, CD123, CD27, CD30, CD70, GD2 (ganglioside G2), EGFRvIII (epidermal growth factor variant III), sperm protein 17 (SpI7), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin αvβ3 (CD61), galactin, K-Ras (V-Ki-ras2 Kirsten rat sarcoma viral oncogene), or Ral-B.

In a particular embodiment, for use in adoptive cell transfer therapy, preferably for the treatment of malignant hematopoietic disease such as acute myeloid leukemia (AML) or B-acute lymphoblastic leukemia (B-ALL), the immune cell according to the present disclosure expresses a recombinant antigen binding region such as a CAR targeting CD117. Said cell expressing the first isoform and expressing the CAR (e.g. CAR-CD117) can be further specifically depleted by administering a depleting agent comprising a second antigen-binding region which binds specifically to the first isoform of CD117, but does not bind or binds substantially weaker to the second isoform of CD117, thereby avoiding eventual severe side effects such as graft-versus-host disease due to the transplantation.

In specific embodiments, said immune cell (e.g. T cell) expressing the first isoform harbors a CAR targeting CD117, said CAR comprising an antigen-binding region, e.g. scFv, comprising an antigen-binding region which binds specifically to an epitope of CD117 located within the N-terminal domain, or within the polypeptide including the amino acids E73, T74, V120, D121, R122, S123, Y125, K127, K193, I201, K203, S239 Y259, N260, S261, D266, Y269 and/or R271, more preferably amino acids E73, V120, D121, R122, S123, K127, K193, S239, Y259 and/or S261 of SEQ ID NO: 1, even more preferably amino acids E73, D121, R122, S123, S239, Y259 and/or S261 of SEQ ID NO: 1, and most preferably amino acid E73, D121 and/or S123 of SEQ ID NO: 1.

In particular, said immune cell (e.g. T cell) expressing first isoform harbors a CAR targeting CD117 comprising an antigen-binding region, e.g. scFv, comprising
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22,
   more preferably comprising an antigen-binding region comprising a heavy chain variable domain comprising or consisting of the amino acid sequence of SEQ ID NO: 15 and/or a light chain variable domain comprising or consisting of the amino acid sequence of SEQ ID NO: 16.

Alternatively, said immune cell (e.g. T cell) expressing first isoform harbors a CAR targeting CD117 comprising an antigen-binding region, e.g. scFv, comprising
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60,
   more preferably comprising an antigen-binding region comprising a heavy chain variable domain comprising or consisting of the amino acid sequence of SEQ ID NO: 15 and/or a light chain variable domain comprising or consisting of the amino acid sequence of SEQ ID NO: 62.

Alternatively, said immune cell (e.g. T cell) expressing first isoform harbors a CAR targeting CD117 comprising an antigen-binding region, e.g. scFv, comprising
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
b) an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61,
   more preferably comprising an antigen-binding region comprising a heavy chain variable domain comprising or consisting of the amino acid sequence of SEQ ID NO: 15 and/or a light chain variable domain comprising or consisting of the amino acid sequence of SEQ ID NO: 63.

### In Vitro Method for Preparing Cell Expressing First Isoform

The cell expressing the first isoform of CD117 according to the present disclosure can be genetically engineered by introducing into said cell a nucleic acid construct (e.g., mRNA) encoding at least one gene editing enzyme or ribonucleoprotein complex comprising gene editing enzyme and/or HDR template as described above. Alternatively, the gene editing system is transduced into said cells via a viral system, such as an adenoviral system. Said cell can also be genetically engineered by further introducing into said cell a nucleic acid construct encoding a CAR as described above. In particular, said method is an ex vivo method performed on a culture of cells.

The term “nucleic acid construct” as used herein refers to a nucleic acid molecule resulting from the use of recombinant DNA technology. A nucleic acid construct is a nucleic acid molecule, either single- or double-stranded, which has been modified to contain segments of nucleic acid sequences, which are combined and juxtaposed in a manner, which would not otherwise exist in nature. A nucleic acid construct usually is a “vector”, i.e., a nucleic acid molecule which is used to deliver exogenously created DNA into a host cell.

Preferably, the nucleic acid construct comprises said gene editing enzyme, HDR template and/or CAR, operably linked to one or more control sequences. Said control sequences may be a ubiquitous, tissue-specific or inducible promoter which is functional in cells of target organs (i.e., hematopoietic cell). Such sequences which are well-known in the art include in particular a promoter, and further regulatory sequences capable of further controlling the expression of a transgene, such as without limitation, enhancer, terminator, intron, silencer.

The nucleic acid construct as described above may be contained in an expression vector. The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated.

Examples of appropriate vectors include, but are not limited to, recombinant integrating or non-integrating viral vectors and vectors derived from recombinant bacteriophage DNA, plasmid DNA or cosmid DNA. Preferably, the vector is a recombinant integrating or non-integrating viral vector. Examples of recombinant viral vectors include, but not limited to, vectors derived from herpes virus, retroviruses, lentivirus, vaccinia viruses, adenoviruses, adeno-associated viruses or bovine papilloma viruses.

The present disclosure relates to a method for expressing a first isoform of a cell surface protein in a cell by introducing into said cell a nucleic acid construct (e.g. mRNA) encoding the gene editing enzyme or ribonucleoprotein complex comprising gene editing enzyme and/or HDR template as described above. Said method may further comprise a step of introducing into said cell a nucleic acid construct encoding a CAR. Said method involves introducing gene editing enzyme such as Cas protein, base editor or prime editor and guide RNA (crRNA, tracrRNa, or fusion guide RNA or pegRNA) into a cell. In particular, said gene editing enzyme is CRISPR/cas gene editing enzyme as described above. In a more particular embodiment, said gene editing enzyme is a site-specific nuclease, more preferably CRISPR/Cas nuclease comprising a guide RNA and Cas protein, wherein said guide RNA in combination with Cas protein cleaves and induces cleavage within said target sequence comprising a nucleic acid encoding surface protein region involved in agent binding as described above.

In a preferred embodiment, said nucleic acid construct comprises CRISPR/Cas nuclease capable of targeting a nucleic acid sequence encoding the CD117 region involved in binding to the depleting agent, preferably wherein said nucleic acid construct comprises:
a gRNA sequence of: SEQ ID NO: 3 to 8 which targets a sequence encoding amino acid residue E73 of SEQ ID NO: 1, or
a gRNA sequence selected from the group consisting of: SEQ ID NO: 9 to 14 which targets a sequence encoding amino acid residues V120, D121, R122, S123 of SEQ ID NO: 1.

Said Cas nuclease may be a high-fidelity Cas nuclease such as a high fidelity Cas9 nuclease.

Said gene editing enzyme, preferably guide RNA and/or Cas protein, base editor or prime editor as described above may be synthesized in situ in the cell as a result of the introduction of nucleic acid construct, preferably expression vector encoding said gene editing enzyme such as guide RNA and/or Cas protein, base editor or prime editor as described above into the cell. Alternatively, said gene editing enzyme such as guide RNA and/or Cas protein, base editor or prime editor may be produced outside the cell and then introduced thereto.

Said nucleic acid construct or expression vector can be introduced into cell by any methods known in the art and include, as non-limiting examples, stable transduction methods in which the nucleic acid construct or expression vector is integrated into the cell genome, transient transfection methods in which the nucleic acid construct or expression vector is not integrated into the genome of the cell and virus-mediated methods. For example, transient transformation methods include for example microinjection, electroporation, cell squeezing, particle bombardment or in vivo targeting approaches.

### In Vivo Editing

The cell expressing the first isoform of CD117 according to the present disclosure may also be edited in vivo. Various technologies exist that enable therapeutic in vivo gene editing, including viral vectors, lipid nanoparticles and virus-like particles (see for example Cell (2022) 185:2806-27. The molecular machinery to convert CD117 into a first isoform of CD117 which is not recognized by the depleting agent can be accomplished by any of these methods.

In certain embodiments, the present disclosure relates a pharmaceutical composition comprising molecular machinery capable of in vivo editing a gene and a depleting agent,
wherein said molecular machinery capable of in vivo editing a gene comprises all components required to introduce a point mutation of wild type CD117 in a target cell into an isoform of CD117, and
wherein said depleting agent binds to wild type CD117, but not to said isoform of CD117
for use in a medical treatment in a patient in need thereof.

Preferably said isoform of CD117 is characterized by a substitution of the aspartic acid at position 121 of wild type CD117 to a lysine. Alternatively, said said isoform of CD117 is characterized by a substitution of the serine at position 123 of wild type CD117 to a lysine.

Also preferably, said depleting agent comprises
i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
ii. an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60.

Alternatively, said depleting agent comprises
i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
ii. an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61.

Alternatively, said depleting agent comprises
i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
ii. an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22.

The term “molecular machinery capable of editing a gene” as used herein refers to an arrangement of genes and/or nucleic acids required to edit a target gene in vivo when delivered to the target site or cell via a respective vehicle. The machinery also includes respective delivery vehicles, such as viral vectors, lipid nanoparticles or virus-like particles.

### Pharmaceutical Composition and Therapeutic Use

In a further aspect, the present disclosure also provides a pharmaceutical composition comprising cells or a population of cells expressing a first isoform of CD117 as described above with one or more pharmaceutically or physiologically acceptable carriers, diluents or excipients.

In a particular embodiment, said cell expressing the first isoform of CD117 is a hematopoietic stem cell.

In another particular embodiment, said cell expressing said first isoform of CD117 is an immune cell, preferably a T-cell, more preferably a primary T cell, bearing a chimeric antigen receptor (CAR), preferably a CAR which targets the second isoform of CD117 expressed by said patient's cells as described above.

The pharmaceutical composition may further comprise a depleting agent comprising a first or second antigen binding region as described above.

The pharmaceutical composition is formulated in a pharmaceutically acceptable carrier according to the route of administration. Preferably, the composition is formulated to be administered by intravenous injection. Pharmaceutical compositions suitable for such administration may comprise the cells expressing first isoform as described above, in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents.

Optionally, the composition comprising cells expressing first isoform of CD117 may be frozen for storage at any temperature appropriate for storage of the cells. For example, the cells may be frozen at about −20° C., −80° C. or any other appropriate temperature. Cryogenically frozen cells may be stored in appropriate containers and prepared for storage to reduce risk of cell damage and maximize the likelihood that the cells will survive thawing. Alternatively, the cells may also be maintained at room temperature of refrigerated, e.g., at about 4° C.

The present disclosure relates to the cell or population of cells expressing a first isoform od CD117 as described above for use as a medicament, in particular for use in immunotherapy such as adoptive cell transfer therapy in a patient.

According to the present disclosure, said cell or population of cells (e.g., hematopoietic cells) expressing a first isoform of CD117 as described above, is used in a medical treatment in a patient in need thereof, wherein said medical treatment comprises administering a therapeutically efficient amount of cell or population of cells expressing said first isoform of CD117, in combination with a therapeutically efficient amount of a depleting agent (e.g. a CAR cell or antibody) that binds specifically to the second isoform or first isoform of CD117 to specifically depleting the patients or the transplanted cells, respectively.

As used herein, the term “in combination” or “in combination therapy” means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. The delivery can be such that an effect of the first treatment delivered is still detectable when the second is delivered. In one embodiment, a depleting agent that binds to a second isoform or a first isoform of CD117 is administered at a dose and/or dosing schedule described herein, and the cells expressing the first isoform are administered at a dose and/or a dosing schedule described herein. In some embodiments, “in combination with,” is not intended to imply that the depleting agent targeting the second (e.g. CAR cells or antibody recognizing a second isoform of CD117) or the first isoform of CD117 and compositions of cells expressing said first isoform of CD117, must be administered at the same time and/or formulated for delivery together, although these methods of delivery are within the scope of this disclosure. The depleting agent (e.g. CAR cells or antibody targeting a second isoform of CD117) can be administered concurrently with, prior to or subsequent to a dose of the hematopoietic stem cells expressing the first isoform of CD117. In certain embodiments, each agent will be administered at a dose and/or on a time schedule determined for that particular agent.

Adoptive cell transfer therapy according to the disclosure can be used to treat patients diagnosed with cancer, genetic disease, autoimmune disease, infectious disease, a disease requiring a hematopoietic stem cell transplantation (HSCT), the prevention of organ rejection, the tumor conditioning regimen, tumor maintenance treatment, minimal residual disease, the prevention of relapse.

The present disclosure also relates to the use of cells expressing a first isoform of CD117 as described above in the manufacture of a medicament for adoptive transfer cell therapy in a patient.

As used herein, the term “subject”, or “patient” refers to an animal, preferably to a mammal in which an immune response can be elicited including human, pig, chimpanzee, dog, cat, cow, mouse, rabbit or rat. More preferably, the patient is a human, including adult, child and human at the prenatal stage.

As used herein, the term “treatment”, “treat” or “treating” refers to any act intended to ameliorate the health status of patients such as therapy, prevention, prophylaxis and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

Cancers that may be treated include tumors that are not vascularized, or not yet substantially vascularized, as well as vascularized tumors. The cancers may comprise non-solid tumors (such as hematological tumors, for example, leukemias and lymphomas including relapses and treatment-related tumors e.g. secondary malignancies after use of cytotoxic therapy and hematopoietic stem cell transplantation (HSCT)) or may comprise solid tumors.

The term “autoimmune disease” as used herein is defined as a disorder that results from an autoimmune response. An autoimmune disease is the result of an inappropriate and excessive response to a self-antigen.

Infectious disease is a disease caused by pathogenic microorganism such as bacteria, viruses, parasites or fungi. In particular embodiments, infections according to the disclosure occur in immunosuppressed patients, such as patients after HSCT or patients who received a solid organ transplantation.

In a preferred embodiment, the present disclosure relates to a cell expressing first isoform of CD117 as described above for use in hematological cancer, preferably leukemia, lymphoma, myeloma or other lymphoproliferative disorders. Said leukemia can be selected from the group consisting of: acute myelogenous leukemia (AML), myelodysplastic syndrome (MDS), blastic plasmacytoid dendritic cell neoplasm (BPDCN), myeloproliferative neoplasms (MPN) including chronic myelogenous leukemia (CML), myelodysplastic syndrome/myeloproliferative neoplasm (MDS/MPN) overlap syndromes including chronic myelomonocytic leukemia (CMML), chronic lymphoid leukemia (CLL), B- and T-cell non-hodgking lymphomas, acute biphenotypic leukemia, hairy cell leukemia, interleukin-3 receptor subunit alpha positive leukemia, B-cell acute lymphoblastic leukemia (B-ALL), T-cell acute lymphoblastic leukemia (T-ALL), hodgkin lymphoma (HL), systemic mastocytosis and preferably MDS, preferably AML or BPDCN.

In a particular embodiment, said cell or population of cells (e.g., hematopoietic cells) expressing a first isoform of CD117, can be used for the treatment of solid tumor, in particular for selective depletion of myeloid cells in solid tumors in a patient, to enable immunotherapy agent such as immune checkpoint inhibitors, CAR T-cells or tumor infiltrating lymphocytes to access to tumors since myeloid cells in tumors can be immunosuppressive. In this situation said cell or population of cells (e.g., hematopoietic cells) expressing a first isoform CD117 as described above, can serve to replenish the hematopoietic system that might be affected by the treatment intended to deplete the myeloid cells in solid tumors.

In another particular embodiment, said cell or population of cells (e.g., hematopoietic cells) expressing a first isoform of CD117 as described above can be used for the treatment of autoimmune disease such as lupus, multiple sclerosis, scleroderma or systemic sclerosis.

The disclosure also relates to depleting agents (for example CAR cell composition or antibodies) comprising a first or a second antigen binding region for use in selectively depleting the host cells or transferred cells respectively, in a subject in need thereof.

### Method for Depleting Specifically Patient Cells and not Transplanted Cells

According to the present disclosure, said cell or population of cells (e.g. hematopoietic cells) expressing a first isoform of CD117 as described above, is used in a medical treatment in a patient in need thereof, wherein said medical treatment comprises administering a therapeutically efficient amount of said cells or population of cells expressing said first isoform of CD117, in combination with a therapeutically efficient amount of a depleting agent (e.g. a CAR cell or antibody) that binds specifically to a second isoform of CD117.

Indeed, during immunotherapy, immunodepleting agent, such as a CAR expressing immune cells directed to CD117, can be administered to a patient to target and kill tumoral cells. However, as tumoral surface proteins are also expressed at the surface of normal hematopoietic cells, this strategy can induce severe side effects to the patients by altering hematopoiesis. To restore hematopoiesis in the patient, hematopoietic cells can be subsequently transplanted into the patient. However, these cells need to be resistant to said agent, i.e., the depleting agent for CD117 expressing cells, in order not to be targeted by it.

Thus, alternatively, according to the present disclosure, the depleting agent comprising a first antigen binding region which binds specifically to a second isoform of CD117 can be administered to ablate specifically patient cells expressing said second isoform of CD117 and not transplanted cells expressing said first isoform of CD117. The selective depletion of patient cells, but not transplanted cells, allows to reconstitute the patient with a healthy hematopoietic system which will no longer be depleted by immunodepleting agent. Thus, according to the present therapeutic use, the patients have a functional immune system rather than go through a prolonged phase of immunodepression. The use of cells according to the present disclosure eliminates infections as a major complication of current HSC transplantation.

In another embodiment, the present disclosure relates to a method for adoptive cell transfer therapy, preferably for hematopoietic stem cell transplantation to restore normal hematopoiesis in a patient having cells expressing a second isoform of CD117 comprising:
(i) administering an effective amount of a cell (e.g. hematopoietic stem cells) expressing a first isoform of CD117 wherein said cell expressing said first isoform of CD117 comprises genomic DNA with at least one polymorphic allele, preferably single nucleotide polymorphism (SNP) allele, or a genetically engineered allele in the nucleic acid encoding said first isoform and wherein said polymorphism is not present in the genome of the patient having cells expressing said second isoform of CD117 or a pharmaceutical composition thereof; and
(ii) administering a therapeutically efficient amount of an agent comprising at least a first antigen-binding region which binds specifically to said second isoform of CD117 and does not bind or binds substantially weaker to said first isoform of CD117 to deplete specifically cells expressing said second isoform of CD117 (patient's cells).

Said cells expressing the first isoform of CD117 or pharmaceutical compositions thereof are administered to a subject in combination with (e.g., before, simultaneously or following) an agent comprising a first antigen binding region as described above.

In a preferred embodiment, the depleting agent (e.g., CAR cells or antibody targeting a second isoform of CD117 is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after) a dose of the hematopoietic stem cells expressing a first isoform of said surface protein (e.g., a first isoform of CD117).

By a “therapeutically efficient amount” or “effective amount” is intended a number of cells, in particular hematopoietic stem cells expressing the first isoform of CD117 as described above administered to a subject that is sufficient to constitute a treatment as defined above, in particular restoration of normal hematopoiesis in a patient.

The administration of the cell or pharmaceutical composition according to the present disclosure may be carried out in any convenient manner, including injection, transfusion, implantation or transplantation. The compositions described herein may be administered to a patient subcutaneously, intradermal, intratumorally, intranodally, intramedullary, intramuscularly, by intravenous or intralymphatic injection, or intraperitoneally. In another embodiment, the cells or pharmaceutical compositions of the present disclosure are preferably administered by intravenous injection. The cells or pharmaceutical compositions of the present disclosure may be injected directly into a tumor, lymph node, or site of infection.

The administration of the cells or population of cells can consist of the administration of 10⁴-10⁹ cells per kg body weight, preferably 10⁵ to 10⁷ cells/kg body weight, more preferably 2×10⁶-5×10⁶ cells per kg body weight including all integer values of cell numbers within those ranges. The dosage administrated will be dependent upon the age, health and weight of the recipient, kind of concurrent treatment, if any, frequency of treatment and the nature of the effect desired. The cells or population of cells can be administrated in one or more doses. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art.

In particular, the disclosure also relates to depleting anti-CD117 agents as disclosed above (for example CAR cell composition or antibodies) comprising a first antigen binding region for use in selectively depleting the host cells in a subject in need thereof.

### Method for Depleting Specifically Transplanted Cells and not Patient Cells (Safety Switch).

According to the present disclosure, said cell or population of cells (e.g. hematopoietic cells) expressing a first isoform of CD117 as described above, is used in a medical treatment in a patient in need thereof, wherein said medical treatment comprises administering a therapeutically efficient amount of a cell or a population of cells expressing said first isoform of CD117, in combination with a therapeutically efficient amount of a depleting agent (e.g. a CAR cell or antibody) that binds specifically to said first isoform CD117.

The cell or population of cells, preferably immune cells expressing the first isoform of CD117 of the present disclosure is particularly used in adoptive transfer cell transfer therapy into a patient. Said transplanted cell expressing said first isoform of CD117 can be further depleted in patients by administering a therapeutically efficient amount of a depleting agent comprising a second antigen binding region which specifically binds to the first isoform of CD117 particularly and does not bind or binds substantially weaker to the second isoform of CD117 expressed by patient's cells to avoid eventual severe side effects such as graft-versus-host disease due to the transplantation. In this case, said agent comprising a second antigen-binding region which binds specifically to said first isoform of CD117 (expressed by transplanted cell) is administered to deplete specifically transplanted cells and not patient cells. Selective depletion of the transplanted cells constitutes an important safety feature by providing a “safety switch”.

Graft-versus-host disease (GvHD) relates to a medical complication following the receipt of transplanted tissue from a genetically different person. Immune cells in the donated tissue (the graft) recognize the recipient (the host) as foreign. In certain embodiments, the medical condition is graft-versus-host disease caused by hematopoietic stem cell transplantation or adoptive cell transfer therapy wherein immune cells are transferred into patient.

Said side effects can also occur when transplanted cells, particularly immune cells harboring a CAR have severe side effects such as cytokine release syndrome and/or neurotoxicity. In this case, the transplanted cells expressing the first isoform of CD117 can be eliminated when said cells become malignant or cause any type of unwanted on-target or off-target damage as a safety switch.

The present disclosure relates to a method for adoptive cell transfer therapy in a patient having cells expressing a second isoform of CD117 comprising:
(i) administering an effective amount of a cell expressing a first isoform of CD117 wherein said cell expressing said first isoform of CD117 comprises genomic DNA with at least one polymorphism allele, preferably single nucleotide polymorphism (SNP) allele, or a genetically engineered allele in the nucleic acid encoding said first isoform CD117 and wherein said polymorphism is not present in the genome of the patient having cells expressing said second isoform of CD117 or a pharmaceutical composition thereof; and
(ii) administering a therapeutically efficient amount of an agent comprising at least a second antigen-binding region which binds specifically to said first isoform of CD117 and does not bind or binds substantially weaker to said second isoform of CD117 to deplete specifically cells expressing said first isoform of CD117.

Said cells expressing the first isoform of CD117 or pharmaceutical compositions thereof are administered to a subject in combination with (e.g., before, simultaneously or following) an agent comprising a second antigen binding region as described above.

In a preferred embodiment, the depleting agent (e.g. CAR cells or antibody targeting a second isoform of CD117) is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after) a dose of the hematopoietic stem cells expressing a first isoform of CD117.

The administration of the cells or pharmaceutical composition according to the present disclosure may be carried out in any convenient manner, including injection, transfusion, implantation or transplantation. The compositions described herein may be administered to a patient subcutaneously, intradermal, intratumorally, intranodally, intramedullary, intramuscularly, by intravenous or intralymphatic injection, or intraperitoneally. In another embodiment, the cells or pharmaceutical compositions of the present disclosure are preferably administered by intravenous injection. The cells or pharmaceutical compositions of the present disclosure may be injected directly into a tumor, lymph node, or site of infection.

The administration of the cells or population of cells can consist of the administration of 10⁴-10⁹ cells per kg body weight, preferably 10⁵ to 10⁷ cells/kg body weight including all integer values of cell numbers within those ranges. The dosage administrated will be dependent upon the age, health and weight of the recipient, kind of concurrent treatment, if any, frequency of treatment and the nature of the effect desired. The cells or population of cells can be administrated in one or more doses. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art.

Accordingly, in specific embodiments, the disclosure relates to a depleting agent (e.g. a CAR cell or an antibody) for use in preventing or reducing the risk of severe side effects in a patient having received a cell expressing a first isoform of CD117 as described above, wherein said patient have native cells expressing a second isoform of CD117, and wherein said depleting agent comprising at least a second antigen-binding region which binds specifically to said first isoform of CD117 and does not bind or binds substantially weaker to said second isoform of CD117.

In another aspect, the present disclosure relates to a kit for expressing a first isoform CD117 as described above into a cell, said kit comprising a gene editing enzyme, such as guide RNA in combination with a Cas protein, base editor or prime editor, nucleic acid construct, expression vector as described above or isolated cell according to the present disclosure.

In certain embodiments, the present disclosure relates to the combination of
a) a mammalian cell or a population of cells expressing an isoform of CD117, wherein said isoform of CD117 is characterized by a substitution of the aspartic acid at position 121 of wild type CD117 to a lysine, and
b) a depleting agent, comprising
   i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
   ii. an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60
for use in a medical treatment in a patient in need thereof.

In certain embodiments, the present disclosure relates to the combination of
a) a mammalian cell or a population of cells expressing an isoform of CD117, wherein said isoform of CD117 is characterized by a substitution of the serine at position 123 of wild type CD117 to a lysine, and
b) a depleting agent, comprising
   i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
   ii. an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60
for use in a medical treatment in a patient in need thereof.

In certain embodiments, the present disclosure relates to the combination of
a) a mammalian cell or a population of cells expressing an isoform of CD117, wherein said isoform of CD117 is characterized by a substitution of the aspartic acid at position 121 of wild type CD117 to a lysine, and
b) a depleting agent, comprising
   i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
   ii. an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61
for use in a medical treatment in a patient in need thereof.

In certain embodiments, the present disclosure relates to the combination of
a) a mammalian cell or a population of cells expressing an isoform of CD117, wherein said isoform of CD117 is characterized by a substitution of the serine at position 123 of wild type CD117 to a lysine, and
b) a depleting agent, comprising
   i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
   ii. an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61
for use in a medical treatment in a patient in need thereof.

In certain embodiments, the present disclosure relates to the combination of
a) a mammalian cell or a population of cells expressing an isoform of CD117, wherein said isoform of CD117 is characterized by a substitution of the aspartic acid at position 121 of wild type CD117 to a lysine, and
b) a depleting agent, comprising
   i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
   ii. an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22
   for use in a medical treatment in a patient in need thereof.

In certain embodiments, the present disclosure relates to the combination of
a) a mammalian cell or a population of cells expressing an isoform of CD117, wherein said isoform of CD117 is characterized by a substitution of the serine at position 123 of wild type CD117 to a lysine, and
b) a depleting agent, comprising
   i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
   ii. an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22
for use in a medical treatment in a patient in need thereof.

### EXAMPLES

### Example 1: Generation of Anti-CD117 Fabs and MAbs

Six different anti-CD117 antibodies were generated in Fab and MAb format based on publicly available sequence information or sources. Variable chains and CDRs (Kabat) of 5 of the antibodies (Refmab's #1-#5) are shown in Table 4. The sixth antibody (Refmab #6) is antibody 104D2, a commercially available anti-CD117 antibody from BioLegend (Cat. No. 313202; alternative supplier: Dianova (#117PE-100T)). Antibody YB5.B8 was used as expression control (Invitrogen, Cat. no. 14-1179-82).

TABLE 4

| | | |
|---|---|---|
| SEQ ID No. | Comment | Sequence |

Refmab #1

| | | |
|---|---|---|
| 15 | VH | QVQLQQPGAELVKPGASVKMSCKASGYTFTSYNMHWVKQTP |
| | | GQGLEWIGVIYSGNGDTSYNQKFKGKATLTADKSSSTAYMQINS |
| | | LTSEDSAVYYCARERDTRFGNWGQGTLVTVSA |
| 16 | VL | NIVLTQSPASLAVSLGLRATISCRASESVDIYGNSFMHWYQQKP |
| | | GQPPKLLIYLASNLESGVPARFSGSGSRTDFTLTIDPVEADDAATY |
| | | YCQQNNEDPYTFGGGTKLEIK |
| 17 | HCDR1 | SYNMH |
| 18 | HCDR2 | VIYSGNGDTSYNQKFKG |
| 19 | HCDR3 | ERDTRFGN |
| 20 | LCDR1 | RASESVDIYGNSFMH |
| 21 | LCDR2 | LASNLES |
| 22 | LCDR3 | QQNNEDPYT |

Refmab #2

| | | |
|---|---|---|
| 23 | VH | QVQLVQSGAEVKKPGSSVKVSCKASGGTFSSYAISWVRQAPGQ |
| | | GLEWMGTIGPFEGQPRYAQKFQGRVTITADESTSTAYMELSSLR |
| | | SEDTAVYYCARGGYISDFDVWGQGTLVTVSS |
| 24 | VL | DIQMTQSPSSLSASVGDRVTITCRASQSISNYLAWYQQKPGKAP |
| | | KLLIYDASSLQSGVPSRFSGSGSGTDFTLTISSLQPEDFATYYCQQ |
| | | YYYESITFGQGTKVEIKR |
| 25 | HCDR1 | SYAIS |
| 26 | HCDR2 | TIGPFEGQPRYAQKFQG |
| 27 | HCDR3 | GGYISDFDV |
| 28 | LCDR1 | RASQSISNYLA |
| 29 | LCDR2 | DASSLQS |
| 30 | LCDR3 | QQYYYESIT |

Refmab #3

| | | |
|---|---|---|
| 31 | VH | EVQLVQSGAEVKKPGESLKISCKGSGYSFTNYWIGWVRQMPGK |
| | | GLEWMAIINPRDSDTRYRPSFQGQVTISADKSISTAYLQWSSLKA |
| | | SDTAMYYCARHGRGYEGYEGAFDIWGQGTLVTVSS |
| 32 | VL | DIQMTQSPSSLSASVGDRVTITCRSSQGIRSDLGWYQQKPGKAP |
| | | KLLIYDASNLETGVPSRFSGSGSGTDFTLTISSLQPEDFATYYCQQ |
| | | ANGFPLTFGGGTKVEIK |
| 33 | HCDR1 | NYWIG |
| 34 | HCDR2 | IINPRDSDTRYRPSFQG |
| 35 | HCDR3 | HGRGYEGYEGAFDI |
| 36 | LCDR1 | RSSQGIRSDLG |
| 37 | LCDR2 | DASNLET |
| 38 | LCDR3 | QQANGFPLT |

Refmab #4

| | | |
|---|---|---|
| 39 | VH | EVQLVESGGGLVQPGGSLRLSCAASGFSISVYMMHWVRQAPG |
| | | KGLEWVASIYPYSGYTYYADSVKGRFTISADTSKNTAYLQMNSLR |
| | | AEDTAVYYCARYVYHALDYWGQGTLVTVSS |
| 40 | VL | DIQMTQSPSSLSASVGDRVTITCRASQSVSSAVAWYQQKPGKA |
| | | PKLLIYSASSLYSGVPSRFSGSRSGTDFTLTISSLQPEDFATYYCQQ |
| | | WAVHSLITFGQGTKVEIKR |
| 41 | HCDR1 | VYMMH |
| 42 | HCDR2 | SIYPYSGYTYYADSVKG |
| 43 | HCDR3 | YVYHALDY |
| 44 | LCDR1 | RASQSVSSAVA |
| 45 | LCDR2 | SASSLYS |
| 46 | LCDR3 | QQWAVHSLIT |

Refmab #5

| | | |
|---|---|---|
| 47 | VH | EVRLVESEGGLVQPRSSMKLSCTASGFTFSDYYMAWVRQVPEK |
| | | GLEWVANINYDGSSTYYLDSLKSRFIISRDNAKNILYLQMSSLKSE |
| | | DTATYYCARGDYYGTTYWYFDVWGTGTTVTVSS |
| 48 | VL | DIQMTQTTSSLSASLGDRVTISCRASQDISNYLNWYQQKPDGTV |
| | | KLLIYYTSRLQSGVPSRFSGSGSGTDYSLTISNLEQEDIATYFCQQG |
| | | KKLWSFGGGTKLEIKR |
| 49 | HCDR1 | DYYMA |
| 50 | HCDR2 | NINYDGSSTYYLDSLKS |
| 51 | HCDR3 | GDYYGTTYWYFDV |
| 52 | LCDR1 | RASQDISNYLN |
| 53 | LCDR2 | YTSRLQS |
| 54 | LCDR3 | QQGKKLWS |

Additional features of the antibodies, as well as the formats and isotypes of the full length antibodies are shown in Table 5.

TABLE 5

| Antibody | Format/isotype | Originator | Source |
|---|---|---|---|
| Refmab #1 | human IgG1 Fc | Broudy et al, Blood | US20200165337A1, |
| | silent (AEASS) | (1992) 79: 338-46 | SEQ ID NO 1 & 5 |
| | | Seven | |
| Refmab #2 | human IgG1 Fc | Novartis | US20180193475A1, |
| | silent (AEASS) | | SEQ ID NO 154 & |
| | | | 163 |
| Refmab #3 | human IgG1 Fc | Magenta | WO2019084064A2, |
| | silent (AEASS) | | SEQ ID NO 143 & |
| | | | 144 |
| Refmab #4 | human IgG1 Fc | Yale University; | WO2015050959, |
| | silent (AEASS) | Kolltan | SED ID NO 20 & 39 |
| Refmab #5 | human IgG1 Fc | Novartis | US20180193475A1, |
| | silent (AEASS) | | SEQ ID NO 9 & 18 |
| Refmab #6 | mouse IgG1 | Dianova | #117PE-100T |

### Example 2: Binding of MAbs to CD117 and Optimization of Assay Conditions

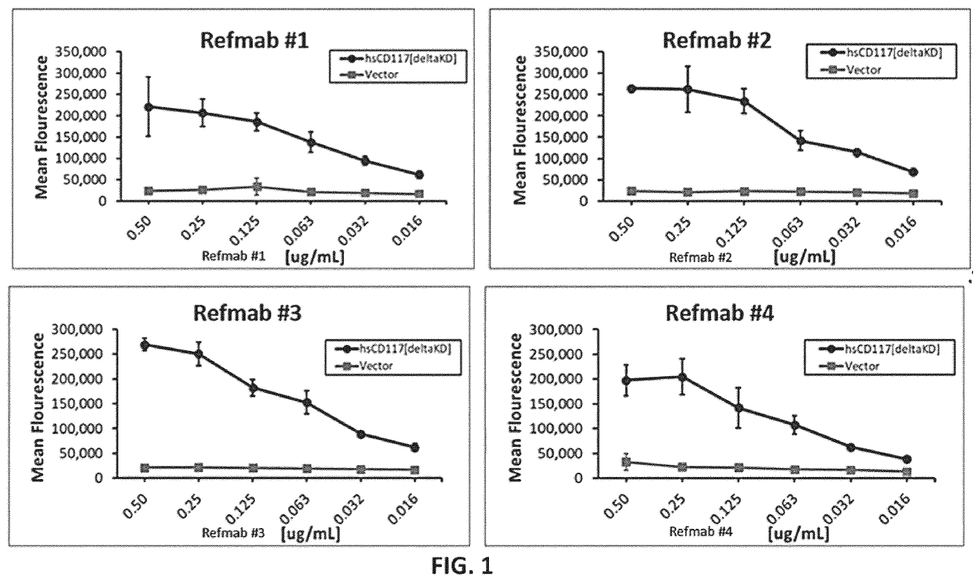

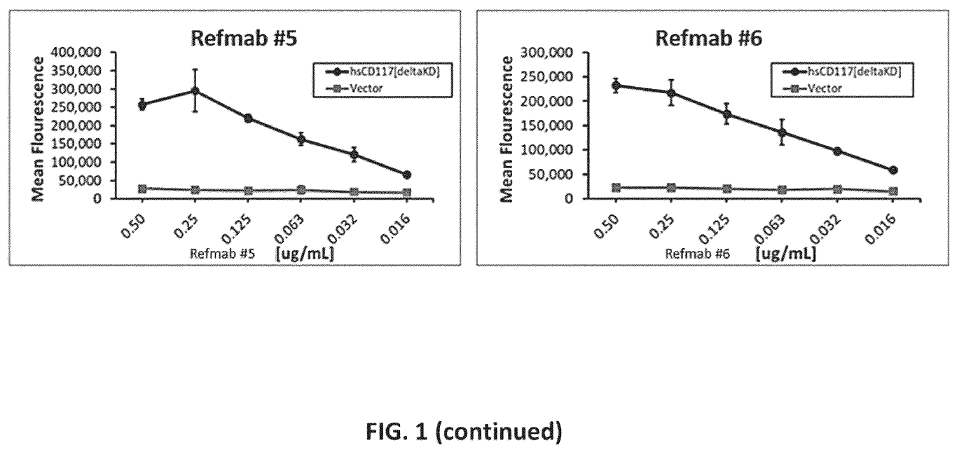

HEK-293 T cells were transfected with a construct containing wild-type CD117 (SEQ ID No. 1) or with an empty vector. Binding of the antibodies to the transfected cells and the optimal assay conditions were evaluated in 384-well format. Detection of cellular expression was measured via high-throughput flow cytometry. Serial dilutions of each antibody were tested for immunoreactivity against cells expressing CD117 or vector alone. The optimal screening concentration for each antibody was determined based on the raw signal values and signal-to-background calculations. Results are shown in FIG. 1. Each point represents the mean of four replicates.

All six antibodies in Mab format bind to human CD117 in a concentration dependent manner. Cell transfected with the empty vector did not show any binding to anti-human CD117 antibodies.

Optimized assay conditions for flow cytometry are shown in Table 6.

TABLE 6

| | Refmab #1 | Refmab #2 | Refmab #3 | Refmab #4 | Refmab #5 | Refmab #6 |
|---|---|---|---|---|---|---|
| Cell Type | HEK-293T | HEK-293T | HEK-293T | HEK-293T | HEK-293T | HEK-293T |
| Fixative | None | None | None | None | None | None |
| Blocking Buffer | 10% Goat | 10% Goat | 10% Goat | 10% Goat | 10% Goat | 10% Goat |
| | Serum | Serum | Serum | Serum | Serum | Serum |

Primary Antibody

| | | | | | | |
|---|---|---|---|---|---|---|
| Name | Refmab #1 | Refmab #2 | Refmab #3 | Refmab #4 | Refmab #5 | Refmab #6 |
| Target | CD117 | CD117 | CD117 | CD117 | CD117 | CD117 |
| Optimal Conc. | 0.125 μg/mL | 0.063 μg/mL | 0.063 μg/mL | 0.125 μg/mL | 0.125 μg/mL | 0.063 μg/mL |
| Incubation | 60 min | 60 min | 60 min | 60 min | 60 min | 60 min |
| (25° C.) | | | | | | |

Secondary Antibody

| | | | | | | |
|---|---|---|---|---|---|---|
| Target | Human IgG | Human IgG | Human IgG | Human IgG | Human IgG | Human IgG |
| Optimal Conc. | 1:400 (3.75 | 1:400 (3.75 | 1:400 (3.75 | 1:400 (3.75 | 1:400 (3.75 | 1:400 (3.75 |
| | μg/mL) | μg/mL) | μg/mL) | μg/mL) | μg/mL) | μg/mL) |
| Incubation | 30 min | 30 min | 30 min | 30 min | 30 min | 30 min |
| (25° C.) | | | | | | |
| Manufacturer | Jackson | Jackson | Jackson | Jackson | Jackson | Jackson |
| | ImmunoResearch | ImmunoResearch | ImmunoResearch | ImmunoResearch | ImmunoResearch | ImmunoResearch |
| Cat # | 109-545-003 | 109-545-003 | 109-545-003 | 109-545-003 | 109-545-003 | 109-545-003 |
| Antibody ID | AlexaFluor ® | AlexaFluor ® | Alexa Fluor ® | AlexaFluor ® | AlexaFluor ® | AlexaFluor ® |
| | 488 AffiniPure | 488 AffiniPure | 488 AffiniPure | 488 AffiniPure | 488 AffiniPure | 488 AffiniPure |
| | Goat Anti- | Goat Anti- | Goat Anti- | Goat Anti- | Goat Anti- | Goat Anti- |
| | Human IgG | Human IgG | Human IgG | Human IgG | Human IgG | Human IgG |
| | (H + L) | (H + L) | (H + L) | (H + L) | (H + L) | (H + L) |
| Wash buffer | PBS (Ca2+, | PBS (Ca2+, | PBS (Ca2+, | PBS (Ca2+, | PBS (Ca2+, | PBS (Ca2+, |
| | Mg2+ free) | Mg2+ free) | Mg2+ free) | Mg2+ free) | Mg2+ free) | Mg2+ free) |
| Signal:Background | 6:1 | 6:1 | 8:1 | 7:1 | 10:1 | 7:1 |

### Example 3: Binding of Fabs to CD117 and Optimization of Assay Conditions

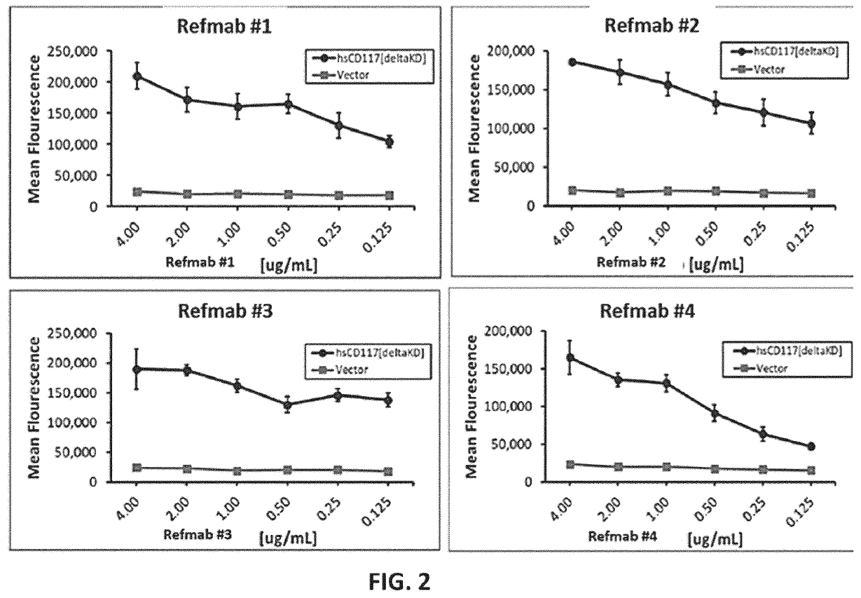

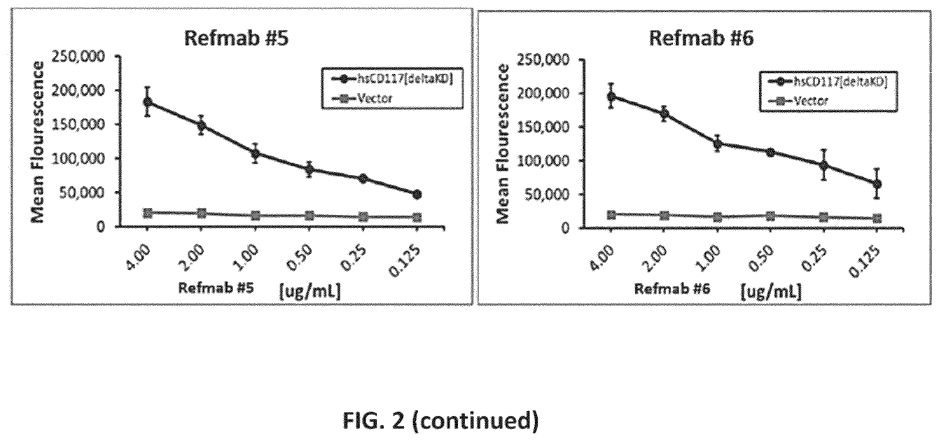

Experiments were performed similar as described in Example 2, except that Fab fragments were tested instead of full-length antibodies. Serial dilutions of each Fab were tested for immunoreactivity against cells expressing wild-type CD117 or vector alone. The optimal screening concentration for each Fab was determined based on the raw signal values and signal-to-background calculations. Results are shown in FIG. 2. Each point represents the mean of four replicates.

All six antibodies in Mab format bind to human CD117 in a concentration dependent manner. Cell transfected with the empty vector did not show any binding to anti-human CD117 antibodies.

Optimized assay conditions for high throughput flow cytometry are shown in Table 7.

TABLE 7

| Experimental | | | | | | |
|---|---|---|---|---|---|---|
| parameter | Refmab #1 | Refmab #2 | Refmab #3 | Refmab #4 | Refmab #5 | Refmab #6 |
| Cell Type | HEK-293T | HEK-293T | HEK-293T | HEK-293T | HEK-293T | HEK-293T |
| Fixative | None | None | None | None | None | None |
| Blocking Buffer | 10% Goat | 10% Goat | 10% Goat | 10% Goat | 10% Goat | 10% Goat |
| | Serum | Serum | Serum | Serum | Serum | Serum |

Primary Antibody

| | | | | | | |
|---|---|---|---|---|---|---|
| Name | Refmab #1 | Refmab #2 | Refmab #3 | Refmab #4 | Refmab #5 | Refmab #6 |
| Target | CD117 | CD117 | CD117 | CD117 | CD117 | CD117 |
| Optimal Conc. | 1.00 | 0.50 | 0.50 | 1.00 | 1.00 | 1.00 |
| (μg/mL) | | | | | | |
| Incubation | 60 min | 60 min | 60 min | 60 min | 60 min | 60 min |
| (25° C.) | | | | | | |

Secondary Antibody

| | | | | | | |
|---|---|---|---|---|---|---|
| Target | Human F(ab′)2 | Human F(ab′)2 | Human F(ab′)2 | Human F(ab′)2 | Human F(ab′)2 | Mouse F(ab′)2 |
| Optimal Conc. | 1:200 (7.50 | 1:200 (7.50 | 1:200 (7.50 | 1:200 (7.50 | 1:200 (7.50 | 1:200 (7.50 |
| | ug/mL) | ug/mL) | ug/mL) | ug/mL) | ug/mL) | ug/mL) |
| Incubation | 30 min | 30 min | 30 min | 30 min | 30 min | 30 min |
| (25° C.) | | | | | | |
| Manufacturer | Jackson | Jackson | Jackson | Jackson | Jackson | Jackson |
| | ImmunoResearch | ImmunoResearch | ImmunoResearch | ImmunoResearch | ImmunoResearch | ImmunoResearch |
| Cat # | 109-546-006 | 109-546-006 | 109-546-006 | 109-546-006 | 109-546-006 | 115-546-006 |
| Antibody ID | AlexaFluor ® | AlexaFluor ® | Alexa Fluor ® | AlexaFluor ® | AlexaFluor ® | AlexaFluor ® |
| | 488 AffiniPure | 488 AffiniPure | 488 AffiniPure | 488 AffiniPure | 488 AffiniPure | 488 AffiniPure |
| | Goat Anti- | Goat Anti- | Goat Anti- | Goat Anti- | Goat Anti- | Goat Anti- |
| | Human IgG | Human IgG | Human IgG | Human IgG | Human IgG | Mouse IgG |
| | F(ab′)2 | F(ab′)2 | F(ab′)2 | F(ab′)2 | F(ab′)2 | F(ab′)2 |
| | fragment | fragment | fragment | fragment | fragment | fragment |
| Wash buffer | PBS (Ca2+, | PBS (Ca2+, | PBS (Ca2+, | PBS (Ca2+, | PBS (Ca2+, | PBS (Ca2+, |
| | Mg2+ free) | Mg2+ free) | Mg2+ free) | Mg2+ free) | Mg2+ free) | Mg2+ free) |
| Signal: Background | 8:1 | 7:1 | 6:1 | 6:1 | 7:1 | 8:1 |

### Example 4: Alanine Scanning

An alanine scan on human CD117 was performed to the determine the residues on CD117 that are involved in binding to the six antibodies investigated. The alanine scan was performed via shotgun mutagenesis epitope mapping (Integral Molecular, Philadelphia/PA, USA) as described in Immunology (2014) 143, 13-20. Briefly, a mutation library of CD117 was created by high-throughput, site-directed mutagenesis. Each residue was individually mutated to alanine, with alanine codons mutated to serine. The mutant library was arrayed in 384-well microplates and transiently transfected into HEK293T cells. Following transfection, cells were incubated with the indicated antibodies (IgG or Fab) at concentrations pre-determined using an independent immunofluorescence titration curve on wild type CD117. Antibodies were detected using an Alexa Fluor 488-conjugated secondary antibody and mean cellular fluorescence was determined using Intellicyt iQue flow cytometry platform (Intellicyt/Sartorius). Mutated residues were identified as being critical to the antibody epitope if they did not support the reactivity of the test antibody but did support the reactivity of the reference antibody (antibody YB5.B8 (Invitrogen, Cat. no. 14-1179-82)). This counterscreen strategy facilitates the exclusion of mutants that are locally misfolded or that have an expression defect. Binding of each antibody to each mutant clone was determined in duplicates. For each point, background fluorescence was subtracted from the raw data, which were then normalized to antibody reactivity with wild type CD117.

Since library screens of very high-affinity antibodies sometimes fails to yield critical residues for antibody binding, high-affinity antibodies were converted into Fab format to weaken binding sufficiently to allow identification of critical residues for binding. For cases where Fab screens under standard conditions are still insufficient to identify critical residues for binding, high stringency conditions were implemented. These conditions include combinations of increased pH, increased salinity, increased temperature, and/or increased wash time. Antibodies that required high stringency conditions are denoted “HS”.

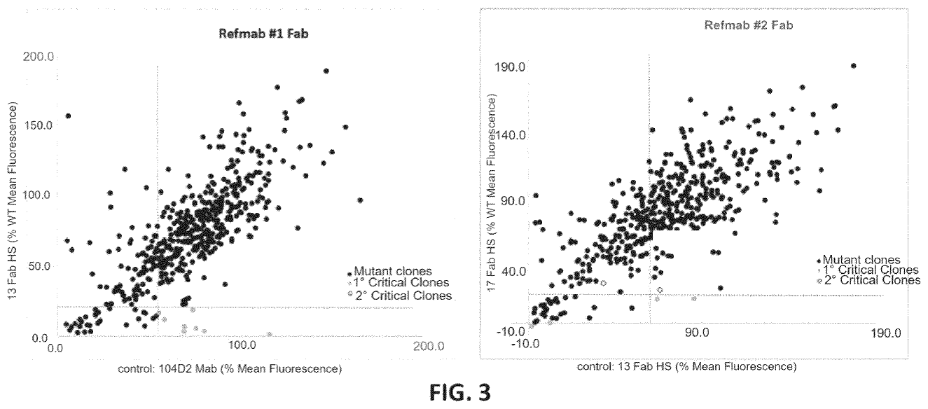

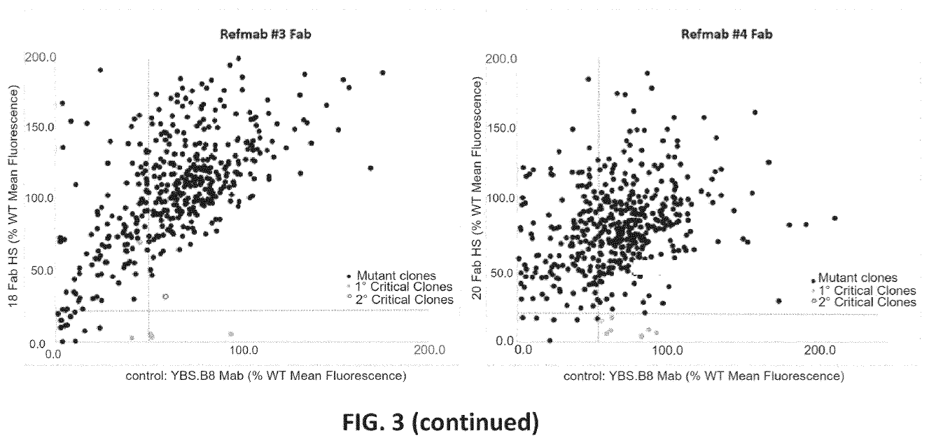

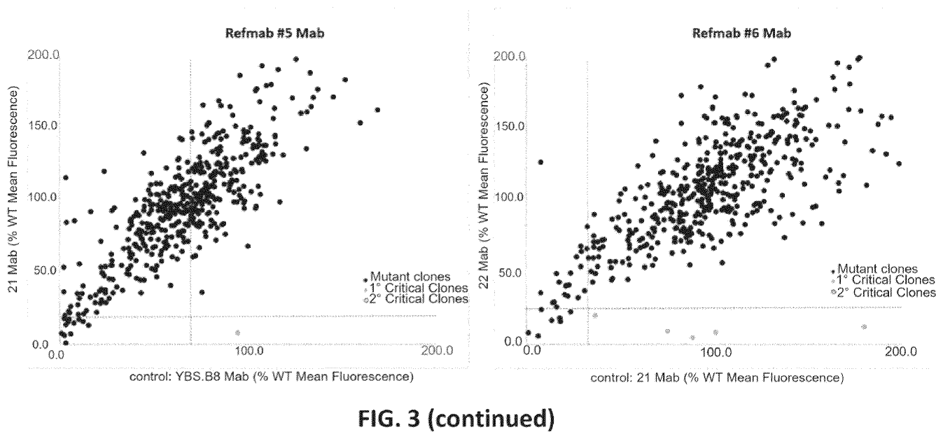

For each mutant clone, the mean binding value was plotted as a function of expression (represented by control reactivity). See FIG. 3. To identify preliminary primary critical clones (grey circles), a threshold (dashed lines) of >70% wild-type binding to control antibody and <20% wild-type binding to test antibody was applied. Secondary clones (open circles) are highlighted for clones that did not meet the set thresholds but whose decreased binding activity and proximity to critical residues suggested that the mutated residue may be part of the antibody epitope.

The key residues identified are summarized in Table 8. Mean binding reactivities (and ranges) are listed for all identified critical residues. Critical residues for antibody binding (outlined in grey) were residues whose mutations were negative for binding to test Abs, but positive for binding to control antibody. Additional secondary residues (outlined as framed cells) were identified that did not meet the threshold guidelines, but whose decreased binding activity and proximity to critical residues suggested that they may be part of the antibody epitope.

Table 9 summarizes the critical residues for each of the six antibodies tested. Residues whose mutations gave the lowest reactivities with specific antibodies are highlighted in bold and are underlined. Validated critical residues represent amino acids whose side chains make the highest energetic contributions to the antibody-epitope interaction (J. Mol. Biol. (1998) 280, 1-9; J. Mol. Biol. (1999) 285, 2177-2198); therefore, the highlighted residues are likely the major energetic contributors to binding.

TABLE 9

| Antibody | |
|---|---|
| Name | Critical Residues |
| Refmab #1 | E73, D121, R122, S123, Y125, K127, K203, S261 |
| Refmab #2 | R122, K203, S239, D266, Y269, R271 |
| Refmab #3 | K203, Y259, S261, Y269, R271 |
| Refmab #4 | M318, I319, V331, D332, Y362, H378, L379 |
| Refmab #5 | K193 |
| Refmab #6 | I39, H40, P41, R55, E81 |

### Example 5: Comprehensive Mutational Analysis

The critical residues for binding of each of the six antibodies to human CD117 identified in Example 4 were investigated in more detail. First, a validation step of identified critical residues was performed considering reproducibility of binding activity, surface accessibility, structural localization and distance to other critical sites, as well as the nature and biochemical properties of the substituted amino acid (e.g. cysteine forming disulfide bridges or post-translational modification sites). After validation, each critical residue was subject to comprehensive mutagenesis to selected biophysically appropriate non-alanine amino acids, based on sequence and structure-related properties of the substituted amino acid as well as the newly introduced ones

Antibodies were screened for binding to the human CD117 variants in IgG format. As in Example 4, binding of each test antibody to each mutant clone in the comprehensive library was determined in duplicate by high-throughput flow cytometry. For each mutation, background fluorescence was subtracted from the raw data, which were then normalized to antibody reactivity with wild-type CD117. Mean binding reactivities and ranges are listed in Table 10 for all mutant clones. Mutations that caused binding below 25% are highlighted grey.

Based on these results, and also taking into account the location of the individual residues in the three-dimensional structure, as well as their substitution propensity as estimated based on evolutionary analysis of related protein sequences, the residues shown in Table 11 were elected as most promising candidates for follow up studies.

TABLE 11

| | | Antibodies that bind |
|---|---|---|
| Residue | Mutation | to this residue |
| E73 | K, L, Q, A, G, Y, R | Refmab #1 |
| V120 | I, P | Refmab #1 |
| D121 | Y, H, K, R, T, S, V | Refmab #1 |
| R122 | S, L | Refmab #1, Refmab #2 |
| S123 | P, F, K, V | Refmab #1 |
| K127 | L | Refmab #1 |
| K193 | G, T, M, D, E | Refmab #5 |
| Y259 | E, A, P, G | Refmab #3 |
| S261 | V | Refmab #3 |

### Example 6: Biophysical Characterization of Elected CD117 Variants

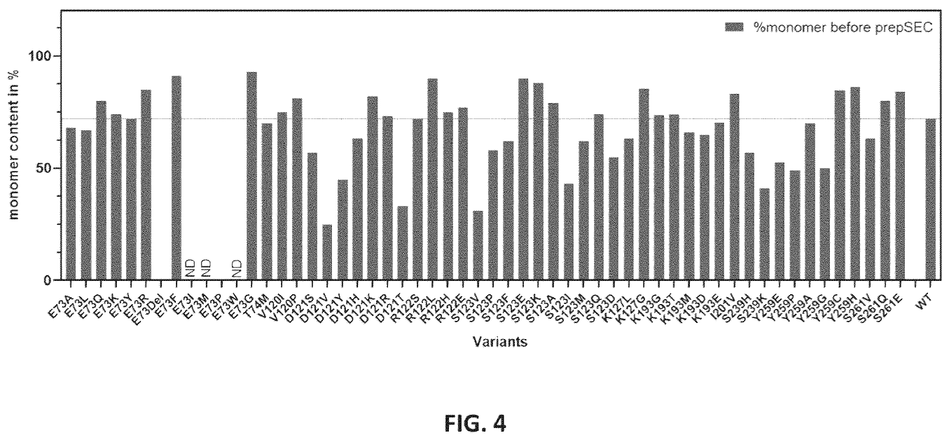

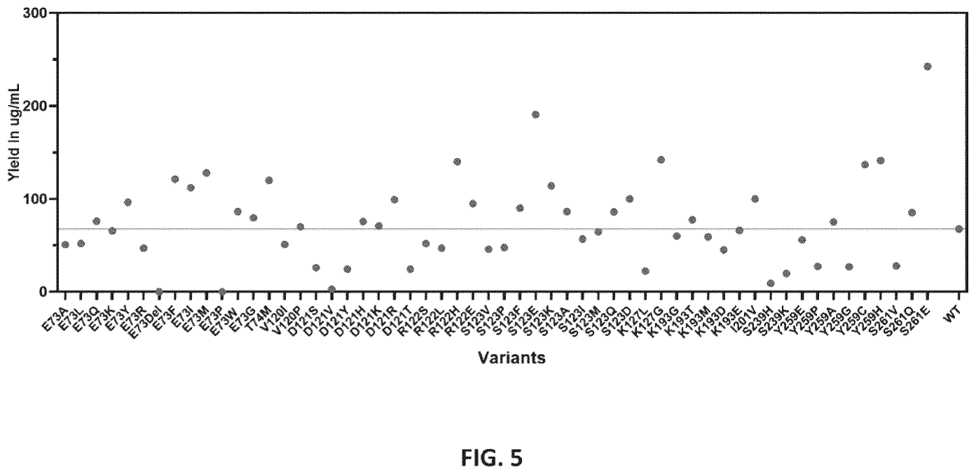

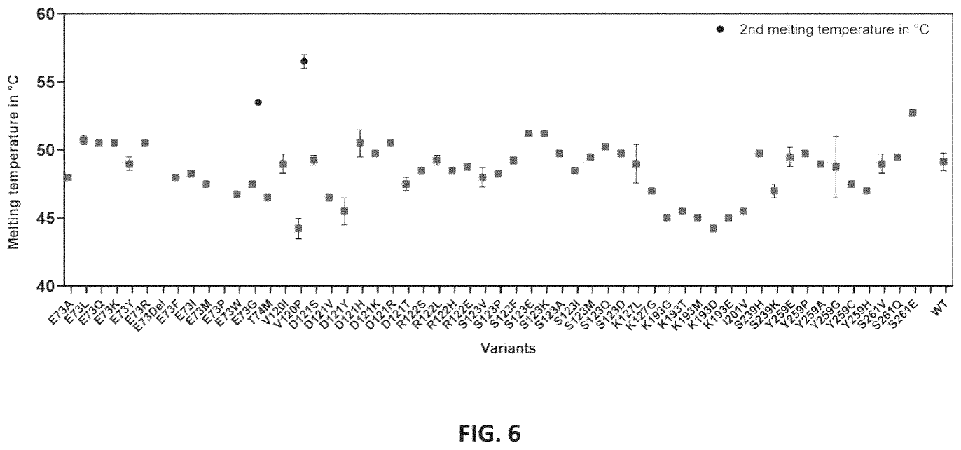

Biophysical properties of the elected variants were tested. Aggregation of the elected CD117 variants was determined by preparative size exclusion chromatography. Yield of the elected CD117 variants was determined by dividing the amount of protein after purification by the production volume. Melting temperature was measured by differential scanning fluorimetry using Sypro Orange (Sigma, PN: S5692-50 ul) and an RT-PCR machine (C1000 Thermal Cycler, Biorad). The samples were measured at 0.25-1.0 mg/ml in PBS buffer in duplicates and Sypro Orange was added at a final concentration of 5×. The temperature was sequentially increased (0.5° C. each 10 s) from 25 to 95° C. The fluorescent increase was monitored as a function of temperature and the melting temperature was determined as the inflexion point of the sigmoidal curve and compared to wild-type CD117. Results are shown in FIGS. 4 (aggregation), 5 (yield) and 6 (melting temperature).

The monomeric content of most variants was acceptable, only a few variants (D121V, D121Y, D121T, S123V, S1231, S239K) showed a monomeric content of less than 50%. Also the production yield for all but a very few variants (E73del, D121V, S239H) was sufficiently high. Likewise, melting temperatures were in an acceptable range, except for variant V120P, for which two melting points were measured, indicating a structural difference compared to wild-type CD117.

### Example 7: Binding of Antibody Refmab #1 to the Elected Variants

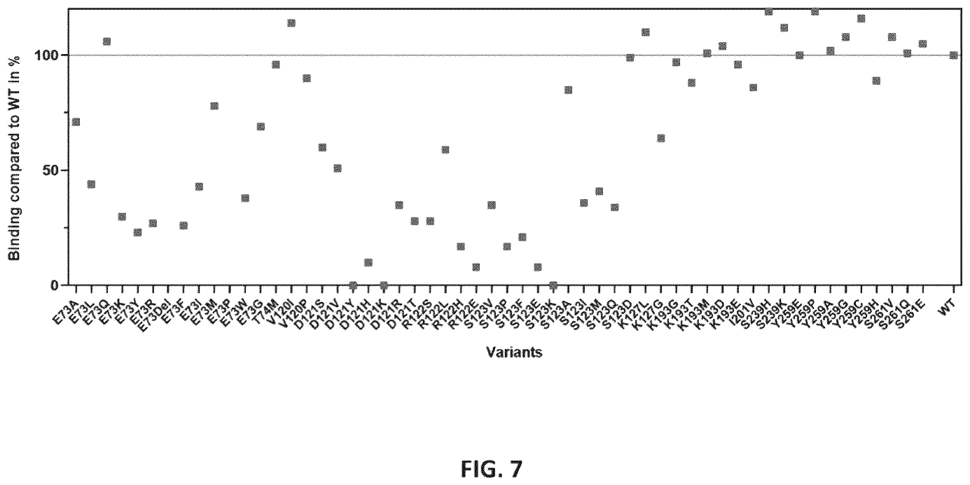

A more sophisticated analysis of binding to the elected variants was performed with antibody Refmab #1. Binding of Refmab #1 to wt and variants (analyte) was measured in an Octet system RED96e or R8 at 25° C. with shaking at 1,000 rpm using 1× kinetic buffer (Sartorius, PN: 18-1105). First, the elected variants were screened for their ability to bind Refmab #1 using three different concentrations of analytes. Antibody Refmab #1 was captured by Anti-Human Fc capture biosensor (AHC) (Sartorius, PN: 18-5060) for 300 s at 0.5 to 1 μg/mL. As an analyte, human CD117 wt and variants, containing only domains 1, 2 and 3 (CD117 D1-2-3), was titrated at 500 nM, 50 nM and 5 nM. Association and dissociation of the analyte to Refmab #1 was monitored for 300 s and 900 s, respectively. Reference subtraction was performed against buffer only wells. AHC tips were regenerated using 10 mM Gly-HCl pH 1.7. Data were analyzed using the Octet Data Analysis software HT 12.0. Data were fitted (when possible) to a 1:1 binding model. Kinetic rates ka and ka were globally fitted. Binding level of the elected variants were compared to wild type and are shown as percent in FIG. 7. For this qualitative analysis, the binding level of the top analyte concentration at end of the association step was used.

Results are shown in FIG. 7. Several CD117 variants showed a particular strong decrease in binding to Refmab #1, including E73Y, E73R, D121Y, D121H, D121K, D121T, R122S, R122H, R122E, S123P, S123F, S123E and S123K. Variants E73K, D121R, S123V, S1231, S123M and S123Q also showed a strong decrease in binding to Refmab #1. The result in the Octet system also correlated well with the result observed in FACS experiments.

To further characterize binding of Refmab #1 to CD117 D1-2-3 variants E73K, E73L, E73R, E73Y, D121H, D121K, S123K and S123F, the analytes were titrated at 7 different concentrations (from 2000 nM to 5 nM). Antibody Refmab #1 was captured by AHC biosensors for 300 s at 0.5 to 1 μg/mL. Association and dissociation to Refmab #1 was extended to 600 s and 1000 s, respectively. Steady state analysis was performed.

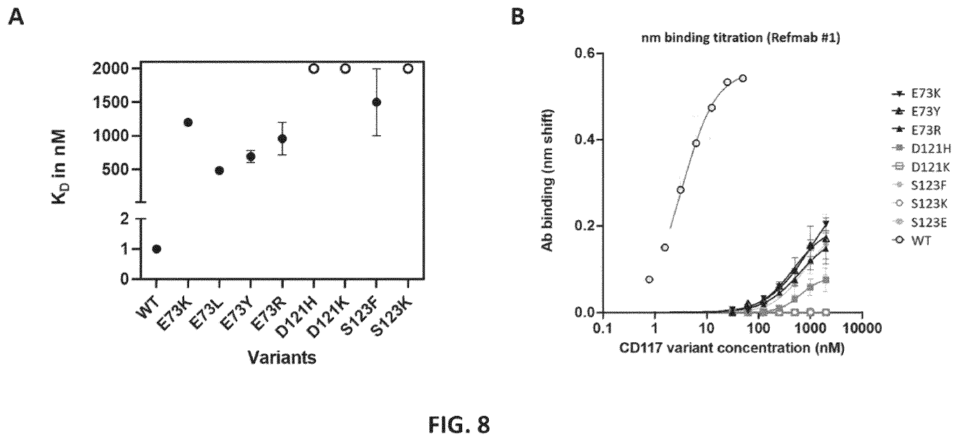

The KD of antibody Refmab #1 to the E73K variant of human CD117 decreased 1200-fold compared to wild type CD117 (1200 nM vs 1 nM, as determined by two individual experiments). The KD of antibody Refmab #1 to the E73L variant of human CD117 decreased around 500-fold compared to wild type CD117 (485 nM vs 1 nM, as determined by two individual experiments), to the E73Y variant of human CD117 around 700-fold compared to wild type CD117 (691 nM vs 1 nM, as determined by two individual experiments), to the E73R variant of human CD117 around 1000-fold compared to wild type CD117 (958 nM vs 1 nM, as determined by two individual experiments), and to the S123F variant of human CD117 around 1,500-fold compared to wild type CD117 (1500 nM vs 1 nM, as determined by two individual experiments). No binding (nm shift higher than 0.1 nm) of Refmab #1 was observed at 2000 nM of human CD117 variants D121H, D121K and S123K. See FIG. 8.

### Example 8: SCF Binding to Elected CD117 Variants

SCF binding is one function of CD117. SCF binding is important for the transmission of functional signals by CD117. Binding of the elected variants to SCF was measured on an Octet system RED96e or R8 at 25° C. with shaking at 1000 rpm using 1× Kinetic Buffer (Sartorius, PN: 18-1105). Biotinylated Avitag-hSCF (Acros Biosystems, PN: SCF-H82E1) was captured on Streptavidin (SA) biosensor (Sartorius, PN: 18-5019) for 600 s at 0.5-1 ug/mL A construct carrying domains 1, 2 and 3 of wild type CD117 (CD117 D1-2-3 wt) and a construct carrying domains 4 and 5 of wild type CD117 (CD117 D4-5 wt) were used in this experiment. Domain 1, 2, 3 comprise the binding site for SCF. Domains 4 and 5 were used as a negative, non-binding control.

CD117 D1-2-3 wt and variants thereof are titrated with different concentrations from 1000 to 5 nM. Association was monitored for 300 s and dissociation for 600 s. Binding to human CD117 domain 4 and 5 (CD117 D4-5) was performed under the same conditions as non-binding control. Reference subtraction was performed against buffer only wells. Biosensors were not regenerated and a new set of SA biosensors was used for each analyte. Data were analyzed using the Octet Data Analysis software HT 12.0. Steady state data analysis was performed due to the fast on/off nature of the interaction.

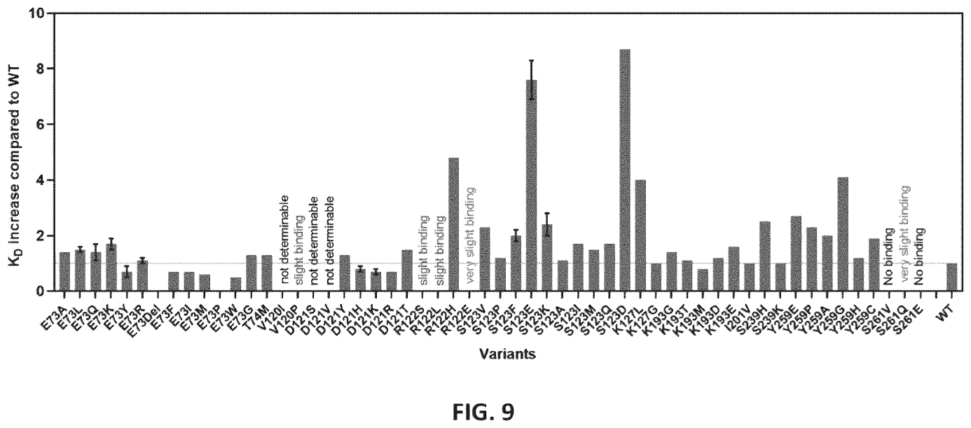

Results are shown in FIG. 9.

The elected variants showed different degrees of binding to SCF. In particular, the E73 variants (E73L, E73Q, E73K, E73Y, E73R and E73A) showed only little loss of binding to SCF, with all variants exhibiting a Kp increase of less than 2 compared to wild type CD117. This was also observed for the D121 variants D121Y, D121H, D121K, D121R and D121T, S123 variants S123P, S123F, S123K, S123A, S1231, S123M and S123Q, S239 variant S239K, and for Y259 variant Y259A.

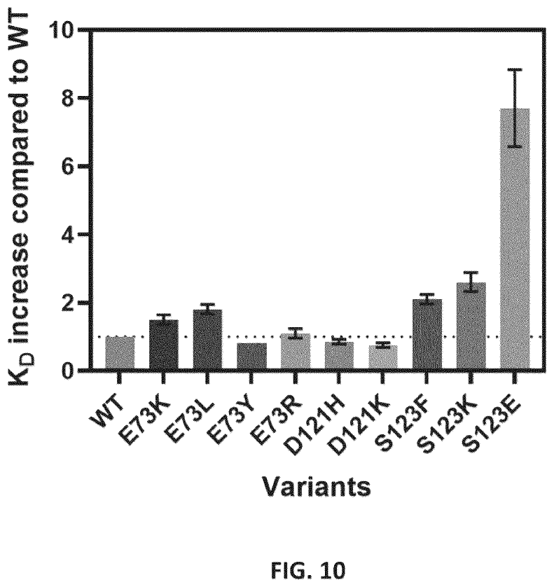

Also variants in S123V, K127L and Y259E showed binding to SCF, albeit to a slightly lower degree. The KD for the variants E73L, E73Q, E73K, E73Y, E73R, E73A, D121Y, D121H, D121K, D121R, D121T, S123P, S123F, S123A, S1231, S123M, S123Q, S239K and Y259A increased less than two-fold as compared to wild type CD117. The Kp for the variants E73A, E73L, E73Q, E73K, E73Y, E73R, D121Y, D121H, D121K, D121R, D121T, S123V, S123P, S123F, S123K, S123A, S1231, S123M, S123Q, S123V, S239H, S239K, Y259E, Y259P, and Y259A increased less than three-fold as compared to wild type CD117. The Kp for the variant E73A, E73L, E73Q, E73K, E73Y, E73R, D121Y, D121H, D121K, D121R, D121T, S123V, S123P, S123F, S123K, S123A, S1231, S123M, S123Q, S239H, S239K, Y259E, Y259P, and Y259A increased less than four-fold as compared to wild type CD117. An exemplary result is shown in FIG. 10.

A cell-based assay to measure binding of SCF was set up as well. A 1:1 mixture of biotinylated SCF and streptavidin PE was incubated for 45 min. During the incubation, TF-1 wild-type cells are seeded at 1 million cells/ml. Fc blocking was performed for 15 min (Trustain Biolegend), then 0.1-100 μg/ml of either Ref001 (an isotype control antibody with specificity for hen egg white lysozyme) or anti-CD117 antibodies are added to the cells. After additional 15 min and wash steps, a mix of biotinylated SCF and streptavidin PE is added to the cells for 30 min. Finally, 2 more wash steps are performed and a 7-AAD viability staining solution is added to the cells for 10 min. The plate is then measured via the Novocyte Quanteon flow cytometer.

### Example 9: SCF-Dependent Proliferation of the Elected CD117 Variants

SCF-dependent proliferation is another function of CD117. SCF-dependent proliferation was measured as follows: TF-1 cells were seeded at a cell concentration of 150 000 cells/ml in sterile, white cell culture treated plates in RPMI Media+10% FCS. Cells were treated with 100 ng/ml of SCF and a titration of various CD117 antibodies or an isotype control antibody at different concentrations. After 3 days of incubation a CellTiter-Glo 2.0 assay (Promega) was performed and luminescence was read in a luminometer (e.g. Envision or Phera Star plate readers).

CD117 wild-type TF-1 cells were compared to TF1 cells with a E73K knock in. E73K knock in clonal populations differ in that KI1 was originally treated with Alt-R HDR Enhancer V2 (IDT), with 61% of the alleles having been repaired via HDR-mediated editing as observed via NGS, whereas KI2 was untreated and resulted in 39% HDR. Both KI1 and KI2 were obtained upon sorting the edited TF1 cells using the SONY MA900, removing any wild-type and knock out cells from the final subsets, thus yielding high purity of knock in.

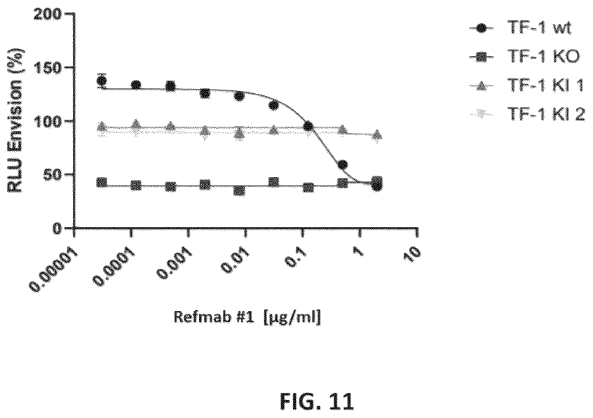

Results are shown in FIG. 11. Both, wild-type and E73K knock-in cells, strongly proliferated, whereas knock-out cells did not. Treatment with increasing concentrations of Refmab #1 led to a decrease in proliferation of wild type cells, but not in E73K cells. Cells treated with avapritinib likewise showed no proliferation for any type of cells (wild type, E73 and knock-out; data not shown).

In another experiment, SCF-dependent proliferation of variants E73K, E73Y, D121K, S123F and S123K was compared against wildtype CD117. TF-1 cells were seeded at a cell concentration of 150 000 cells/ml in sterile, white cell culture treated plates with transparent bottom in RPMI+GlutaMax+10% FCS. Cells were treated with a titration of SCF. After 3 days of incubation a CellTiter-Glo 2.0 assay (Promega) was performed, luminescence was read in a luminometer (Envision plate reader) and EC50's was determined. Two different experiments with triplicates in each experiment were performed.

The EC50 was similar for all constructs tested.

TABLE 12

| EC50 [ng/ml] | Wildtype | D121K | E73K | E73Y | S123F | S123K |
|---|---|---|---|---|---|---|
| Experiment | 4.14 | 4.15 | 3.73 | 4.37 | 4.01 | 3.76 |
| #1 | | | | | | |
| Experiment | 4.22 | 3.25 | 3.42 | 3.64 | 3.63 | 3.31 |
| #2 | | | | | | |
| Average | 4.18 ± 0.04 | 3.70 ± 0.45 | 3,58 ± 0.16 | 4.01 ± 0.37 | 3.82 ± 0.19 | 3.54 ± 0.23 |

Binding of the variants to SCF was hence not affected as compared to the wild type.

Next, proliferation of the variants was tested with increasing concentrations of RefMab #1 at a constant concentration of SCF (100 ng/ml). TF-1 cells were seeded at a cell concentration of 150 000 cells/ml in sterile, white cell culture treated plates with transparent bottom in RPMI+GlutaMax+10% FCS and a fix concentration of 100 ng/ml SCF in the media, Cells were treated with different concentrations of RefMab #1. After 3 days of incubation a CellTiter-Glo 2,0 assay (Promega) was performed, luminescence was read in a luminometer (Envision plate reader).

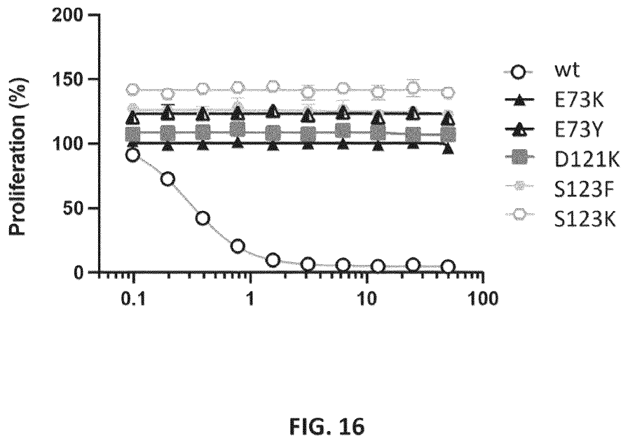

Results are show in FIG. 16. RefMab #1 had a pronounced effect on the proliferation of wild type TF-1 cells, whereas proliferation of all CD117 variants tested was not affected by RefMab #1.

For human CD34+ HSCs (different donors) cells are seeded in X-Vivo 20 medium with 100 ng/ml SCF, 50 ng/ml TPO and 50 ng/ml FLT-3 Ligand in sterile white cell culture treated plates at a cell density of 30 000 cells/ml. HSCs are then treated with various CD117 antibodies or an isotype control antibody at different concentrations and plates are incubated for 5 days. After 5 days a CellTiter-Glo 2.0 assay (Promega) is performed and luminescence is read in a luminometer (e.g. Envision or Phera Star plate readers).

### Example 10: SCF-Dependent Phosphorylation of the Elected CD117 Variants

SCF-dependent phosphorylation is another function of CD117. SCF-dependent phosphorylation was measured using TF-1 cells expressing CD117 (as shown in previous experiments). TF-1 cells were seeded at a concentration of 1 million cells/ml in 1 ml of Medium (RPMI1640 supplemented with GlutaMAX+10% heat inactivated FBS) in a 6-well plate. Cells were treated with 0.5 ug/mL antibody or 500 nM avapratinib. Cells were then treated with 100 ng/ml recombinant human SCF (1 mg/ml PeproGMP® Recombinant Human SCF) for 5 minutes, before cell collection. Cells were washed with ice-cold PBS, before resuspending cells in 250 μL Lysis Buffer (Lysis buffer from Signaling containing Protease Inhibitor and Phosphatase Inhibitor). Cells were flash frozen in liquid nitrogen and thawed at 37° C. twice, before freezing them at −80° C. until further usage. Undiluted samples were thawed and processed according to the manufacturers protocol for the PathScan® Phospho-c-Kit (Tyr719) Sandwich ELISA Kit (Cell Signaling). Absorbance (OD_450 nm) was assessed using the Envision (Perkin Elmer).

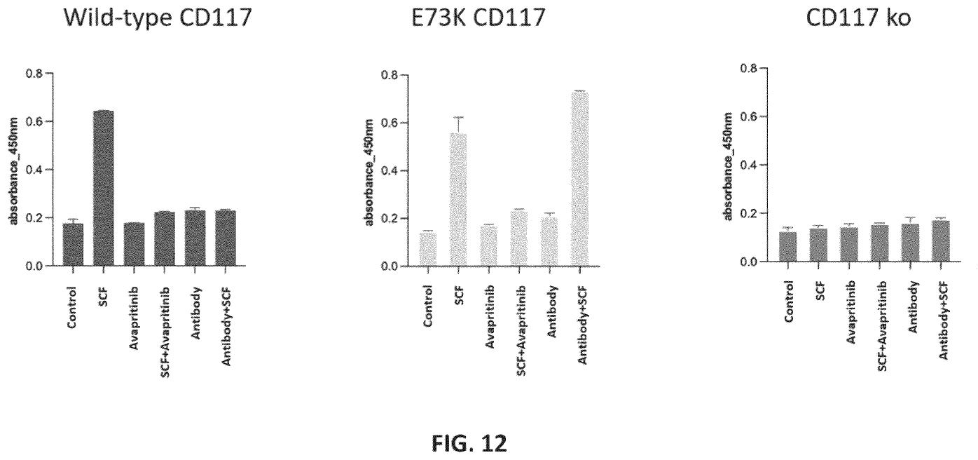

Exemplary results are shown in FIG. 12 for the E73K variant of CD117. In wild type cells, SCF-induced phosphorylation can be blocked with an antibody competing with SCF for binding to CD117. SCF-induced phosphorylation is also blocked by avapritinib, a known inhibitor of the CD117-SCF interaction. In contrast, SCF-induced phosphorylation is not blocked with the antibody for the E73K variant of CD117. Avapritinib still blocks SCF-induced phosphorylation in the E73K variant.

### Example 11: Selective Blockade/Depletion of Cells Expressing the First or the Seconds Isoform

Selected CD117 variants are introduced into TF-1 cells by genome editing. Edited cells are then incubated with a toxin coupled to SR-1. Selective depletion of cells expressing a binding (wildtype) CD117 isoform can be monitored by FACS or viability assays. The assay can be performed with bulk edited cells. In the case of HDR this will result in a mixed population of cells containing wildtype cells, KO cells and knock-in cells carrying the correctly edited variants. The three populations can be discriminated by FACS using SR-1 and 104D2. Alternatively, the 3 populations can be purified by FACS and subsequently incubated with toxin coupled to SR-1.

Cells expressing the wildtype CD117 will die while gene edited cells expressing the variants will survive.

Alternatively to toxin coupled to SR-1 other modes of action can be used including but not limited to ADCC or CAR T.

In lieu of TF-1 cells other cell lines that do not express CD117 endogenously (e.g HEK293, DF-1) can be used. Recombinant expression of wildtype or variant CD117 either transiently or stably can then be used as target cells for killing assays. Alternatively, primary human HSCs can be isolated as CD34+ cells. Gene edited CD34+ HSCs can be transferred into immunodeficient mice (e.g. NSG, NSG-SGM3 or NBSGW) for engraftment studies. This allows to monitor engraftment and differentiation of progenitor cells in vivo. In addition, a depleting agent, e.g. toxin coupled SR-1 can be applied in vivo to monitor selective depletion of binding CD117 isoforms but not non-binding CD117 variants.

### Example 12: Binding of SR-1 to Gene Edited Variants of CD117

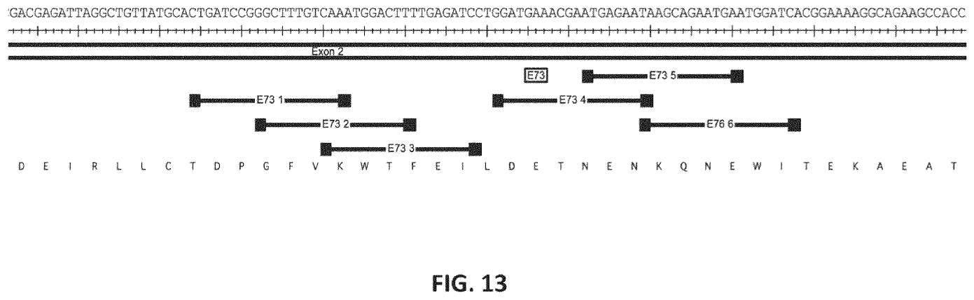

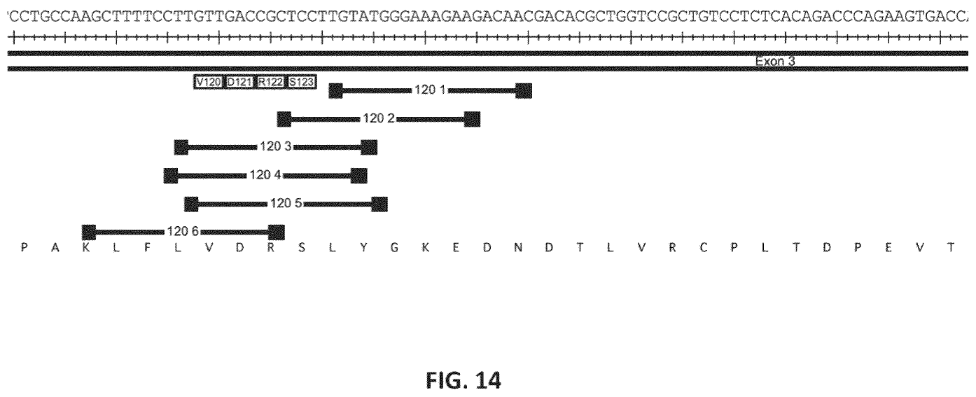

Gene editing of CD117 was performed in TF-1 cells using HDR. 12 different crRNAs were tested, six of them targeting amino acid residue E73 (Table 3), and six of them targeting amino acid residues 120-123 (Table 3). FIG. 13 shows a map depicting binding of the tested crRNAs relative to position E73. FIG. 14 shows a map depicting binding of the tested crRNAs relative to positions 120-123.

Binding of antibody SR-1 to TF-1 cells was measured by FACS 7 days after electroporation with Cas9 protein, crRNA/tracrRNA or sgRNA and HDR template (Table 4). Loss of binding to the gene edited variants as compared to wild type cells is shown in Table 13:

| | KnockOut |
|---|---|
| | Efficiency |
| gRNA | [for results] |
| KIT_E73_1 | 10.8% |
| KIT_E73_2 | 25.7% |
| KIT_E73_3 | 37.2% |
| KIT_E73_4 | 60.1% |
| KIT_E73_5 | 68.5% |
| KIT_E73_6 | 60.5% |
| KIT_120_123_1 | 64.1% |
| KIT_120_123_2 | 63.1% |
| KIT_120_123_3 | 71.2% |
| KIT_120_123_4 | 60.0% |
| KIT_120_123_5 | 53.8% |
| KIT_120_123_6 | 60.6% |

Similar results were obtained with anti-CD117 antibody 104D2.

HDR templates of SEQ ID NOs 15-18 are ordered as Alt-R™ HDR Donor Oligos (Integrated DNA Technologies). The HDR templates have 2 different lengths and are on the plus or the minus strand and the E to K mutation is introduced by at least one base mutation. HDR templates are used together with crRNA KIT_E73_4 (SEQ ID NO. 6.) HDR templates may contain at least one silent mutation in the PAM to avoid recutting.

### Example 13: Internalization of Antibodies into CD117 Variants

Antibody internalization can be tested by the skilled person by any commonly used assay, such as FACS. Cells expressing CD117 are incubated with antibodies labelled with a fluorophore e.g. Alexa Fluor 488 (AF488) for different time points (0.5-6h), before washing and quenching with an anti-AF488 antibody for one hour. Internalized antibody is able to give a signal in the FACS readout, while the signal of Antibody bound to the cell surface is quenched and not detectable.

Antibody internalization into TF-1 cells expressing CD117 or variants thereof is measured by FACS. TF-1 cells are seeded at a concentration of 1 million cells/mL in 0.1 ml of medium (RPMI1640 supplemented with GlutaMAX+10% heat inactivated FBS+2 ng/ml GM-CSF) in a 96-well plate. The next day, cells are treated with 2-20 μg/mL Antibody labelled with Alexa Fluor 488 (AF488; Alexa Fluor® 488 Conjugation Kit (Fast)-Lightning-Link®, Abcam) for 30-360 minutes at 37° C. or 4° C., before cell collection and washing in ice cold PBS. Cells are resuspended in ice cold PBS containing 20-200 ug/mL Anti-AF488 antibody (Alexa Fluor 488 Polyclonal Antibody, ThermoFisher Scientific) for one hour before acquiring data on the FACS machine (NovoCyte, Agilent). Signal of internalized antibody is measured by the FACS, while signal of antibody outside of the cell is quenched. Percentage of antibody internalization is calculated by dividing signal of cells incubated with quencher by the signal of cells incubated without quencher.

### Example 14: Binding of Antibodies Refmab's #2 and #3 to the Elected Variants

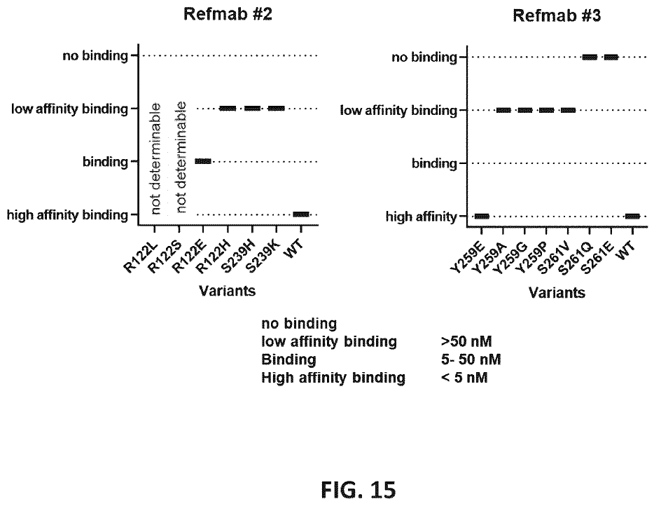

A similar experiment to that in Example 7 was performed, but utilizing Refmab's #2 and #3. Results are shown in FIG. 15.

Refmab #2 showed a strong decrease in binding to variants R122H, S239H and S239K as compared to wild type CD117. Refmab #3 showed a strong decrease in binding to variants Y259A, Y259G, Y259P and S261V as compared to wild type CD117. A complete loss of binding was observed for Refmab #3 with variants S261Q and S261E.

### Example 15: Improved Refmab Antibodies

Improved versions of antibody Refmab #1 were generated. Amino acid sequences of the improved binders, Refmab #1-Ernie and Refmab #1-Bert, are shown in Table 14.

TABLE 14

| | | |
|---|---|---|
| SEQ ID No. | Comment | Sequence |

Refmab #1-Ernie

| | | |
|---|---|---|
| 15 | VH | QVQLQQPGAELVKPGASVKMSCKASGYTFTSYNMHWVKQTP |
| | | GQGLEWIGVIYSGNGDTSYNQKFKGKATLTADKSSSTAYMQINS |
| | | LTSEDSAVYYCARERDTRFGNWGQGTLVTVSA |
| 62 | VL | EIVLTQSPSSLSASVGDRVTITCRASESVDIYGESFMHWYQQKPG |
| | | KAPKLLIYLASNLESGVPSRFSGSGSGTDFTLTISSLQPEDFATYYC |
| | | QQNNEEPYTFGQGTKLEIKRTV |
| 17 | HCDR1 | SYNMH |
| 18 | HCDR2 | VIYSGNGDTSYNQKFKG |
| 19 | HCDR3 | ERDTRFGN |
| 59 | LCDR1 | RASESVDIYGESFMH |
| 21 | LCDR2 | LASNLES |
| 60 | LCDR3 | QQNNEEPYT |

Refmab #1-Bert

| | | |
|---|---|---|
| 15 | VH | QVQLQQPGAELVKPGASVKMSCKASGYTFTSYNMHWVKQTP |
| | | GQGLEWIGVIYSGNGDTSYNQKFKGKATLTADKSSSTAYMQINS |
| | | LTSEDSAVYYCARERDTRFGNWGQGTLVTVSA |
| 63 | VL | EIVLTQSPSSLSASVGDRVTITCRASESVDIYGESFMHWYQQKPG |
| | | KAPKLLIYLASNLESGVPSRFSGSGSGTDFTLTISSLQPEDFATYYC |
| | | QQNKEEPYTFGQGTKLEIKRTV |
| 17 | HCDR1 | SYNMH |
| 18 | HCDR2 | VIYSGNGDTSYNQKFKG |
| 19 | HCDR3 | ERDTRFGN |
| 59 | LCDR1 | RASESVDIYGESFMH |
| 21 | LCDR2 | LASNLES |
| 61 | LCDR3 | QQNKEEPYT |

Refmab #1, Refmab #1-Ernie and Refmab #1-Bert were compared in various assay. Both derivatives have an IC50 in a SCF-dependent proliferation assay below that of the original Refmab #1 antibody on TF-1 cells (0.0176 and 0.0441 μg/ml compared to 0.2760 μg/ml) and on HSPC's (0.00521 and 0.0313 μg/ml compared to 0.0623 μg/ml). Both derivatives also inhibited SCF-dependent phosphorylation at least as strongly as the original Refmab #1 antibody. Both derivatives also inhibited binding of SCF to CD117. All antibodies also inhibit binding of SCF to CD117 in hematopoietic stem cells. All antibodies also deplete human HSC's in mice engrafted with human CD34+ HSPCs.

### Example 16: Refmab #1 Titration on Engineered TF-1 Cells

Binding of Refmab #1 was assessed in a flow cytometry assay. TF-1 cells were seeded at a cell concentration of 1,000,000 cells/ml in FACS Buffer (PBS+2% FCS+1 mM EDTA). After a Fc blocking using 1:20 diluted TruStain (BioLegend, #422302) in FACS buffer for 15 minutes at 4° C., cells were washed twice with FACS buffer. Subsequently, a SR-1 titration was added to the cells using 5-0.00015 μg/mL antibody for 30 minutes at 4° C. on a plate shaker. Cells were washed twice with FACS buffer and a secondary labelled antibody (IgG (H+L) Cross-Adsorbed Goat anti-Human, Alexa Fluor® 488, Invitrogen, A11013) was added to the cells for 30 minutes at 4° C. on a plate shaker. Cells were washed twice with FACS buffer, before adding 1:100 diluted solution of 7AAD staining (cell viability) in FACS buffer for a couple of minutes and analyzing the cells using the Novocyte Quanteon (Agilent).

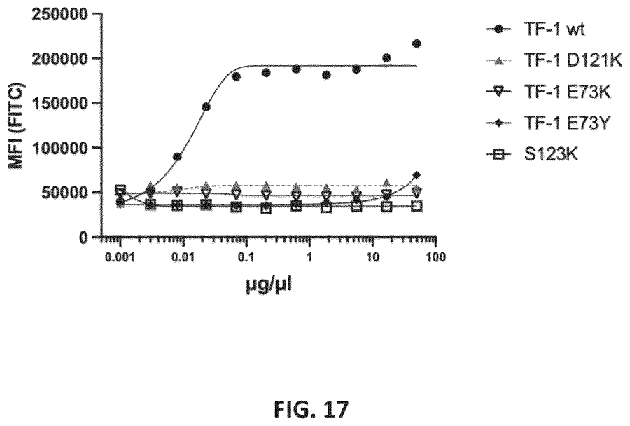

Results are shown in FIG. 17. Binding of the antibody Refmab #1 is completely abolished in TF-1 cells expressing CD117 variant E73K. Shown are results from two independent TF-1 E73K clones.

### Example 17: SCF-Dependent Phosphorylation of CD117

TF-1 cells (wild type, knock out, and variants E73K, D121K and S123K of CD117) were seeded at a concentration of 1 million cells/ml in 2 ml of Medium (RPMI1640 supplemented with GlutaMAX+10% heat inactivated FBS) in a 6-well plate. Cells were treated with 1-20 ng/ml recombinant human SCF (1 mg/ml PeproGMP Recombinant Human SCF) for 5 minutes, before cell collection. Cells were washed with ice-cold PBS, before resuspending cells in 500 μL Lysis Buffer (Lysis buffer from Signaling containing Protease Inhibitor and Phosphatase Inhibitor). Cells were flash frozen in liquid nitrogen and thawed at 37° C. twice, before freezing them at −80° C. until further usage. Undiluted samples were thawed and processed according to the manufacturers protocol for the PathScan Phospho-c-Kit (Tyr719) Sandwich ELISA Kit (Cell Signaling). Absorbance (OD_450 nm) was assessed using the Envision (Perkin Elmer).

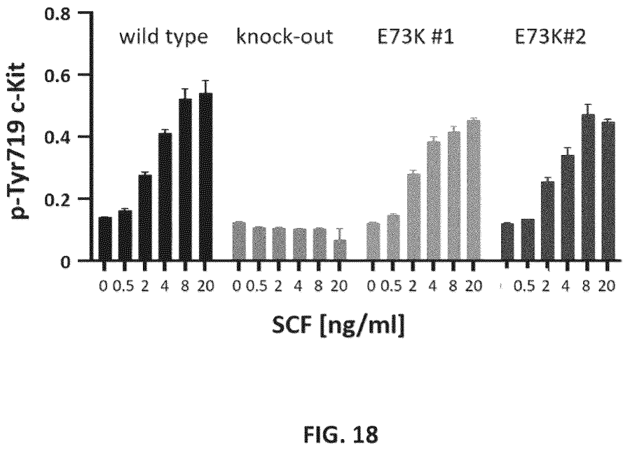

Exemplary results for variant E73K are shown on FIG. 18. Wild-type TF-1 cells and the E73K, D121K and S123K variants of CD117 showed a SCF-dependent phosphorylation at position Tyr719 of CD117. Phosphorylation levels of the knock-out cells are at the background level.

### Example 18: Preserved Signal Transduction of CD117 Variants in the Presence of Blocking Antibodies

TF-1 cells (wild type, and variants E73K, E73Y, D121K, S123F and S123K of CD117) were seeded at a concentration of 1 million cells/mL in 2 ml of Medium (RPMI1640 supplemented with GlutaMAX+10% heat inactivated FBS) in a 6-well plate. Cells were pre-treated with 0.5 μg/ml of antibodies Refmab #1-Ernie or Refmab #1-Bert for 30 minutes, before adding 100 ng/ml recombinant human SCF (1 mg/mL PeproGMP® Recombinant Human SCF) for 5 minutes. Cells were collected, washed once with ice-cold PBS and resuspended in 500 μL Lysis Buffer (Lysis buffer from Signaling containing Protease Inhibitor and Phosphatase Inhibitor). Cells were then flash frozen in liquid nitrogen and thawed at 37° C. twice, before freezing them at −80° C. until further usage. Undiluted samples were thawed and processed according to the manufacturers protocol for the PathScan Phospho-c-Kit (Tyr719) Sandwich ELISA Kit (Cell Signaling) or the Human c-Kit (CD117) ELISA Kit (Abcam). Absorbance (OD_450 nm) was assessed using the Envision (Perkin Elmer).

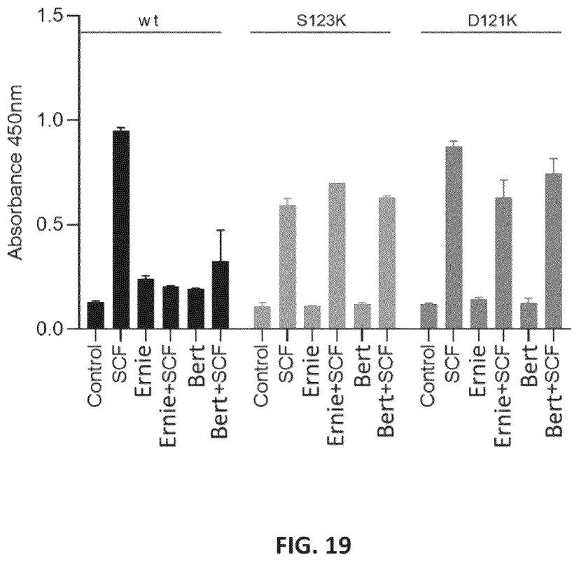

Exemplary results for variants D121K and S123K are shown in FIG. 19. SCF-dependent CD117 phosphorylation in wild-type TF-1 cells is blocked by antibodies Refmab #1-Ernie and Refmab #1-Bert, whereas in TF-1 cells with D121K and S123K variants of CD117 phosphorylation is unaffected. The same is also observed for variants E73K, E73Y and S123F (data not shown).

### Example 19: HSC Depletion Experiment

Depletion of human HSCs in mice using various mAbs was adapted from Pang et al. (Blood (2019) 133:2069-78).

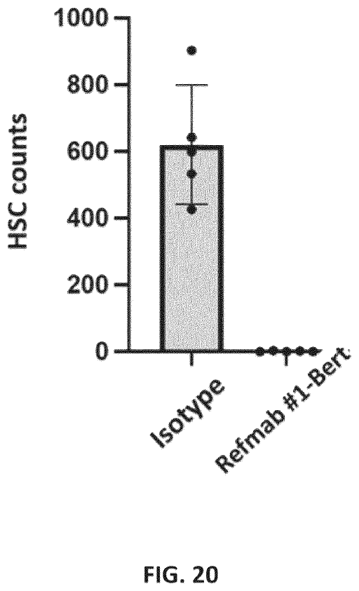

NBSGW mice (Jackson Laboratories) were injected with 1 Mio HSPCs. 8, 10, 12 and 14 days after cell injection mice received 25 mg/kg antibody Refmab #1-Bert per dose i.v. 16 weeks later the mice were euthanized and blood, spleen and bone marrow were analysed by FACS. Results for bone marrow HSC depletion are shown in FIG. 20. The figure shows in vivo depletion of HSCs after injection of Refmab #1-Bert, compared to animals receiving an isotype control antibody. HSCs were identified by FACS as live/hCD45+/CD34+/CD38−/CD45RA−/CD90+.

In another experiment, NSG mice (Jackson Laboratories) were sub-lethally irradiated one day before injection with 1 Mio gene-edited HSPCs carrying a E73K variant of CD117. 8, 10, 12 and 14 days after cell injection mice received 25 mg/kg antibody Refmab #1-Bert or 25 mg/kg isotope control antibody i.v. per dose. 16 weeks later the mice were euthanized, and blood, spleen and bone marrow were analyzed by FACS.

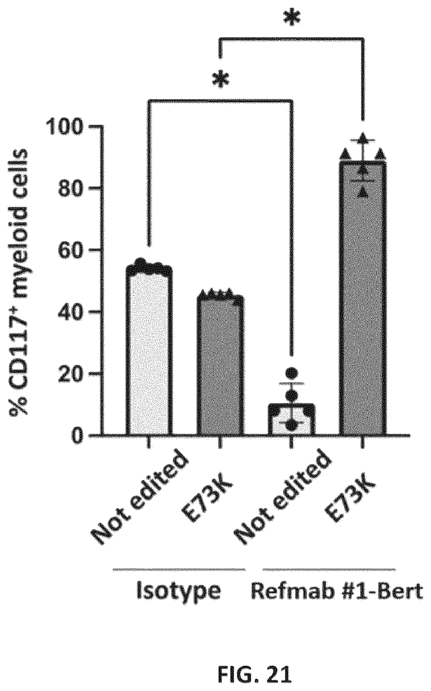

Results for bone marrow are shown in FIG. 21. CD117+ myeloid cells were identified by FACS as live/hCD45+/CD33+/CD117+. To identify CD117+ cells the antibody clone 104D2 was used, which does not interfere with binding of Refmab #1 and Refmab #1-Bert to CD117. Clone 104D2 was used as an expression control. Cells were classified as not edited (104D2+ and SR-1+) or E73K edited (104D2+ but SR-1−) based on double staining with anti-CD117 clone SR-1 and anti-CD117 clone 104D2.

FIG. 21 shows depletion of unedited cells in mice receiving antibody Refmab #1-Bert compared to mice receiving an isotype control antibody. In contrast, injection of Refmab #1-Bert resulted in an enrichment of E73K variant cells compared to animals receiving isotype control antibody.

### Example 20: CD117 Variants are Resistant to Treatment with ADCs

In this experiment, chicken DF-1 cells were used. DF-1 cells lack CD117. Cells were transfected with selected CD117 variants (E73Y, D121K and S123K). Transfected cells were treated with ADCs (antibodies Refmab #1-Ernie and Refmab #1-Bert, both coupled to teserine) at a concentration of 0.1, 1 and 10 μg/ml. After 48 hours cells were analyzed by FACS.

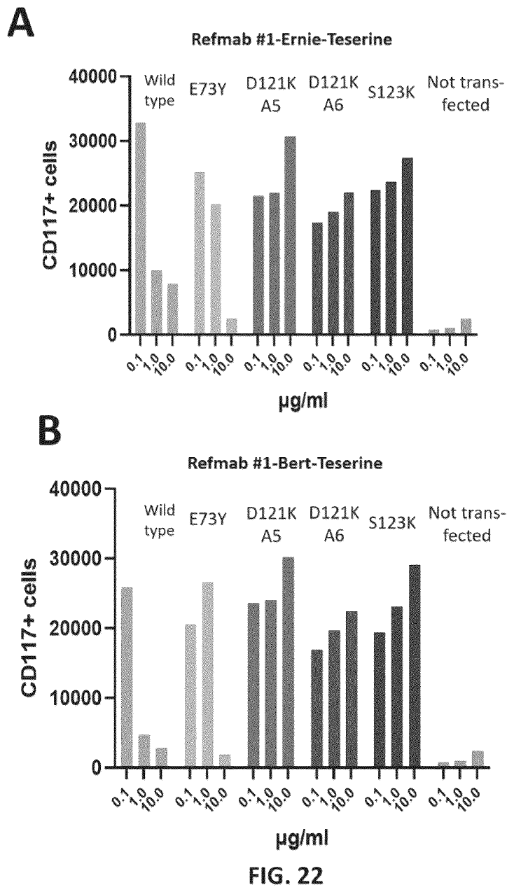

Results are shown in FIG. 22. D121K A5 and D121K A6 designate two different batches of plasmids for the same variant. Both tested ADCs, Refmab #1-Ernie-teserine and Refmab #1-Bert-teserine, led to an effective depletion of wild-type DF-1 cells, but not DF-1 cells transfected with D121K or S123K variants of CD117.

### Example 21: Additional HSC Depletion Experiment

Depletion of human HSCs in mice using various mAbs was adapted from Pang et al. (Blood (2019) 133:2069-78).

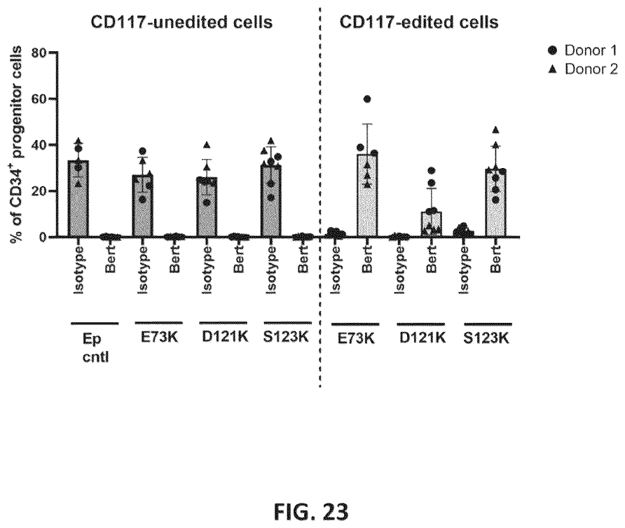

NBSGW mice (Jackson Laboratories) were injected with 1 Mio HSPCs from 2 different donors carrying a E73K, S123K or D121K variant of CD117. Control groups of mice received unedited electroporation control HSPCs. 7, 9, 11 and 12 days after cell injection mice received 4 mg/kg antibody Refmab #1-Bert per dose i.v. 16 weeks later the mice were euthanized and blood, spleen and bone marrow were analyzed by FACS. Results for depletion of CD117-unedited cells and enrichment of CD117-edited cells are shown in FIG. 23. To identify unedited and edited cells the CD117-specific antibody clones 104D2 and SR-1 were used. Binding of 104D2 is not affected by the genetic editing, whereas SR-1 does not bind to the edited variants of CD117. Cells were classified as unedited (104D2+ and SR-1+) or edited (104D2+ but SR-1−) based on double staining with both anti-CD117 clones.

FIG. 23 shows depletion of unedited CD34+ progenitor cells (Gated as live/hCD45+/CD34+/CD38−) in mice receiving antibody Refmab #1-Bert compared to mice receiving an isotype control antibody. In contrast, injection of Refmab #1-Bert resulted in an enrichment of E73K, D121K and S123K-edited variant cells compared to animals receiving isotype control antibody.

1. A mammalian cell or a population of cells expressing a first isoform of CD117 for use in a medical treatment in a patient in need thereof, said patient having cells expressing a second isoform of CD117,
wherein said cell expressing said first isoform comprises genomic DNA with at least one polymorphism or genetically engineered allele,
wherein said polymorphism or genetically engineered allele is not present in the genome of the patient having cells expressing said second isoform of CD117,
wherein said polymorphic or genetically engineered allele is characterized by at least one substitution of an amino acid in position E73, D121, R122, S123, S239, Y259 and/or S261 of SEQ ID NO: 1.

2. The mammalian cell or population of cells for use according to claim 1,
wherein said medical treatment comprises administering a therapeutically efficient amount of said cell or population of cells expressing said first isoform of CD117 to said patient in need thereof, in combination with a therapeutically efficient amount of a depleting agent comprising an antigen-binding region that binds specifically to said second isoform of CD117 to specifically deplete patient cells expressing said second isoform of CD117.

3. The mammalian cell or population of cells for use according to claim 1, wherein said first and second isoforms are substantially functionally identical.

4. The mammalian cell or population of cells for use according to claim 1, wherein
said first and second isoforms bind to SCF, lead to SCF-dependent proliferation and/or lead to SCF-dependent phosphorylation.

5. The mammalian cell or population of cells for use according to claim 1, wherein said first and said second isoform of CD117 bind to SCF at a substantially similar degree, preferably wherein the KD of said first isoform of CD117 to SCF is less than four-fold, preferably less than three-fold, more preferably less than two-fold and most preferably less than 1.5-fold higher, than the KD of the second isoform of CD117 to SCF.

6. The mammalian cell or population of cells for use according to claim 1, wherein residue E73 is substituted with an amino acid selected from the group consisting of K, L, Y, and R, and/or residue D121 is substituted with an amino acid selected from the group consisting of Y, H, K, R or T, and most preferably H or K, and/or said residue S123 is substituted with an amino acid selected from the group consisting of P, F or K, preferably K, and/or said residue S239 is substituted with H or K and/or said residue Y259 is substituted with an amino acid selected from the group consisting of E, A, G, P, C and H, preferably P, A or G, and most preferably A, and/or said residue K193 is substituted with an amino acid selected from the group consisting G, T, M, D and E.

7. The mammalian cell or population of cells for use according to claim 1, wherein said cell expressing said first isoform of CD117 has been selected from a subject comprising native genomic DNA with at least one natural polymorphism allele in nucleic acid encoding said first isoform.

8. The mammalian cell or population of cells for use according to claim 1, wherein said first isoform of CD117 is obtained by modifying the nucleic acid sequence encoding said first isoform of CD117 by gene editing, preferably by introducing into a cell a gene editing enzyme capable of inducing site-specific mutations(s) within a target sequence encoding a CD117 surface protein region involved in the binding of agent comprising at least a first antigen-binding region.

9. The mammalian cell or population of cells according to claim 1, wherein said medical treatment restores normal haematopoiesis after immunotherapy in the treatment of hematopoietic disease, and preferably in the treatment of malignant hematopoietic disease such as acute myeloid leukemia (AML), myelodysblastic syndrome (MDS), systemic mastocytosis, chronic myeloid leukemia (CML), blastic plasmacytoid dendritic cell neoplasm (BPDCN), or B-acute lymphoblastic leukemia (B-ALL).

10. The mammalian cell or population of cells for use according to claim 2, wherein said depleting agent is an antibody, antibody-drug conjugate or an immune cell, preferably a T-cell bearing a chimeric antigen receptor (CAR) comprising a first antigen-binding region which binds specifically to said second isoform and does not bind or binds substantially weaker to said first isoform.

11. The mammalian cell or population of cells for use according to claim 2, wherein said first antigen-binding region of said depleting agent binds specifically to an epitope including the amino acids E73, D121, R122, S123, S239, Y259 and/or S261 of SEQ ID NO: 1, more preferably to an epitope including the amino acids E73, S123, K127 and/or Y259 of SEQ ID NO: 1.

12. The mammalian cell or population of cells for use according to claim 2, wherein said first antigen-binding region comprises:
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
an antibody light chain variable domain (VL) comprising the three CDRs VLCDR1, VLCDR2 and VLCDR3 wherein VLCDR1 is SEQ ID NO: 20, VLCDR2 is SEQ ID NO: 21, VLCDR3 is SEQ ID NO: 22, or
b) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60, or
a) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61.

13. The mammalian cell or population of cells for use according to claim 12, wherein said first antigen-binding region comprises
a) a heavy chain variable domain comprising or consisting of an amino acid sequence of SEQ ID NO: 15 and a light chain variable domain comprising or consisting of an amino acid sequence of SEQ ID NO: 16, or
b) a heavy chain variable domain comprising or consisting of an amino acid sequence of SEQ ID NO: 15 and a light chain variable domain comprising or consisting of an amino acid sequence of SEQ ID NO: 62, or
c) a heavy chain variable domain comprising or consisting of an amino acid sequence of SEQ ID NO: 15 and a light chain variable domain comprising or consisting of an amino acid sequence of SEQ ID NO: 63.

14. The mammalian cell or population of cells for use according to claim 1, wherein said medical treatment comprises:
administering a therapeutically efficient amount of said cell or population of cells expressing said first isoform of CD117 to said patient in need thereof in combination with a therapeutically efficient amount of a depleting agent comprising an antigen-binding region that binds specifically to said first isoform of CD117 to specifically deplete transferred cells expressing first isoform of CD117.

15. The mammalian cell or population of cells for use according to claim 14, wherein said use is the use in adoptive cell transfer therapy, preferably for the treatment of malignant hematopoietic disease such as acute myeloid leukemia (AML), blastic plasmacytoid dendritic cell neoplasm (BPDCN), or B-acute lymphoblastic leukemia (B-ALL).

16. The mammalian cell or population of cells for use according to claim 14, wherein said depleting agent is administered subsequently to said cell or population of cells expressing said first isoform of CD117 to avoid eventual severe side effects such as graft-versus-host disease due to the transplantation.

17. The mammalian cell or population of cells for use according to claim 14, wherein said cell or population of cells expressing said first isoform is an immune cell, preferably a T-cell, bearing a chimeric antigen receptor (CAR).

18. A pharmaceutical composition comprising a mammalian cell or population of cells according to claim 1, optionally a depleting agent and a pharmaceutically acceptable carrier.

19. A depleting agent for use in preventing or reducing the risk of severe side effects in a patient having received a cell expressing a first isoform of CD117, wherein said patient's native cells express a second isoform of CD117, and wherein said depleting agent comprises at least a second antigen-binding region which binds specifically to said first isoform of CD117 and does not bind or binds substantially weaker to said second isoform of CD117.

20. A depleting agent for use in selectively depleting the host cells in a patient in need thereof wherein said patient's native cells express a second isoform of CD117 and wherein said depleting agent comprises at least a antigen-binding region which binds specifically to said second isoform of CD117, and wherein said first antigen-binding region of said depleting binds specifically to an epitope including the amino acids E73, D121, R122, S123, S239, Y259 and/or S261 of SEQ ID NO: 1, more preferably to an epitope including the amino acids E73, S123, K127 and/or Y259 of SEQ ID NO: 1.

21. A depleting agent for use according to claim 20, wherein said antigen-binding region comprises:
a) an antibody heavy chain variable domain (VH) comprising the three CDRs VHCDR1, VHCDR2 and VHCDR3 wherein VHCD1 is SEQ ID NO: 17, VHCD2 is SEQ ID NO: 18 and VHCDR3 is SEQ ID NO: 19; and
an antibody light chain variable domain (VL) comprising the three CDRs VLCDR1, VLCDR2 and VLCDR3 wherein VLCDR1 is SEQ ID NO: 20, VLCDR2 is SEQ ID NO: 21, VLCDR3 is SEQ ID NO: 22, or
b) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60, or
c) an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19; and
an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61.

22. A depleting agent for use according to claim 21, wherein said antigen-binding region comprises:
a) a heavy chain variable domain comprising or consisting of an amino acid sequence of SEQ ID NO: 15 and a light chain variable domain comprising or consisting of an amino acid sequence of SEQ ID NO: 16, or
b) a heavy chain variable domain comprising or consisting of an amino acid sequence of SEQ ID NO: 15 and a light chain variable domain comprising or consisting of an amino acid sequence of SEQ ID NO: 62, or
c) a heavy chain variable domain comprising or consisting of an amino acid sequence of SEQ ID NO: 15 and a light chain variable domain comprising or consisting of an amino acid sequence of SEQ ID NO: 63.

23. A combination of
a) a mammalian cell or a population of cells expressing an isoform of CD117, wherein said isoform of CD117 is characterized by a substitution of the aspartic acid at position 121 of wild type CD117 to a lysine, and
b) a depleting agent comprising
i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22,
ii. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60, or
iii. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61.
for use in a medical treatment in a patient in need thereof.

24. A combination of
a) a mammalian cell or a population of cells expressing an isoform of CD117, wherein said isoform of CD117 is characterized by a substitution of the serine at position 123 of wild type CD117 to a lysine, and
b) a depleting agent comprising
i. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
an antibody light chain variable domain (VL) comprising the three CDRs VLCDR1, VLCDR2 and VLCDR3 wherein VLCDR1 is SEQ ID NO: 20, VLCDR2 is SEQ ID NO: 21 and VLCDR3 is SEQ ID NO: 22,
ii. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 60, or
iii. an antibody heavy chain variable domain (VH) comprising the three CDRs VHCDR1, VHCDR2 and VHCDR3 wherein VHCDR1 is SEQ ID NO: 17, VHCDR2 is SEQ ID NO: 18 and VHCDR3 is SEQ ID NO: 19, and
an antibody light chain variable domain (VL) comprising the three CDRs VLCDR1, VLCDR2 and VLCDR3 wherein VLCDR1 is SEQ ID NO: 59, VLCDR2 is SEQ ID NO: 21 and VLCDR3 is SEQ ID NO: 61.
for use in a medical treatment in a patient in need thereof.


SEQUENCE LISTING (67 sequences)

SEQ ID NO: 1 - protein, 976 aa, Homo sapiens [sequence omitted]

SEQ ID NO: 2 - protein, 273 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFVVASETSDCVVSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 3 - DNA, 20 bp, synthetic construct
  misc_feature 1..20: note = KIT_E73_1
  source 1..20: mol_type = other DNA
ctgatccgggctttgtcaaa

SEQ ID NO: 4 - DNA, 20 bp, synthetic construct
  misc_feature 1..20: note = KIT_E73_2
  source 1..20: mol_type = other DNA
aaagtccatttgacaaagcc

SEQ ID NO: 5 - DNA, 20 bp, synthetic construct
  misc_feature 1..20: note = KIT_E73_3
  source 1..20: mol_type = other DNA
caaatggacttttgagatcc

SEQ ID NO: 6 - DNA, 20 bp, synthetic construct
  misc_feature 1..20: note = KIT_E73_4
  source 1..20: mol_type = other DNA
tattctcattcgtttcatcc

SEQ ID NO: 7 - DNA, 20 bp, synthetic construct
  misc_feature 1..20: note = KIT_E73_5
  source 1..20: mol_type = other DNA
atgagaataagcagaatgaa

SEQ ID NO: 8 - DNA, 20 bp, synthetic construct
  misc_feature 1..20: note = KIT_E73_6
  source 1..20: mol_type = other DNA
taagcagaatgaatggatca

SEQ ID NO: 9 - DNA, 20 bp, synthetic construct
  misc_feature 1..20: note = KIT_120_123_1
  source 1..20: mol_type = other DNA
gttgtcttctttcccataca

SEQ ID NO: 10 - DNA, 20 bp, synthetic construct
  misc_feature 1..20: note = KIT_120_123_2
  source 1..20: mol_type = other DNA
cttctttcccatacaaggag

SEQ ID NO: 11 - DNA, 20 bp, synthetic construct
  misc_feature 1..20: note = KIT_120_123_3
  source 1..20: mol_type = other DNA
ttgttgaccgctccttgtat

SEQ ID NO: 12 - DNA, 20 bp, synthetic construct
  misc_feature 1..20: note = KIT_120_123_4
  source 1..20: mol_type = other DNA
cttgttgaccgctccttgta

SEQ ID NO: 13 - DNA, 20 bp, synthetic construct
  misc_feature 1..20: note = KIT_120_123_5
  source 1..20: mol_type = other DNA
catacaaggagcggtcaaca

SEQ ID NO: 14 - DNA, 20 bp, synthetic construct
  misc_feature 1..20: note = KIT_120_123_6
  source 1..20: mol_type = other DNA
gcggtcaacaaggaaaagct

SEQ ID NO: 15 - protein, 117 aa, synthetic construct
  REGION 1..117: note = Refmab #1 VH
QVQLQQPGAELVKPGASVKMSCKASGYTFTSYNMHWVKQTPGQGLEWIGVIYSGNGDTSYNQKFKGKATLTADKSSSTAYMQINSLTSEDSAVYYCARERDTRFGNWGQGTLVTVSA

SEQ ID NO: 16 - protein, 111 aa, synthetic construct
  REGION 1..111: note = Refmab #1 VL
NIVLTQSPASLAVSLGLRATISCRASESVDIYGNSFMHWYQQKPGQPPKLLIYLASNLESGVPARFSGSGSRTDFTLTIDPVEADDAATYYCQQNNEDPYTFGGGTKLEIK

SEQ ID NO: 17 - protein, 5 aa, synthetic construct
  REGION 1..5: note = Refmab #1 HCDR1
SYNMH

SEQ ID NO: 18 - protein, 17 aa, synthetic construct
  REGION 1..17: note = Refmab #1 HCDR2
VIYSGNGDTSYNQKFKG

SEQ ID NO: 19 - protein, 8 aa, synthetic construct
  REGION 1..8: note = Refmab #1 HCDR3
ERDTRFGN

SEQ ID NO: 20 - protein, 15 aa, synthetic construct
  REGION 1..15: note = Refmab #1 LCDR1
RASESVDIYGNSFMH

SEQ ID NO: 21 - protein, 7 aa, synthetic construct
  REGION 1..7: note = Refmab #1 LCDR2
LASNLES

SEQ ID NO: 22 - protein, 9 aa, synthetic construct
  REGION 1..9: note = Refmab #1 LCDR3
QQNNEDPYT

SEQ ID NO: 23 - protein, 118 aa, synthetic construct
  REGION 1..118: note = Refmab #2 VH
QVQLVQSGAEVKKPGSSVKVSCKASGGTFSSYAISWVRQAPGQGLEWMGTIGPFEGQPRYAQKFQGRVTITADESTSTAYMELSSLRSEDTAVYYCARGGYISDFDVWGQGTLVTVSS

SEQ ID NO: 24 - protein, 108 aa, synthetic construct
  REGION 1..108: note = Refmab #2 VL
DIQMTQSPSSLSASVGDRVTITCRASQSISNYLAWYQQKPGKAPKLLIYDASSLQSGVPSRFSGSGSGTDFTLTISSLQPEDFATYYCQQYYYESITFGQGTKVEIKR

SEQ ID NO: 25 - protein, 5 aa, synthetic construct
  REGION 1..5: note = Refmab #2 HCDR1
SYAIS

SEQ ID NO: 26 - protein, 17 aa, synthetic construct
  REGION 1..17: note = Refmab #2 HCDR2
TIGPFEGQPRYAQKFQG

SEQ ID NO: 27 - protein, 9 aa, synthetic construct
  REGION 1..9: note = Refmab #2 HCDR3
GGYISDFDV

SEQ ID NO: 28 - protein, 11 aa, synthetic construct
  REGION 1..11: note = Refmab #2 LCDR1
RASQSISNYLA

SEQ ID NO: 29 - protein, 7 aa, synthetic construct
  REGION 1..7: note = Refmab #2 LCDR2
DASSLQS

SEQ ID NO: 30 - protein, 9 aa, synthetic construct
  REGION 1..9: note = Refmab #2 LCDR3
QQYYYESIT

SEQ ID NO: 31 - protein, 123 aa, synthetic construct
  REGION 1..123: note = Refmab #3 VH
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 32 - protein, 107 aa, synthetic construct
  REGION 1..107: note = Refmab #3 VL
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 33 - protein, 5 aa, synthetic construct
  REGION 1..5: note = Refmab #3 HCDR1
NYWIG

SEQ ID NO: 34 - protein, 17 aa, synthetic construct
  REGION 1..17: note = Refmab #3 HCDR2
IINPRDSDTRYRPSFQG

SEQ ID NO: 35 - protein, 14 aa, synthetic construct
  REGION 1..14: note = Refmab #3 HCDR3
HGRGYEGYEGAFDI

SEQ ID NO: 36 - protein, 11 aa, synthetic construct
  REGION 1..11: note = Refmab #3 LCDR1
RSSQGIRSDLG

SEQ ID NO: 37 - protein, 7 aa, synthetic construct
  REGION 1..7: note = Refmab #3 LCDR2
DASNLET

SEQ ID NO: 38 - protein, 9 aa, synthetic construct
  REGION 1..9: note = Refmab #3 LCDR3
QQANGFPLT

SEQ ID NO: 39 - protein, 117 aa, synthetic construct
  REGION 1..117: note = Refmab #4 VH
EVQLVESGGGLVQPGGSLRLSCAASGFSISVYMMHWVRQAPGKGLEWVASIYPYSGYTYYADSVKGRFTISADTSKNTAYLQMNSLRAEDTAVYYCARYVYHALDYWGQGTLVTVSS

SEQ ID NO: 40 - protein, 109 aa, synthetic construct
  REGION 1..109: note = Refmab #4 VL
DIQMTQSPSSLSASVGDRVTITCRASQSVSSAVAWYQQKPGKAPKLLIYSASSLYSGVPSRFSGSRSGTDFTLTISSLQPEDFATYYCQQWAVHSLITFGQGTKVEIKR

SEQ ID NO: 41 - protein, 5 aa, synthetic construct
  REGION 1..5: note = Refmab #4 HCDR1
VYMMH

SEQ ID NO: 42 - protein, 17 aa, synthetic construct
  REGION 1..17: note = Refmab #4 HCDR2
SIYPYSGYTYYADSVKG

SEQ ID NO: 43 - protein, 8 aa, synthetic construct
  REGION 1..8: note = Refmab #4 HCDR3
YVYHALDY

SEQ ID NO: 44 - protein, 11 aa, synthetic construct
  REGION 1..11: note = Refmab #4 LCDR1
RASQSVSSAVA

SEQ ID NO: 45 - protein, 7 aa, synthetic construct
  REGION 1..7: note = Refmab #4 LCDR2
SASSLYS

SEQ ID NO: 46 - protein, 10 aa, synthetic construct
  REGION 1..10: note = Refmab #4 LCDR3
QQWAVHSLIT

SEQ ID NO: 47 - protein, 122 aa, synthetic construct
  REGION 1..122: note = Refmab #5 VH
EVRLVESEGGLVQPRSSMKLSCTASGFTFSDYYMAWVRQVPEKGLEWVANINYDGSSTYYLDSLKSRFIISRDNAKNILYLQMSSLKSEDTATYYCARGDYYGTTYWYFDVWGTGTTVTVSS

SEQ ID NO: 48 - protein, 107 aa, synthetic construct
  REGION 1..107: note = Refmab #5 VL
DIQMTQTTSSLSASLGDRVTISCRASQDISNYLNWYQQKPDGTVKLLIYYTSRLQSGVPSRFSGSGSGTDYSLTISNLEQEDIATYFCQQGKKLWSFGGGTKLEIKR

SEQ ID NO: 49 - protein, 5 aa, synthetic construct
  REGION 1..5: note = Refmab #5 HCDR1
DYYMA

SEQ ID NO: 50 - protein, 17 aa, synthetic construct
  REGION 1..17: note = Refmab #5 HCDR2
NINYDGSSTYYLDSLKS

SEQ ID NO: 51 - protein, 13 aa, synthetic construct
  REGION 1..13: note = Refmab #5 HCDR3
GDYYGTTYWYFDV

SEQ ID NO: 52 - protein, 11 aa, synthetic construct
  REGION 1..11: note = Refmab #5 LCDR1
RASQDISNYLN

SEQ ID NO: 53 - protein, 7 aa, synthetic construct
  REGION 1..7: note = Refmab #5 LCDR2
YTSRLQS

SEQ ID NO: 54 - protein, 8 aa, synthetic construct
  REGION 1..8: note = Refmab #5 LCDR3
QQGKKLWS

SEQ ID NO: 55 - DNA, 84 bp, synthetic construct
  misc_feature 1..84: note = E73K-CIT03_2-plus
  source 1..84: mol_type = other DNA
ctgatccgggctttgtcaaatggacttttgagatattggataaaacgaatgagaataagcagaatgaatggatcacggaaaagg

SEQ ID NO: 56 - DNA, 84 bp, synthetic construct
  misc_feature 1..84: note = E73K-CIT03_2-min
  source 1..84: mol_type = other DNA
ccttttccgtgatccattcattctgcttattctcattcgttttatccaatatctcaaaagtccatttgacaaagcccggatcag

SEQ ID NO: 57 - DNA, 140 bp, synthetic construct
  misc_feature 1..140: note = E73K-CIT03_2-plus_140bp
  source 1..140: mol_type = other DNA
gtgggcgacgagattaggctgttatgcactgatccgggctttgtcaaatggacttttgagatattggataaaacgaatgagaataagcagaatgaatggatcacggaaaaggcagaagccaccaacaccggcaaatacac

SEQ ID NO: 58 - DNA, 140 bp, synthetic construct
  misc_feature 1..140: note = E73K-CIT03_2-min_140bp
  source 1..140: mol_type = other DNA
gtgtatttgccggtgttggtggcttctgccttttccgtgatccattcattctgcttattctcattcgttttatccaatatctcaaaagtccatttgacaaagcccggatcagtgcataacagcctaatctcgtcgcccac

SEQ ID NO: 59 - protein, 15 aa, synthetic construct
RASESVDIYGESFMH

SEQ ID NO: 60 - protein, 9 aa, synthetic construct
QQNNEEPYT

SEQ ID NO: 61 - protein, 9 aa, synthetic construct
QQNKEEPYT

SEQ ID NO: 62 - protein, 114 aa, synthetic construct
EIVLTQSPSSLSASVGDRVTITCRASESVDIYGESFMHWYQQKPGKAPKLLIYLASNLESGVPSRFSGSGSGTDFTLTISSLQPEDFATYYCQQNNEEPYTFGQGTKLEIKRTV

SEQ ID NO: 63 - protein, 114 aa, synthetic construct
EIVLTQSPSSLSASVGDRVTITCRASESVDIYGESFMHWYQQKPGKAPKLLIYLASNLESGVPSRFSGSGSGTDFTLTISSLQPEDFATYYCQQNKEEPYTFGQGTKLEIKRTV

SEQ ID NO: 64 - DNA, 117 bp, synthetic construct
  source 1..117: mol_type = other DNA
gacgagattaggctgttatgcactgatccgggctttgtcaaatggacttttgagatcctggatgaaacgaatgagaataagcagaatgaatggatcacggaaaaggcagaagccacc

SEQ ID NO: 65 - protein, 117 aa, synthetic construct
ASPGLUILEARGLEULEUCYSTHRASPPROGLYPHEVALLYSTRPTHRPHEGLUILELEUASPGLUTHRASNGLUASNLYSGLNASNGLUTRPILETHRGLULYSALAGLUALATHR

SEQ ID NO: 66 - DNA, 94 bp, synthetic construct
  source 1..94: mol_type = other DNA
cctgccaagccttttccttgttgaccgctccttgtatgggaaagaagacaacgacacgctggtccgctgtcctctcacagacccagaagtgacc

SEQ ID NO: 67 - protein, 93 aa, synthetic construct
PROALALYSLEUPHELEUVALASPARGSERLEUTYRGLYLYSGLUASPASNASPTHRLEUVALARGCYSPROLEUTHRASPPROGLUVALTHR